(19) United States
(12) Patent Application Publication
(10) Pub. No.: US 2003/0190739 A1
(43) Pub. Date: Oct. 9, 2003

(54) Tankyrase2 materials and methods

(76) Inventors: Erik Christenson, Bellevue, WA (US); Anthony J. Demaggio, Kirkland, WA (US); Phyllis S. Goldman, Bothell, WA (US); David L. McElligott, Bothell, WA (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/199,937

(22) Filed: Jul. 22, 2002

Related U.S. Application Data
(63) Continuation of application No. 09/606,035, filed on Jun. 28, 2000, now abandoned.
(60) Provisional application No. 60/141,582, filed on Jun. 29, 1999.

(51) Int. Cl. C12Q 1/68; C07H 21/04; C12N 9/64; C12P 21/02; C12N 5/06

(52) U.S. Cl. 435/226; 435/69.1; 435/6; 435/320.1; 435/325; 536/23.2

(57) ABSTRACT

The invention provides novel tankyrase polypeptides designated tankyrase2, polynucleotides encoding the polypeptides, expression constructs comprising the polynucleotides, and host cells transformed with the expression constructs. Also provided are methods for producing the tankyrase2 polypeptides, antibodies that are immunoreactive with the tankyrase2 polypeptides. In addition, there are provided methods for identifying specific binding partners of tankyrase2, and more particularly methods for identifying binding partners that modulate biological activity of tankyrase2. Methods of modulating biological activity of tankyrase2 in vitro and in vivo are also provided.

This application claims the benefit of U.S. Provisional Application Serial No. 60/141,582, filed Jun. 29, 1999.

The present invention relates generally to a novel tankyrase polypeptide having poly ADP-ribosylation activity, to polynucleotides encoding the polypeptide, and to methods of using such materials.

### BACKGROUND OF THE INVENTION

The ends of eukaryotic chromosomes (telomeres) are characterized by simple repeat DNA sequences. The length and sequence of the repeats varies from species to species but the importance of telomeres is universal in organisms with linear chromosomes. Telomeres protect the ends of the chromosomes and ostensibly function to prevent recombination of chromosome ends, which leads to chromosomal fusion and instability. In addition, there is considerable evidence that the length of the telomere repeats determines the ability of a cell to divide or perhaps even to survive.

The telomeres of cultured primary human fibroblasts become progressively shorter with each cell division in the absence of an active mechanism to regenerate telomere length [Harley et al., Nature 345:458-60 (1990)]. At some critical stage of telomere shortening, these cells are no longer able to divide and enter a state known as cellular senescence. Thus, in human primary fibroblasts at least, telomere length functions as a biological clock to monitor cellular aging and regulate longevity.

The observation that telomere length regulates cellular aging prompted the hypothesis that telomere regulation may also be critical for organismal aging. Mice that are unable to replicate telomeres show characteristics of premature aging after the third generation. These characteristics include premature graying, decreased cell division capacity, impaired wound healing, and increased cancer incidence amongst others. Thus, regulation of telomere structure may be critical for some of the characteristics associated with aging. Drugs that modulate the regulation of telomere structure thus may have utility in treatment of age-related syndromes or in cases of genetically determined premature aging syndromes.

Only recently has some of the machinery that replicates telomeres been described. This machinery, collectively referred to as the telomeres complex, consists of several proteins that replicate the telomeres and protect the telomere structure from DNA repair, which otherwise might treat telomeres as damaged DNA and affect end joining or recombination thus destroying the integrity of the chromosome. Telomerase is the replicative component of the telomerase complex and is a DNA polymerase that features an integral RNA molecule that serves as the template for the addition of the repetitive sequences [for a review, see Greider, Ann Rev Biochem 65:337-65 (1996)]. The observation that telomerase activity is essential for continued cell division suggests that inappropriate telomerase activity may be, in some instances, a contributing factor in the oncogenic transformation of cells. Forced expression of telomerase does not in and of itself cause oncogenic transformation but the fact that cells overexpressing telomerase have apparently unlimited capacity to replicate suggests that inappropriate expression of telomerase may be one step in a multi-step process of oncogenic transformation. In addition, numerous studies have shown that telomerase activity is higher in tumor tissue than most normal tissues suggesting that increased telomerase activity may be essential for tumor growth [for reviews, see Bacchetti, Cancer Surv 28:197-216 (1996); and Harley et al., Cold Spring Harbor Symp Quant Biol 59:307-15(1994)].

Two telomere-specific DNA binding proteins, designated TRF1 and TRF2 have also been shown to be important for maintenance of telomeres [Chong et al., Science 270:1663-7 (1995); van Steensel et al., Cell 92:401-13 (1998)]. TRF1 has a critical role in the regulation of telomere length while TRF2 seems to be important for protecting chromosome ends. Both molecules contain DNA binding domains and dimerization domains and both appear to function as homodimers. Binding of TRF1 to telomere repeats inhibits the function of telomerase thus contributing to telomere shortening during replication [van Steensel and de Lange, Nature 385:740-3 (1997)].

An additional molecule, tankyrase, has been identified which modifies TRF1 by the addition of polymers of ADP-ribose [Smith et al., Science 282:1484-7 (1998)]. Tankyrase is structurally and functionally related to the Poly(ADP-Ribose) Polymerase (PARP) molecule, which modifies proteins by the addition of ADP-ribose polymers [for review see Alvarez-Gonzalez et al., Mol Cell Biochem 138:33-7 (1994)]. The structural relationship to PARP exists in a putative catalytic domain of tankyrase that has extensive amino acid sequence similarity to PARP. In addition, tankyrase contains a sterile alpha motif (SAM) and 24 ankyrin repeats. These structures are typically involved in protein/protein interactions and at least a portion of the ankyrin repeat region in tankyrase has been shown to be responsible for the interaction with TRF1. Tankyrase has been shown to poly ADP-ribosylate TRF1 in vitro and it has been suggested that the role of tankyrase in vivo is to ADP-ribosylate TRF1 causing dissociation of TRFL from the telomere repeats and thus allowing telomerase to replicate the telomeres. Drugs that inhibit tankyrase activity then might be expected to inhibit the replication of telomeres and thus cause eventual senescence of dividing cell populations such as cancer cells or proliferating immune system cell as examples.

As tankyrase or tankyrase-related gene products might be attractive targets of drug design, there is a need in the art to identify additional molecules with related functions and/or structures. Such molecules might serve as specificity controls for tankyrase targeted drugs or may themselves be suitable targets for drug discovery programs.

In view of the above considerations, it is clear that existing knowledge is lacking with respect to cellular DNA repair mechanisms, signaling, and induction of cellular replication, mechanisms of tumorigenesis, and treatment of cancer disease states. Thus, there exists a need in the art for the identification of additional tankyrase-like molecules for use in determining the selectivity of therapeutics designed to modulate tankyrase function and as targets in their own right for therapeutic intervention in human diseases. The profiling of tankyrase inhibitors on additional tankyrase gene products may allow for the tankyrase-selective drugs, which could be beneficial for particular indications, the reduction of undesirable side effects, or the targeting of therapeutics to selected tissues. Other purposes and advantages of the invention will be readily apparent to the artisan having ordinary skill in the art.

### SUMMARY OF THE INVENTION

It has now been discovered that these and other purposes can be achieved by the present invention, which, in one aspect, provides purified and isolated tankyrase2 polypeptides, preferably human tankyrase2 polypeptides. In particular the invention provides a purified and isolated tankyrase2 polypeptide comprising the amino acid sequence defined in SEQ ID NO:133 (designated “TANK2-LONG”) or SEQ ID NO:135 (designated “TANK2-SHORT”). The invention also provides polynucleotides encoding the tankyrase2 polypeptides. For example, the polynucleotide may comprise the coding region of the nucleotide sequence defined in SEQ ID NO:132 or SEQ ID NO:134.

The invention further provides polynucleotides that are complements to TANK2-encoding polynucleotides, as well as polynucleotides that hybridize under moderately stringent hybridization conditions to the coding or non-coding strand of the tankyrase2 polynucleotides. In a preferred case, the polynucleotide hybridizes to the complement of the polynucleotide defined in SEQ ID NO:132 or SEQ ID NO:134 under stringent hybridization conditions, and encodes a protein that: (a) has poly(ADP) polymerase activity, (b) interacts with damaged DNA, or (c) binds to telomere repeat-binding factors and/or modulates their activity.

The polynucleotides may be DNA molecules or RNA molecules. Certain desirable polynucleotides of the invention, e.g., oligonucleotide probes, may further comprise a detectable label moiety.

In another aspect, the invention provides an expression construct, comprising a tankyrase2-encoding polynucleotide, as well as host cells transformed or transfected with the expression constructs. The polynucleotide can be operatively linked to a heterologous promoter.

In a further aspect, the invention provides a method for producing a tankyrase2 polypeptide in a host cell modified to express the tankyrase polypeptide, comprising the steps of:

a) growing the host cell under conditions appropriate for expression of the tankyrase2 polypeptide; and

b) isolating the tankyrase2 polypeptide from the host cell or the medium in which the host cell is grown.

In yet another aspect, the invention provides antibodies that are immunoreactive with a tankyrase2 polypeptide. For example, the antibodies may be selected from the group consisting of monoclonal antibodies, polyclonal antibodies, single chain antibodies (scFv antibodies), chimeric antibodies, bifunctional/bispecific antibodies, humanized antibodies, human antibodies, CDR-grafted antibodies, Fab fragments, Fab′ fragments, F(ab′)₂ fragments, and Fv fragments. Also provided are cell lines that produce such antibodies. There are also provided anti-idiotype antibodies that are immunoreactive with tankyrase2-specific antibodies.

In still another aspect, the invention provides a method for identifying a binding partner of a tankyrase2 polypeptide, comprising:

a) contacting the tankyrase2 polypeptide with a test compound under conditions that permit binding of the tankyrase2 polypeptide and the test compound;

b) detecting binding of the test compound and the tankyrase2 polypeptide; and

c) identifying the test compound as a binding partner of the tankyrase2 polypeptide.

For example, the method can be used to identify binding partners that selectively or specifically modulate, i.e., inhibit or enhance, a biological activity of the tankyrase2 polypeptide.

Also provided in another aspect is a method for identifying a binding partner of a tankyrase2 polynucleotide, comprising:

a) contacting the tankyrase2 polynucleotide with a test compound under conditions that permit binding of the tankyrase2 polynucleotide and the test compound;

b) detecting binding of the test compound and the tankyrase2 polynucleotide; and

c) identifying the test compound as a binding partner of the tankyrase2 polynucleotide.

The method may be used to identify binding partners that selectively or specifically modulate, i.e., inhibit or enhance, expression of the tankyrase2 polypeptide.

There is also provided by the invention a method of treating a human or animal subject having a medical condition mediated by poly(ADP-ribose) polymerase activity, comprising administering to the subject a tankyrase2 inhibitory compound in an amount effective for inhibiting tankyrase2 in the subject. In another aspect, the invention provides a method of treating a human or animal subject having a medical condition mediated by poly(ADP-ribose) polymerase activity, comprising administering to the subject a compound that inhibits tankyrase2 expression or activity in an amount effective for inhibiting poly(ADP-ribose) polymerase activity in the subject. The method is of particular interest in treating medical conditions associated with growth of neoplastic tissue. For example, the method can be used to treat cancers such as carcinomas, sarcomas, leukemias, and lymphomas. More particularly, the method may be used to treat cancers selected from the group consisting of ACTH-producing tumor, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and non-small cell), malignant peritoneal effusion, malignant pleural effusion, melanoma, mesothelioma, multiple myeloma, neuroblastoma, glioma, non-Hodgkin's lymphoma, osteosarcoma, ovarian cancer, ovarian (germ cell) cancer, pancreatic cancer, penile cancer, prostate cancer, retinoblastoma, skin cancer, soft tissue sarcoma, squamous cell carcinomas, stomach cancer, testicular cancer, thyroid cancer, trophoblastic neoplasms, uterine cancer, vaginal cancer, cancer of the vulva, and Wilm's tumor.

These and other features and advantages of the present invention will be appreciated from the detailed description and examples that are set forth herein. The detailed description and examples are provided to enhance the understanding of the invention, but are not intended to limit the scope of the invention.

### DETAILED DESCRIPTION OF THE PREFERRED EMBODIMENTS

The present invention relates generally to a previously uncharacterized nucleic acid encoding a novel human protein designated “tankyrase2” (hereinafter also referred to as “TANK2”). As illustrated herein tankyrase2 is distinct from known tankyrase proteins and other proteins sharing poly(ADP-ribose) polymerase activity. The present invention is based on the discovery of novel gene encoding the tankyrase2 protein, and nucleic acid sequences, oligonucleotides, fragmnents, and antisense molecules thereof.

The nucleotide sequence information provided by the invention makes possible large-scale expression of the encoded TANK2 polypeptide by techniques well known and routinely practiced in the art. The invention also permits identification and isolation of polynucleotides encoding related TANK2 polypeptides by well-known techniques including Southern (DNA) and/or northern (mRNA) hybridization, and amplification techniques such as polymerase chain reaction (PCR), ligase chain reaction (LCR), and the like. Examples of related polynucleotides include human and non-human tank2 genomic sequences, including allelic variants, as well as polynucleotides encoding polypeptides homologous to TANK2 and structurally related polypeptides sharing one or more biological, immunological, and/or physical properties of TANK2.

The invention includes both naturally occurring and non-naturally occurring tankyrase2 polynucleotides and polypeptide products thereof. Naturally occurring tankyrase2 products include distinct polynucleotide and polypeptide tankyrase2 species as they occur in humans. However, the invention includes other human tankyrase2 polynucleotide and polypeptide species defined through the analysis of sequence homology. The invention further comprises corresponding homologs of human TANK2 polypeptides and tank2 polynucleotides that are expressed in cells of other animal species, preferably mammalian homologs, and more preferably primate homologs. Within each tankyrase2 species, the invention further provides splice variants, which are encoded by the same genomic DNA but arise from distinct mRNA transcripts. Non-naturally occurring tankyrase2 products include variants of the naturally occurring tankyrase2 products such as polynucleotide and polypeptide analogs (i.e., wherein one or more nucleotides or amino acids are added, substituted, or deleted). Non-naturally-occurring TANK2 polypeptide products also include TANK2 products that have been covalently modified, e.g., water-soluble polymer modifications, glycosylation variants, and the like.

The tankyrase2 polypeptides and the nucleic acids that encode the polypeptides provide a basis for diagnostic methods for the precise and accurate detection and/or quantitation of TANK2 expression and medical conditions associated with excessive or insufficient TANK2 activity. Furthermore, the nucleotide sequences disclosed herein may be used in the detection of aberrations, such as mutations and deletions, in the gene encoding TANK2. For example, the nucleotide sequences disclosed herein may be used to identify and isolate a genomic sequence for tank2. PCR primers can be designed from various portions of the introns and exons of a genomic tank2 nucleic acid sequence that will allow detection of aberrations in the genomic sequence.

The invention further provides methods of using TANK2 and genetically engineered host cells that express recombinant TANK2 to evaluate and screen for modulators of the poly(ADP-ribose) polymerase activity of the enzyme. Such screening methods may be used for the identification of allosteric agonists and antagonists of TANK2 activity as well as for the identification of direct (e.g., competitive inhibitors) of such activity. TANK2 protein antagonists and inhibitors, such as anti-TANK2 antibodies and tank2 antisense molecules, will provide the basis for pharmaceutical compositions for the treatment and amelioration of symptoms associated with excessive poly(ADP-ribose) polymerase activity. Agonists of TANK2 will provide the basis of the treatment and amelioration of symptoms associated with insufficient poly(ADP-ribose) polymerase activity.

Tankyrase2 Polynucleotides

The present invention provides, inter alia, novel purified and isolated polynucleotides encoding human TANK2 polypeptides. The polynucleotides of the invention include DNA sequences and RNA transcripts, both sense and complementary antisense strands, and splice variants thereof. DNA sequences of the invention include, without limitation. cDNA and genomic sequences. As used herein. lower case “tank2” refers to a tankyrase2 nucleic acid sequence whereas upper case “TANK2” refers to a tankyrase2 amino acid sequence.

“Nucleic acid” as used herein refers to an oligonucleotide or polynucleotide sequence, and fragments or portions thereof, and to DNA or RNA of genomic or synthetic origin, which may be double-stranded or single-stranded, whether representing the sense or antisense strand. An exemplary double-stranded polynucleotide according to the invention can have a first strand (i.e., a coding strand) having a sequence encoding a TANK2 polypeptide, along with a second strand (i.e., a “complementary” or “non-coding” strand) having a sequence deducible from the first strand according to the Watson-Crick base-pairing rules for DNA. Double-stranded or “duplex” structures may be DNA:DNA, DNA:RNA, or RNA:RNA nucleic acids. A preferred double-stranded polynucleotide is a cDNA comprising the coding region of a nucleotide sequence defined by SEQ ID NO: 132 or SEQ ID NO: 134. An exemplary single-stranded polynucleotide according to the invention is a messenger RNA (MRNA) encoding a TANK2 polypeptide. Another exemplary single-stranded polynucleotide is an oligonucleotide probe or primer that hybridizes to the coding or non-coding strand of a polynucleotide selected from among the sequences defined by SEQ ID NO:132, and SEQ ID NO:134. Other alternative nucleic acid structures, e.g., triplex structures, are also contemplated.

Genomic DNA of the invention comprises the protein-coding region for a TANK2 polypeptide and includes allelic variants of the preferred polynucleotides of the invention, such as single nucleotide polymorphisms. Genomic DNA of the invention is distinguishable from genomic DNAs encoding polypeptides other than TANK2 in that it includes the TANK2-coding region found in tank2 cDNA of the invention. Genomic DNA can be transcribed into RNA, and the resulting RNA transcript may undergo one or more splicing events wherein one or more introns (i.e., non-coding regions) of the transcript are removed, or “spliced out.” RNA transcripts that can be spliced by alternative mechanisms and therefore be subjected to removal of different non-coding RNA sequences but still encode a TANK2 polypeptide, are referred to in the art as “splice variants,” and are embraced by the invention Splice variants comprehended by the invention, therefore, are encoded by the same DNA sequences but give rise to different amino acid sequences. Such splice variants can comprise regions in which the reading frame is shifted, wherein a downstream portion of the RNA sequence is translated differently, to yield different amino acid sequences in the resulting polypeptides. Allelic variants are known in the art to be modified forms of the wild-type (predominant) gene sequence. Such modifications result from recombination during chromosomal segregation or exposure to conditions that give rise to genetic mutation. Allelic variants, like wild-type genes, are naturally occurring sequences, as opposed to non-naturally occurring variants, which arise from in vitro manipulation.

The invention also comprehends cDNA, which is obtained through reverse transcription of an RNA polynucleotide encoding TANK2 followed by second strand synthesis of a complementary strand to provide a double stranded DNA. For example, the invention provides a cDNA sequence that encodes a polypeptide having an amino acid sequence selected from among the sequences defined by SEQ ID NO:133 and SEQ ID NO:135. In a preferred embodiment, the invention provides polynucleotides comprising the coding region of a nucleotide sequence selected from among the sequences defined by SEQ ID NO:132 and SEQ ID NO:134.

As noted, highly preferred nucleic acid sequences according to the invention are defined by SEQ ID NO:132 or SEQ ID NO:134. However, because the genetic code is redundant or “degenerate” in its information-encoding properties, different nucleotide sequences may encode the same polypeptide sequence. Accordingly, the invention comprises the alternative (degenerate) nucleotide sequences that encode TANK2 polypeptides of the invention and functional equivalents thereof. For example, the invention includes polynucleotides comprising nucleotide sequences that are substantially homologous to the TANK2-encoding regions of the nucleotide sequences set forth in SEQ ID NO:132 or SEQ ID NO:134. More particularly, the invention includes polynucleotides whose corresponding nucleotide sequences have at least 90%, preferably at least 95%, more preferably at least 98%, and still more preferably at least 99% identity with a nucleotide sequence defined in SEQ ID NO:132 or SEQ ID NO:134.

Variant polynucleotides of the invention further include fragments of the tank2 nucleotide sequences defined in SEQ ID NO:132 and SEQ ID NO:134, and homologs thereof. The disclosure of full-length polynucleotides encoding TANK2 polypeptides makes readily available to the person having ordinary skill in the art every possible fragment of the full-length polynucleotides. Preferably, fragment polynucleotides of the invention comprise sequences unique to the TANK2-coding nucleotide sequence, and therefore hybridize under highly stringent or moderately stringent conditions only (i.e., specifically) to polynucleotides encoding TANK2 or fragments thereof containing the unique sequence. Polynucleotide fragments of genomic sequences of the invention comprise not only sequences unique to the coding region, but also include fragments of the full-length sequence derived from introns, regulatory regions, and/or other untranslated sequences. Sequences unique to polynucleotides of the invention are recognizable through sequence comparison to other known polynucleotides, and can be identified through use of computer software routinely used in the art, e.g., alignment programs available in public sequence databases.

The invention also provides fragment polynucleotides that are conserved in one or more polynucleotides encoding members of the TANK2 family of polypeptides. Such fragments include sequences characteristic of the family of TANK2 polypeptides, referred to as “signature” sequences. The conserved signature sequences are readily discernable following simple sequence comparison of polynucleotides encoding members of the TANK2 family. Polynucleotide fragments of the invention can be labeled in a manner that permits their detection, including radioactive and non-radioactive labeling.

Hybridization can be defined to include the process of forming partially or completely double-stranded nucleic acid molecules through sequence-specific association of complementary single-stranded nucleic molecules. The invention, therefore, further encompasses the use of nucleic acid species that hybridize to the coding or non-coding strands of a polynucleotide that encodes a TANK2 protein. Preferred hybridizing species hybridize to the coding or non-coding strand of the nucleotide sequence defined by SEQ ID NO:132 or SEQ ID NO:134. Also encompassed are species that would hybridize to a TANK2-encoding polynucleotide but for the redundancy of the genetic code, i.e., polynucleotides that encode the same amino acid sequence but rely on different codon usage.

Hybridizing species include, for example, nucleic acid hybridization or amplification probes (oligonucleotides) that are capable of detecting nucleotide sequences (e.g., genomic sequences) encoding TANK2 or closely related molecules, such as alleles. The specificity of the probe, i.e., whether it is derived from a highly conserved, conserved, or non-conserved region or domain. and the stringency of the hybridization or amplification conditions (high, intermediate, or low) will determine whether the probe identifies only naturally occurring tank2, or related sequences. Probes for the detection of related nucleotide sequences are selected from conserved or highly conserved regions of tank2 family members and such probes may be used in a pool of degenerate probes. For the detection of identical nucleotide sequences, or where maximum specificity is desired, oligonucleotide probes are selected from the non-conserved nucleotide regions or unique regions of tank2 polynucleotides. As used herein, the term “non-conserved nucleotide region” refers to a nucleotide region that is unique to tank2 disclosed herein and does not occur in related tank2 family members.

Specificity of hybridization is typically characterized in terms of the degree of stringency of the conditions under which the hybridization is performed. The degree of stringency of hybridization conditions can refer to the melting temperature (Tₘ) of the nucleic acid binding complex [see, e.g., Berger and Kimmel, “Guide to Molecular Cloning Techniques,” Methods in Enzymology, Vol. 152, Academic Press, San Diego, Calif. (1987)]. “Maximal stringency” typically occurs at about Tₘ−5° C. (5° C. below the Tₘ of the probe); “high stringency” at about 5° C. to 10° C. below Tₘ; “intermediate stringency” at about 10° C. to 20° C. below Tₘ; and “low stringency” at about 20° C. to 25° C. below Tₘ.

Alternatively, the stringency of hybridization can refer to the physicochemical conditions employed in the procedure. To illustrate, exemplary moderately stringent hybridization conditions are: hybridization in 3×saline sodium citrate (SSC), 0.1% sarkosyl, and 20 mM sodium phosphate, pH 6.8, at 65° C.; and washing in 2×SSC with 0.1% sodium dodecyl sulfate (SDS), at 65° C. Exemplary highly stringent hybridization conditions are: hybridization in 50% formamide, 5×SSC, at 42° C. overnight, and washing in 0.5×SSC and 0.1% SDS, at 50° C. It is understood in the art that conditions of equivalent stringency can be achieved through variation of temperature and buffer, or salt concentration as described Ausubel et al. (Eds.), Current Protocols in Molecular Biology, John Wiley & Sons (1994), at pp. 6.0.3-6.4.10. Modifications in hybridization conditions can be determined empirically or calculated precisely based on the length of the oligonucleotide probe and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, N.Y. (1989), pp. 9.47-9.51.

The artisan will appreciate that hybridization under more stringent conditions enables the identification of species having a higher degree of homology or sequence identity with the target sequence. By contrast, hybridization under less stringent conditions enables identification of species having a lesser but still significant degree of homology or sequence identity with the target sequence. Therefore, also included within the scope of the present invention are nucleic acid species that are capable of hybridizing to the nucleotide sequence of SEQ ID NO:132 or SEQ ID NO:134 under conditions of intermediate (moderate) to maximal stringency. Preferably, the hybridizing species hybridize to the coding or non-coding strands of a polynucleotide defined by SEQ ID NO:132 or SEQ ID NO:134 under highly stringent conditions.

The polynucleotides of the invention encompass oligonucleotides (i.e., nucleic acid oligomers typically about 10 to 60 nucleotides in length) that hybridize to either the coding or the non-coding strands of a nucleic acid encoding a TANK2 amino acid sequence. In particular, the invention comprises oligonucleotides that hybridize to the coding or non-coding strand of a polynucleotide defined by SEQ ID NO:132 or SEQ ID NO:134. The length of the oligonucleotide is not critical, as long as it is capable of hybridizing to the target nucleic acid molecule. However, longer nucleic acid molecules are more difficult to prepare and require longer hybridization times. Therefore, the oligonucleotide should not be longer than necessary. Accordingly, the oligonucleotide should contain at least 10 nucleotides, preferably at least 15 nucleotides, and more preferably at least 20 nucleotides. Nomially, the oligonucleotide will not contain more than 60 nucleotides, preferably not more than 30 nucleotides, and more preferably not more than 25 nucleotides. Such oligonucleotides may be used as described herein as primers for DNA synthesis (e.g., as primers in PCR; “amplimers”), as probes for detecting the presence of target DNA in a sample (e.g., northern or Southern blots and in situ hybridization), as therapeutic agents (e.g., in antisense therapy), or for other purposes. Oligonucleotides may be single- or double-stranded, with the double-stranded forms having one or both ends blunt or stepped.

The oligonucleotides may be obtained or derived by known methods from natural sources. Alternatively, the oligonucleotides may be produced synthetically according to methods known in the art. Such methods include, for example, cloning and restriction of appropriate sequences or direct chemical synthesis by any suitable method. Various chemical methods for making oligonucleotides are known in the art, including the phosphotriester method, the phosphodiester method; the diethylphosphoramidite method; the solid support method, and the H-phosphonate method [for reviews, see Caruthers, Science 230:281-5 (1985); Caruthers et al., Methods Enzymol 211:3-20 (1992)]. Typically, preparation of oligonucleotides is carried out by automated phosphoramidite synthesis on polymer support. Nucleic acid molecules consisting of 100 or more nucleotides may also be produced by such methods.

The tank2 polynucleotides of the invention include variants, which are polynucleotides that encode hAPRP2 or a functional equivalent thereof, and which can include deletions, insertions, or substitutions of nucleotide residues. As used herein a “deletion” is a change in a nucleotide or amino acid sequence in which one or more nucleotides or amino acid residues, respectively, are absent. As used herein an “insertion” or “addition” is a change in a nucleotide or amino acid sequence that results in the addition of one or more nucleotides or amino acid residues, respectively. As used herein a “substitution” is a change in a nucleotide or amino acid sequence in which one or more nucleotides or amino acids are replaced by different nucleotides or amino acids, respectively.

Polynucleotide variants also included within the scope of the present invention are alleles or alternative naturally occurring forms of tank2. Alleles result from naturally occurring mutations, i.e., deletions, insertions or substitutions, in the genomic nucleotide sequence, which may or may not alter the structure or function or the expressed polypeptides. Each of these types of mutational changes may occur alone, or in combination with the others, one or more times in a given allelic sequence. Single nucleotide polymorphisms (SNPs) may occur, in which a single base mutation may define an altered polypeptide, which in turn may be associated with an overt phenotypic difference. Of course, SNPs may be silent, as they may not change the encoded polypeptide, or any change they do encode may have no effect on phenotype.

The invention further embraces natural homologs of the human tankyrase2 DNA that occur in other animal species, such as other mammal species. Mammalian homologs include, for example, homologs in mouse, rat, guinea pig, and the like, and more preferably homologs in other primate species. Such species homologs, in general, share significant homology at the nucleotide level within the protein-coding regions. Thus, the invention encompasses polynucleotides that share at least 75%, at least 80%, at least 85%, at least 90%, at least 95%, at least 98%, or at least 99% nucleotide identity with the protein-coding region of a polynucleotide encoding a human TANK2 polypeptide, e.g., a polynucleotide defined by SEQ ID NO:132 or SEQ ID NO:134. Percent sequence “homology” with respect to polynucleotides of the invention can be defined as the percentage of nucleotide bases in a candidate sequence that are identical to nucleotides in the TANK2-encoding sequence after aligning the sequences and introducing gaps, if necessary, to achieve maximum percent sequence identity. Computer software is available (from commercial and public domain sources) for calculating percent identity in an automated fashion (e.g., FASTA).

The invention includes polynucleotides that have been engineered to selectively modify the cloning, processing, and/or expression of the TANK2 gene product. Mutations may be introduced using techniques well known in the art, e.g., site-directed mutagenesis to insert new restriction sites, to alter glycosylation patterns, or to change codon preferences inherent in the use of certain expression systems. while simultaneously maintaining control of the amino acid sequence of the expressed polypeptide product. For example, codons preferred by a particular prokaryotic or eukaryotic host cell can be selected (“codon optimization”) to increase the rate of TANK2 expression or to produce recombinant RNA transcripts having desirable properties, such as longer half-lives.

The tank2 polynucleotides can be synthesized, wholly or partly, using chemical methods well known in the art. “Chemically synthesized,” as used herein and is understood in the art, refers to purely chemical, as opposed to enzymatic, methods for producing polynucleotides. “Wholly” chemically synthesized DNA sequences are therefore produced entirely by chemical means; “partly” chemically synthesized DNAs embrace those wherein only portions of the resulting DNA were produced by chemical means.

DNA molecules may be modified to increase intracellular stability and half-life. Possible modifications include, but are not limited to, the addition of flanking sequences of the 5′ and/or 3′ ends of the molecule or the use of phosphorothioate or 2′ O-methyl rather than phosphodiester linkages within the backbone of the molecule.

The invention also provides TANK2 peptide nucleic acid (PNA) molecules. These TANK2 PNAs are informational molecules that have a neutral “peptide-like” backbone with nucleobases that allow the molecules to hybridize to complementary TANK2-encoding DNA or RNA with higher affinity and specificity than corresponding oligonucleotides (PerSeptive Biosystems).

Polypeptide Expression Systems

Knowledge of TANK2-encoding DNA sequences enables the artisan to modify cells to permit or increase expression of TANK2. Accordingly, host cells are provided, including prokaryotic or eukaryotic cells, either stably or transiently modified by introduction of a polynucleotide of the invention to permit expression of the encoded TANK2 polypeptide. Autonomously replicating recombinant expression constructs such as plasmid and viral DNA vectors incorporating TANK2-encoding sequences are also provided.

Expression constructs are also provided comprising TANK2-encoding polynucleotides operatively linked to an endogenous or exogenous expression control DNA sequence and a transcription terminator. Expression control DNA sequences include promoters, enhancers, and operators, and are generally selected based on the expression systems in which the expression construct is to be used. Preferred promoter and enhancer sequences are generally selected for the ability to increase gene expression, while operator sequences are generally selected for the ability to regulate gene expression. Preferred constructs of the invention also include sequences necessary for replication in a host cell. Expression constructs are preferably used for production of an encoded TANK2 polypeptide, but may also be used to amplify the construct itself.

Polynucleotides of the invention may be introduced into the host cell as part of a circular plasmid, or as linear DNA comprising an isolated protein coding region or a viral vector. Methods for introducing DNA in to a host cell include transformation, transfection, electroporation, nuclear injection, or fusion with carriers such as liposomes, micelles, ghost cells, and protoplasts. Expression systems of the invention include, for example, bacteria, yeast, fungal, plant, insect, invertebrate, amphibian, and mammalian cell systems. Some suitable prokaryotic host cells include, for example, E. coli strains SG-936, HB 101, W3110, X 1776, X2282, DHI, and MRC1, Pseudomonas sp., Bacillus sp. such as B. subtilis, and Streptomyces sp. Suitable eukaryotic host cells include yeasts, such as Saccharomyces cerevisiae, S. pombe, Pichia pastoris and other fungi, insect cells such as sf9 or sf21 cells (Spodoptera frugiperda), animal cells such as Chinese hamster ovary (CHO) cells, human cells such as JY, 293, and NIH3T3 cells, and plant cells such as Arabidopsis thaliana cells. The tank2 nucleotide sequence, or any portion of it, may be cloned into a vector for the production of an mRNA probe. Such vectors are known in the art, are commercially available, and may be used to synthesize RNA probes in vitro by addition of labeled nucleotides and an appropriate RNA polymerase such as T7, T3, or SP6.

The type of host cell, the form of the expressed TANK2 product, the conditions of growth, etc., can be selected by the skilled artisan according to known criteria. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of TANK2 polypeptides are embraced. The protein produced by a recombinant cell may be secreted or may be contained intracellularly, depending on the sequence and/or the vector used. As will be understood by those of skill in the art, expression vectors containing tank2 can be designed with signal sequences that direct secretion of TANK2 through a particular prokaryotic or eukaryotic cell membrane.

Expression constructs may include sequences that facilitate, and preferably promote, homologous recombination in a host cell. This can be accomplished by replacing all or part of the naturally occurring tank2 promoter with all or part of a heterologous promoter so that the cells express TANK2 at higher levels. The heterologous promoter should be inserted so that it is operatively linked to TANK2-encoding sequences. See, for example, PCT International Publication Nos. WO 94/12650, WO 92/20808, and WO 91/09955.

Host cells of the invention are useful in methods for large-scale production of TANK2 polypeptide products. For example, host cells of the invention are a valuable source of immunogen for development of antibodies that are immunoreactive with TANK2 polypeptides. As another example, recombinant TANK2 can be produced and isolate from host cells for use in in vitro binding assays such as drug screening assays. In such methods, the host cells are grown in a suitable culture medium and the desired polypeptide product is isolated from the cells or from the medium in which the cells are grown.

The polypeptide product can be isolated by purification methods known in the art, such as conventional chromatographic methods including immunoaffinity chromatography, receptor affinity chromatography, hydrophobic interaction chromatography, lectin affinity chromatography, size exclusion filtration, cation or anion exchange chromatography, high performance liquid chromatography (HPLC), reverse phase HPLC, and the like.

Still other methods of purification include those in which the desired protein is expressed and purified as a fusion protein in which the TANK2 polypeptide is ligated to a heterologous amino acid sequence. Suitable heterologous sequences can include a specific tag, label, or chelating moiety that is recognized by a specific binding partner or agent. For example, for screening of peptide libraries for modulators of TANY2 activity, it is possible to express a TANK2 protein fused to a selected heterologous protein selected to be specifically identifiable using a probe antibody. A fusion protein may also be engineered to contain a cleavage site (e.g., a factor XA or enterokinase sensitive sequence) located between the TANK2 sequence and the heterologous protein sequence, to permit the TANK2 protein to be cleaved from the heterologous protein and subsequently purified. Cleavage of the fusion component may produce a form of the desired protein having additional amino acid residues resulting from the cleavage process.

Exemplary heterologous peptide domains include metal-chelating peptides such as histidine-tryptophan modules that allow purification on immobilized metals [Porath, Protein Expr Purif 3:263-81 (1992)], and protein A domains that allow purification on immobilized immunoglobulin. Another useful system is the divalent cation-binding domain and antibodies specific thereto used in the peptide extension/immunoaffinity purification system described in U.S. Pat. Nos. 4,703,004; 4,782,137; 4,851,431; and 5,011,912. This system is commercially available as the FLAG® system from Immunex Corp. (Seattle Wash.). Another suitable heterologous fusion partner is glutathione S-transferase (GST), which can be affinity purified using immobilized glutathione. Other useful fusion partners include immunoglobulins and fragments thereof, e.g., Fc fragments.

Identification of host cells expressing recombinant TANK2 may be crucial to identifying appropriate expression systems. Accordingly, expression constructs of the invention may also include sequences encoding one or more selectable markers that permit identification of host cells bearing the construct in operative condition. It is also contemplated that, in addition to the insertion of heterologous promoter DNA, amplifiable marker DNA (e.g., ada, dhfr, and the multifunctional CAD gene that encodes carbamyl phosphate synthase, aspartate transcarbamylase, and dihydroorotase) and/or intron DNA may be inserted along with the heterologous promoter DNA. If linked to the TANK2-encoding sequence, amplification of the marker DNA by standard selection methods results in co-amplification of the TANK-2-encoding sequences in the cells. Detection of expression of the marker gene in response to induction or selection usually indicates expression of TANK2 as well. Alternatively, if the tank2 polynucleotide is inserted within a marker gene sequence, recombinant cells containing tank2 can be identified by the absence of marker gene function.

Host cells that contain the coding sequence for TANK2 and express TANK2 may also be identified by a variety of other procedures known to those of skill in the art. These procedures include, but are not limited to, DNA-DNA or DNA-RNA hybridization and protein bioassay or immunoassay techniques that include membrane-based, solution-based, or chip-based technologies for the detection and/or quantification of the nucleic acid or protein.

The presence of the tank2 polynucleotide sequence can be detected by DNA-DNA or DNA-RNA hybridization or amplification using fragments of a tank2 polynucleotide, e.g., fragments of the sequences set forth in SEQ ID NO:132 or SEQ ID NO:134, as probes. Nucleic acid amplification based assays involve the use of oligonucleotides based on the tank2 sequence to detect transformants containing tank2 DNA or RNA. Labeled hybridization or PCR probes for detecting tank2 polynucleotide sequences can be made by various methods, including oligolabeling, nick translation, end-labeling, or PCR amplification using a labeled nucleotide. In an embodiment of the present invention, TANK2 or a variant thereof and/or a host cell line that expresses the TANK2 or variant thereof may be used to screen for antibodies, peptides, or other molecules, such as organic or inorganic molecules, that act as modulators of a biological or immunological activity of TANK2. For example, anti-TANK2 antibodies capable of neutralizing the polymerase or DNA-binding activity of TANK2 may be used to inhibit TANK2-mediated cell death. Alternatively, screening of peptide libraries or organic libraries made by combinatorial chemistry with recombinantly expressed TANK2 or variants thereof or cell lines expressing TANK2 or variants thereof may be useful for identification of therapeutic molecules that function by modulating a biological or immunological activity of TANK2. Synthetic compounds, natural products, and other sources of potentially biologically active materials can be screened in a number of ways deemed routine by those of skill in the art. For example, nucleotide sequences encoding the DNA-binding domain of TANK2 may be expressed in a host cell, which can be used for screening of allosteric modulators, either agonists or antagonists, of TANK2 activity. Alternatively, nucleotide sequences encoding the conserved catalytic domain of TANK2 can be expressed in host cells and used to screen for inhibitors of ADP-ribose polymerization.

TANK2 Polypeptides

The invention also provides purified and isolated mammalian TANK2 polypeptides. Exemplary TANK2 polypeptides have amino acid sequences defined in SEQ ID NO:133 or SEQ ID NO:135. TANK2 polypeptides of the invention may be isolated from natural cell sources or may be chemically synthesized, but are preferably produced by recombinant procedures involving host cells of the invention. TANK2 products of the invention may be full-length polypeptides, or variant polypeptide products such as fragments, truncates, deletion mutants, and other variants thereof that retain specific TANK2 biological activity. As used herein, “biologically active” refers to a TANK2 polypeptide having structural, regulatory or biochemical functions of the naturally occurring TANK2 protein. Specifically, a TANK2 protein of the present invention has the ability to bind DNA and to polymerize ADP-ribose subunits in response to DNA damage in a cell.

The protein and fragments of the present invention may be prepared by methods known in the art. Such methods include isolating the protein directly from cells, isolating or synthesizing DNA encoding the protein and using the DNA to produce recombinant protein, and synthesizing the protein chemically from individual amino acids.

The TANK2 polypeptides can be isolated from a biological sample, such as a solubilized cell fraction, by standard methods. Some suitable methods include precipitation and liquid chromatographic protocols such as ion exchange, hydrophobic interaction, and gel filtration [see, e.g.. Deutscher (Ed.), Methods Enzymol (Guide to Protein Chemistry, Section VII) 182:309 (1990) and Scopes, Protein Purification. Springer-Verlag, New York (1987)]. Alternatively, purified material is obtained by separating the protein on preparative SDS-PAGE gels, slicing out the band of interest and electroeluting the protein from the polyacrylamide matrix by methods known in the art. The detergent SDS is removed from the protein by known methods, such as by dialysis or the use of a suitable column, such as the Extracti-Gel® column from Pierce Chemical Co. (Rockford, Ill.).

The TANK2 polypeptide of the invention may also be chemically synthesized, wholly or partly, by methods known in the art [see, e.g., Stuart and Young, Solid Phase Peptide Synthesis, 2d ed., Pierce Chemical Co. (1984)]. For example, peptides can be synthesized by solid phase techniques, cleaved from the resin, and purified by preparative HPLC [see, e.g., Roberge et al., Science 269:202-4 (1995)]. Automated synthesis may be accomplished, for example, using the ABI 431A Peptide Synthesizer (Perkin Elmer, Norwalk, Conn.) in accordance with the instructions provided by the manufacturer. The composition of the synthetic peptides may be confirmed by amino acid analysis or sequencing (e.g., the Edman degradation procedure).

Recombinant TANK2 protein may be produced in and isolated from a host cell transformed with an expression vector containing a tank2 nucleotide sequence and grown in cell culture. As described herein, the host cells, either prokaryotic or eukaryotic, are either stably or transiently transfected (eukaryotic) or transformed (prokaryotic) with a TANK2-encoding polynucleotide of the invention in manner that permits directed expression of a TANK2 polypeptide. In such methods, the host cells are grown in a suitable culture medium and the desired polypeptide products are isolated from the cells or from the medium in which the cells are grown. Isolation of the polypeptides can be accomplished by, for example, immunoaffinity purification. The use of transformed host cells is preferred for large-scale production of TANK2 polypeptides.

The invention includes polypeptides comprising amino acid sequences that are substantially homologous to the sequences of TANK2 polypeptides described herein. For example, the invention includes polypeptides whose corresponding amino acid sequences have at least 90%, preferably at least 95%. more preferably at least 98%. and still more preferably at least 99% identity with the polypeptide sequence defined in SEQ ID NO:133 or SEQ ID NO:135.

Percent sequence “identity” with respect to a preferred polypeptide of the invention can be defined as the percentage of amino acid residues in a candidate sequence that are identical to amino acid residues in the reference TANK2 sequence after aligning the sequences and introducing gaps, if necessary, to achieve maximum percent sequence identity, and not considering any conservative substitutions as part of the sequence identity.

Percent sequence “homology” with respect to a preferred polypeptide of the invention can be defined as the percentage of amino acid residues in a candidate sequence that are identical to amino acid residues in the reference TANK2 sequence after aligning the sequences and introducing gaps, if necessary, to achieve maximum percent sequence identity, and also considering any conservative substitutions as part of the sequence identity.

Determinations of whether two amino acid sequences are substantially homologous can also be based on FASTA searches [Pearson et al., Proc Natl Acad Sci USA 85:2444-8 (1988)]. Alternatively, percent homology is calculated as the percentage of amino acid residues in the smaller of the two sequences that align with identical amino acid residues in the sequence being compared, when four gaps in a length of 100 amino acids may be introduced to maximize alignment [see Dayhoff, in Atlas of Protein Sequence and Structure, Vol. 5, National Biochemical Research Foundation, Washington, D.C. (1972), at p. 124].

A polypeptide may be considered homologous to a TANK2 polypeptide of the invention if polynucleotides encoding the two polypeptides hybridize with one another. A higher degree of homology is shown if the hybridization occurs under hybridizationf-conditions of greater stringency. Control of hybridization conditions and the relationships between hybridization conditions and degree of homology are understood by those skilled in the art [see, e.g., Sambrook et al., supra]. Thus, a homologous polypeptide may be a polypeptide that is encoded by a polynucleotlde that hybridizes with a polynucleotide encoding a polypeptide of the invention under hybridization conditions having a specified degree of stringency.

It may be desirable that such structurally homologous polypeptides will also exhibit functional homology, insofar as the homologous polypeptide has substantially the same function as the polypeptide of the invention. For example, structurally homologous polypeptides may be considered functionally homologous if they exhibit similar immune reactivity, etc.

However, it is known that two polypeptides or two polynucleotides may be considered to be substantially homologous in structure, and yet differ substantially in function. For example, single nucleotide polymorphisms (SNPs) among alleles may be expressed as polypeptides having substantial differences in function along one or more measurable parameters such as antibody- or ligand-binding affinity or enzymatic substrate specificity, and the like. Other structural differences, such as substitutions, deletions, splicing variants, and the like, may affect the function of otherwise structurally identical or homologous polypeptides.

The TANK2 polypeptides of the invention include functional derivatives of a TANK2 polypeptides defined in SEQ ID NO:133 or SEQ ID NO:135. Such functional derivatives include polypeptide products that possesses a structural feature or a biological activity that is substantially similar to a structural feature or a biological activity of the TANK2 protein. Accordingly, functional derivatives include variants, fragments, and chemical derivatives of the parent TANK2 protein.

As used herein “variant” refers to a molecule substantially similar in structure and function to either the entire TANK2 molecule, or to a fragment thereof. A molecule is said to be “substantially similar” to another, if both molecules have substantially similar structures or if both molecules possess a similar biological activity. Thus, provided that two molecules possess a similar activity, they are considered variants, as that term is used herein, even if one of the molecules possesses a structure not found in the other molecule, or if the sequence of amino acid residues is not identical.

Among the variant polypeptides provided under the invention are variants that comprise one or more changes in the amino acid sequence of the TANK2 polypeptide. Such sequence-based changes include deletions, substitutions or insertions in the TANK2 sequence, as well as combinations thereof.

Deletion variants of the TANK2 polypeptides are polypeptides in which at least one amino acid residue of the sequence is removed. Deletions can be effected at one or both termini of the protein, or with removal of one or more residues within the TANK2 amino acid sequence. Deletion variants include, for example, all incomplete fragments of the TANK2 polypeptides of the invention. As used herein “fragment” refers to any polypeptide subset of the TANK2 protein.

Fragments of TANK2 that exhibit a biological activity characteristic of TANK2 and that are soluble (i.e., not membrane bound) are desirable. A soluble fragment is preferably generated by deleting any membrane-spanning region(s) of the parent molecule or by deleting or substituting hydrophilic amino acid residues for hydrophobic residues. Identification of such residues is well known in the art.

Substitution variants are provided, including polypeptides in which at least one amino acid residue of a TANK2 polypeptide is replaced by an alternative residue. Any substitution can be made, with conservative substitutions being preferred. Directed amino acid substitutions may be made based on well defined physicochemical parameters of the canonical and other amino acids (e.g., the size, shape, polarity, charge, hydrogen-bonding capacity, solubility, chemical reactivity, hydrophobicity, hydrophilicity, or the amphipathic character of the residues.) as well as their contribution to secondary and tertiary protein structure. Substitution variants can include polypeptides comprising one or more conservative amino acid substitutions, i.e., a substitution of one amino acid by another having similar physicochemical character as desired. To illustrate, the canonical amino acids can be grouped according to the following categories:

| | |
|---|---|
| Aliphatic Side Chains | Gly, Ala; Val, Leu, Ile |
| Aromatic Side Chains | Phe, Tyr, Trp |
| Aliphatic Hydroxyl Side Chains | Ser, Thr |
| Basic Side Chains | Lys, Arg, His |
| Acidic Side Chains | Asp, Glu |
| Amide Side Chains | Asn, Gln |
| Sulfur-Containing Side Chains | Cys, Met |
| Secondary Amino Group | Pro |

Substitutions are preferably made in accordance with the following Table 1 when it is desired to controllably define the characteristics of the TANK2 molecule.

TABLE 1

| | Exemplary Conservative |
|---|---|
| Original Residue | Substitutions |
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | leu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Substantial changes in functional or immunological identity are made by selecting substitutions that are more progressive than those in Table 1, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that are in general more progressive are those in which: (a) glycine and/or proline is substituted by another amino acid or is deleted or inserted; (b) a hydrophilic residue is substituted for a hydrophobic residue; (c) a cysteine residue is substituted for (or by) any other residue; (d) a residue having an electropositive side chain is substituted for (or by) a residue having an electronegative charge; or (e) a residue having a bulky side chain is substituted for (or by) one not having such a side chain. Most preferred are amino acid substitutions that affect the solubility of TANK2. These are most preferably generated by substituting hydrophilic for hydrophobic amino acids.

Substitution variants, however, can include non-canonical or non-naturally occurring amino acid residues substituted for amino acid residues in the principal sequence. Substitution variants include those polypeptides in which amino acid substitutions have been introduced by modification of polynucleotides encoding a TANK2 polypeptide.

Insertion variants are provided, in which at least one amino acid residue is present in addition to a TANK2 amino acid sequence. Insertions may be located at either or both termini of the polypeptide, or may be positioned within the TANK2 amino acid sequence. Insertional variants also include fusion proteins in which the amino or carboxy terminus of the TANK2 polypeptide is fused to another polypeptide. Examples of such fusion proteins include immunogenic polypeptides, proteins with long circulating half-life (e.g., immunoglobulin constant regions), marker proteins (e.g., green fluorescent protein) and proteins or polypeptides that facilitate purification of the desired TANK2 polypeptide (e.g., FLAG® tags or polyhistidine sequences). Another example of a terminal insertion is a fusion of a signal sequence, whether heterologous or homologous to the host cell, to the N-terminus of the molecule to facilitate the secretion of the derivative from recombinant hosts. Intrasequence insertions (i.e., insertions within a TANK2 molecule sequence) may range generally from about 1 to 10 residues, more preferably 1 to 5.

Polypeptide variants of the invention also include mature TANK2 products, i.e., TANK2 products wherein leader or signal sequences are removed, as well as products having additional amino terminal residues. TANK2 products having an additional methionine residue at position-1 (Met⁻³-TANK2) are contemplated, as are TANK2 products having additional methionine and lysine residues at positions -2 and -1, respectively (Met⁻²-Lys⁻¹-TANK2). Other such variants are particularly useful for recombinant protein production in bacterial host cells.

The invention also encompasses TANK-2 variants having additional amino acid residues resulting from use of specific expression systems. For example, use of commercially available vectors that express a desired polypeptide as a glutathione-S-transferase (GST) fusion product yields the desired polypeptide having an additional glycine residue at position -1 (Gly⁻¹-TANK2) upon cleavage of the GST component from the desired polypeptide. Variants that result from expression in other vector systems are also contemplated.

The invention further provides TANK2 polypeptide products that are chemical derivatives of a TANK2 polypeptide defined in SEQ ID NO:133 or SEQ ID NO:135. As used herein, the term “chemical derivative” refers to molecules that contain additional chemical moieties that are not normally a part of the naturally occurring molecule. Such moieties may impart desirable properties to the derivative molecule, such as increased solubility, absorption, biological half-life, etc. The moieties may alternatively decrease the toxicity of the derivative molecule, or eliminate or attenuate any undesirable side effect of the derivative molecule. Thus, chemical derivatives of TANK2 polypeptides include polypeptides bearing modifications other than (or in addition to) insertion, deletion or substitution of amino acid residues. Preferably, the modifications are covalent in nature, and include, for example, chemical bonding with polymers, lipids, non-naturally occurring amino acids, and other organic and inorganic moieties. Derivatives of the invention may be prepared to increase circulating half-life of a TANK2 polypeptide, or may be designed to improve targeting capacity for the polypeptide to desired cells, tissues, or organs.

For example, methods are known in the art for modifying a polypeptide to include one or more water-soluble polymer attachments such as polyethylene glycol, polyoxyethylene glycol, or polypropylene glycol. Particularly preferred are TANK2 products that have been covalently modified with polyethylene glycol (PEG) subunits. Water-soluble polymers may be bonded at specific positions, for example at the amino terminus of the TANK2 products, or randomly attached to one or more side chains of the polypeptide. Additional derivatives include TANK2 species immobilized on a solid support, pin microparticle, or chromatographic resin. as well as TANK2 species modified to include one or more detectable labels. tags, chelating agents, and the like.

Derivatization with bifunctional agents can be used to cross-link TANKS to a water-insoluble support matrix. Alternatively, reactive water-insoluble matrices such as cyanogen bromide-activated carbohydrates and reactive substrates may be employed for protein immobilization [see, e.g., U.S. Pat. Nos. 3,969,287; 3,691,016; 4,195,128; 4,247,642; 4,229,537; and 4,330,440.]

Expression of TANK2 variants can be expected to have utility in investigating a biological activity characteristic of a wild-type TANK2 polypeptide. TANK2 variants can be designed to retain all biological or immunological properties characteristic for TANK2, or to specifically disable one or more particular biological or immunological properties of TANK2. For example, fragments and truncates may be designed to delete a domain associated with a particular property, or substitutions and deletions may be designed to inactivate a property associated with a particular domain. Forced expression (overexpression) of such variants (“dominant negative” mutants) can be employed to study the function of the protein in vivo by observing the phenotype associated with the mutant.

Functional derivatives of TANK2 having up to about 100 residues may be conveniently prepared by in vitro synthesis. If desired, such fragments may be modified using methods known in the art by reacting targeted amino acid residues of the purified or crude protein with an organic derivatizing agent that is capable of reacting with selected side chains or terminal residues. The resulting covalent derivatives may be used to identify residues important for biological activity.

Functional derivatives of TANK2 having altered amino acid sequences can also be prepared by mutating the DNA encoding TANK2. Any combination of amino acid deletion, insertion, and substitution may be employed to generate the final construct, provided that the final construct possesses the desired activity. Obviously. the mutations that will be made in the DNA encoding the functional derivative must not place the sequence out of reading frame and preferably will not create complementary regions that could produce secondary mRNA structure [see EP Patent Publication No. 75,444].

While the site for introducing a variation in the amino acid sequence is predetermined, the mutation per se need not be predetermined. For example, to optimize the performance of a mutation at a given site, random mutagenesis, such as linker scanning mutagenesis, may be conducted at a target codon or target region to create a large number of derivative which could then be expressed and screened for the optimal combination of desired activity. Alternatively, site-directed mutagenesis or other well-known technique may be employed to make mutations at predetermined sites in a DNA known sequence.

The technique of site-directed mutagenesis is well known in the art [see, e.g., Sambrook et al., supra, and McPherson (Ed.), Directed Mutagenesis: A Practical Approach, IRL Press, Oxford (1991)]. Site-directed mutagenesis allows the production of TANK2 functional derivatives through use of specific oligonucleotide sequences that encode the DNA sequence of the desired mutation. Site-directed mutagenesis methods and materials are commercially available, e.g., the QuikChange™ kit available from Stratagene (La Jolla, Calif.). One can selectively generate precise amino acid deletions, insertions, or substitutions using this method. Amino acid sequence deletions generally range from about 1 to 30 residues, more preferably 1 to 10 residues, and typically are contiguous. The most preferred deletions are those that are performed to generate catalytic fragments or DNA-binding fragments.

Mutations designed to increase the affinity of TANK2 may be guided by the introduction of the amino acid residues that are present at homologous positions in other poly(ADP-ribose) polymerase proteins. Similarly, such mutant TANK2 molecules may be prepared. that lack residues of a functional domain, e.g., the catalytic domain, to create a dominant negative protein.

It is difficult to predict a priori the exact effect any particular modification, e.g., substitution, deletion, insertion, etc., will have on the biological activity of TANK2. However, one skilled in the art will appreciate that the effect will be evaluated by routine screening assays. For example, a derivative typically is made by linker scanning site-directed mutagenesis of the DNA encoding the native TANK2 molecule. The derivative is then expressed in a recombinant host, and, optionally, purified from the cell culture, for example, by immunoaffinity chromatography. The activity of the cell lysate or the purified derivative is then screened in a suitable screening assay for the desired characteristic. For example, a change in the immunological character of the functional derivative, such as affinity for a given antibody, is measured by a competitive type immunoassay. Changes in other parameters of the expressed product may be measured by the appropriate assay.

Antibodies The present invention provides antibodies that bind with specificity to a TANK2 polypeptide. An “antibody” as used herein is defined broadly as a protein that characteristically immunoreacts with an epitope (antigenic determinant) that is characteristic of the TANK2 polypeptide. As used herein, an antibody is said to “immunoreact” with an antigen such as a polypeptide if the antibody specifically recognizes and binds an epitope that is characteristic of the antigen by way of one or more variable regions or one or more of the complementarity determining regions (CDRs) of the antibody.

An antibody that is immunoreactive with a given polypeptide may exhibit cross-reactivity to another polypeptide if the two polypeptides each comprise a common structural feature that defines the same characteristic epitope. In the case of related polypeptides, cross-reactivity can correlate to common structural features such as sequence identity, homology, or similarity found among the related polypeptides. Accordingly, families of polypeptides can often be identified by a cross-reactive antibody, i.e., an antibody that immunoreacts with some or all of the members of the polypeptide family sharing the common epitope. Thus, the invention encompasses antibodies that immunoreact with a particular member of the TANK2 family of polypeptides, e.g., a polypeptide comprising the amino acid sequence defined by SEQ ID NO:133 or SEQ ID NO:135. The invention further encompasses antibodies that immunoreact with some or all members of the TANK2 family of polypeptides. Screening assays to determine the binding specificity of an antibody are well known and routinely practiced in the art [see, e.g., Harlow et al. (Eds.), Antibodies: A Laboratory Manual, Ch. 6, Cold Spring Harbor Laboratory, Cold Spring Harbor N.Y. (1988)]. The immunoreactive specificity with which an antibody binds to a given polypeptide antigen is to be distinguished from interactions with other proteins, e.g., Staphylococcus aureus protein A or other antibodies in ELISA techniques, that are mediated through parts of the antibody other than the variable regions, in particular the constant regions of the antibody.

Antibodies include, for example, monoclonal antibodies, polyclonal antibodies, single chain antibodies (scFv antibodies), chimeric antibodies, multifunctional/multispecific (e.g., bifunctional or bispecific) antibodies, humanized antibodies, human antibodies, and CDR-grafted antibodies (including moieties that include CDR sequences that specifically immunoreact with a polypeptide of the invention). Antibodies according to the invention also include antibody fragments, so long as they exhibit the desired biological activity. “Antibody fragments” comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

Antibodies of the invention can be produced by any method known in the art. For example, polyclonal antibodies are isolated from mammals that have been immunized against the protein or a functional analog in accordance with methods known in the art. Briefly, polyclonal antibodies may be produced by injecting an immunogenic TANK2 polypeptide (immunogen) into a host mammal (e.g., rabbit, mouse, rat, or goat). Adjuvants may be employed to increase the immune response. Sera from the host mammal are extracted and screened to obtain polyclonal antibodies that are specific for (immunoreact with) the TANK2 polypeptide.

Monoclonal antibodies (also referred to herein as “mAbs”) are preferred. As used herein “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific (“monospecific”), being directed against a single antigenic site. Furthermore, in contrast to conventional (polyclonal) antibody preparations, which typically include different antibodies directed against different determinants (epitopes), each monoclonal antibody is directed against a single determinant on the antigen.

The modifier “monoclonal” indicates the character of the antibody as being obtained from a substantially homogeneous population of antibodies, and is not to be construed as requiring production of the antibody by any particular method. Monoclonal antibodies may be prepared using any suitable technique capable of yielding a continuous cell line producing a homogeneous antibody. Such methods include the immunological method [Kohler and Milstein, Nature 256:495-7 (1975); Campbell, “Monoclonal antibody technology, the production and characterization of rodent and human hybridomas” in Burdon et al. (Eds.), Laboratory Techniques in Biochemistry and Molecular Biology, Vol. 13, Elsevier Science Publishers, Amsterdam (1985)] or any similar method. Monoclonal antibodies may also be isolated from phage antibody libraries [Clackson et al., Nature 352:624-8 (1991); Marks et al., J Mol Biol 222:581-97 (1991)].

To illustrate, to produce monoclonal antibodies a host mammal is immunized by injection of an immunogenic TANK2 polypeptide, and then boosted. Spleens are collected from immunized mammals a few days after the final boost. Cell suspensions from the spleens are fused with a tumor cell line to create immortalized hybrid cell lines or “hybridomas.” Individual clones can be isolated by limiting dilution and then tested for the specificity of the antibodies they produce. Selected cells can then be grown, e.g., by the ascites method, to provide a continuous source of the desired homogeneous antibody.

Antibodies can be engineered using genetic techniques to produce chimeric antibodies including protein components from two or more species. For use in in vivo applications with a human subject, the antibody can be “humanized,” i.e., modified to contain an antigen binding region from one species, e.g., a rodent, with the bulk of the antibody replaced with sequences derived from human immunoglobulin. In one method, the non-human CDRs of one species e.g., a mouse or rabbit, are inserted into a framework sequence of another species, e.g., a human, or into a consensus framework sequence. Further changes can then be introduced into the antibody framework to modulate affinity or immunogenicity of the engineered antibody. Methods are also known for inducing expression of engineered antibodies in various cell types, such as mammalian and microbial cell types. Numerous techniques for preparing engineered antibodies are described in the art [e.g., Owens and Young, J Immunol Meth 168:149-65 (1994)].

Antibodies further include recombinant polyclonal or monoclonal Fab fragments [e.g., Huse et al., Science 246:1275-81 (1989)]. Alternatively, techniques described for the production of single chain antibodies [e.g., U.S. Pat. No. 4,946,778] can be adapted to produce TANK2-specific single chain antibodies (e.g., single chain Fv fragments; abbreviated “scFv”). Rapid, large-scale recombinant methods for generating antibodies may be employed, such as phage display or ribosome display methods, optionally followed by affinity maturation [see, e.g., Ouwehand et al., Vox Sang 74(Suppl 2):223-32 (1998); Rader et al., Proc Natl Acad Sci USA 95:8910-5 (1998); Dall'Acqua et al., Curr Opin Struct Biol 8:443-50 (1998)].

Fully human antibodies are especially preferred for therapeutic use in humans, but they are typically difficult to produce. For example, when the immunogen is a human self-antigen, a human will typically not produce any immune response to the antigen. Methods for making fully human antibodies have been developed and are known in the art. Accordingly, fully human antibodies can be produced by using an immunogenic TANK2 polypeptide to immunize an animal (e.g., mouse) that has been transgenically modified to express at least a significant fraction of the human repertoire of immunoglobulin genes [see, e.g., Bruggemann et al., Immunol Today 17:391-7 (1996)].

As noted herein, host cells of the invention are a valuable source of immunogen for development of antibodies specifically immunoreactive with TANK2. To be useful as an immunogen for the preparation of polyclonal or monoclonal antibodies, a TANK2 peptide fragment must contain sufficient amino acid residues to define an immunogenic epitope. If the fragment is too short to be immunogenic per se, it may be conjugated to a carrier molecule. Suitable carrier molecules include, for example, keyhole limpet hemocyanin (KLH) and bovine serum albumin (BSA). Conjugation may be carried out by methods known in the art. One such method is to combine a cysteine residue of the fragment with a cysteine residue on the carrier molecule.

Antibodies of the invention are useful for therapeutic methods (by modulating activity of TANK2), diagnostic methods (by detecting TANK2 in a sample), as well as purification of TANK2. The antibodies are particularly useful for detecting and/or quantitating TANK2 expression in cells, tissues, organs, and lysates and extracts thereof, as well as in fluids such as serum, plasma, cerebrospinal fluid, urine, sputum, peritoneal fluid, pleural fluid, or bronchoalveolar lavage fluid. Kits comprising an antibody of the invention for any of the purposes described herein are also contemplated. In general, a kit of the invention also includes a control antigen with which the antibody immunoreacts, and may further include other reagents, containers, and package inserts.

Further, the invention includes neutralizing antibodies, i.e., antibodies that significantly inhibit or impair a biological activity of the proteins or functional analogs of the invention. In particular, neutralizing antibodies inhibit or impair the poly(ADP-ribose) polymerase activity of TANK2. Neutralizing antibodies may be especially desirable for therapeutic and diagnostic applications.

Functional equivalents further include fragments of antibodies that have the same binding characteristics as, or that have binding characteristics comparable to, those of the whole antibody. Such fragments may contain one or both Fab fragments or the F(ab′)₂ fragment. Preferably, the antibody fragments contain all six complement determining regions (“CDRs”) of the whole antibody, although fragments containing fewer than all of such regions, such as three, four, or five CDRs, may also be functional. Fragments may be prepared by methods described in the art [e.g., Lamoyi et al., J Immunol Meth 56:235-43 (1983); Parham, J Immunol 131:2895-902 (1983)].

Moreover, specific binding proteins can be developed using isolated or recombinant TANK2 products, TANK2 variants, or cells expressing such products. Binding proteins are useful for purifying TANK2 products and detection or quantification of TANK2 products in fluid and tissue samples using known immunological procedures. Binding proteins are also manifestly useful in modulating (i.e., blocking, inhibiting, or stimulating) biological activities of TANK2 polypeptides, especially those activities involved in signal transduction. Thus, neutralizing antibodies that inhibit the activity of TANK2 polypeptides are provided. Anti-idiotypic antibodies specific for anti-TANK2 antibodies are also contemplated.

Detectable Polvnucleotide and Polypeptide Probes

The present invention further provides a method of detecting the presence of a TANK2-encoding polynucleotide or a TANK2 polypeptide in a sample. The method involves use of a labeled probe that recognizes the presence of a defined target in the sample. The probe may be an antibody that recognizes a TANK2 polypeptide, or an oligonucleotide that recognizes a polynucleotide encoding TANK2 polypeptide.

The probes of the invention can be detectably labeled in accordance with methods known in the art. In general, the probe can be modified by attachment of a detectable label (reporter) moiety to the probe, or a detectable probe can be manufactured with a detectable label moiety incorporated therein. The detectable label moiety can be any detectable moiety, many of which are known in the art, including radioactive atoms, electron dense atoms, enzymes, chromogens and colored compounds, fluorogens and fluorescent compounds, members of specific binding pairs, and the like.

Methods for labeling oligonucleotide probes have been described in the art [see, e.g., Leary et al., Proc Natl Acad Sci USA 80:4045-49 (1983); Renz and Kurz, Nucleic Acids Res 12:3435-44 (1984); Richardson and Gumport, Nucleic Acids Res 11:6167-84 (1983); Smith et al., Nucleic Acids Res 13:2399-412 (1985); Meinkoth and Wahl, Anal Biochem 138:267-84 (1984); and U.S. Pat. Nos. 4,711.955; 4,687,732; 5,241,060; 5,244,787; 5,328.824; 5,580,990; and 5,714,327].

Methods for labeling antibodies have been also been described [see. e.g., Hunter et al., Nature 144:495-6 (1962); David et al., Biochemistry 13:1014-21 (1974); and U.S. Pat. Nos. 3,940,475 and 3,645,090].

The label moiety may be radioactive. Some examples of useful radioactive labels include ³²P, ¹²⁵I, ¹³¹I, and ³H. Use of radioactive labels has been described [e.g., UK patent document 2,034,323 and U.S. Pat. Nos. 4,358,535 and 4,302,204].

Some examples of non-radioactive labels include enzymes, chromogens, atoms and molecules detectable by electron microscopy, and metal ions detectable by their magnetic properties.

Some useful enzymatic labels include enzymes that cause a detectable change in a substrate. Some useful enzymes (and their substrates) include, for example, horseradish peroxidase (pyrogallol and o-phenylenediamine), beta-galactosidase (fluorescein beta-D-galactopyranoside), and alkaline phosphatase (5-bromo-4-chloro-3-indolyl phosphate/nitro blue tetrazolium). The use of enzymatic labels has been described in the art [see, e.g., UK patent document 2,019,404, European patent document EP 63,879, and Rotman, Proc Nati Acad Sci USA 47:1981-91 (1961)].

Useful reporter moieties include, for example, fluorescent, phosphorescent, chemiluminescent, and bioluminescent molecules, as well as dyes. Some specific colored or fluorescent compounds useful in the present invention include, for example, fluoresceins, coumarins, rhodamines, Texas red, phycoerythrins, umbelliferones, Luminol®, and the like. Chromogens or fluorogens, i.e., molecules that can be modified (e.g., oxidized) to become colored or fluorescent or to change their color or emission spectra, are also capable of being incorporated into probes to act as reporter moieties under particular conditions.

The label moieties may be conjugated to the probe by methods that are well known in the art. The label moieties may be directly attached through a functional group on the probe. The probe either contains or can be caused to contain such a functional group. Some examples of suitable functional groups include, for example, amino, carboxyl, sulfhydryl, maleimide, isocyanate, isothiocyanate.

Alternatively, label moieties such as enzymes and chromogens may be conjugated to antibodies or nucleotides by means of coupling agents, such as dialdehydes, carbodiumides, dimaleimides, and the like.

The label moiety may also be conjugated to the probe by means of a ligand attached to the probe by a method described above and a receptor for that ligand attached to the label moiety. Any of the known ligand-receptor binding pair combinations is suitable. Some suitable ligand-receptor pairs include, for example, biotin-avidin or -streptavidin, and antibody-antigen. The biotin-streptavidin combination may be preferred.

Methods of Using Tankyrase2 Polynucleotides and Polypeptides

The scientific value of the information contributed through the disclosures of DNA and amino acid sequences of the present invention is manifest. As one series of examples, knowledge of the sequence of a cDNA for tank2 makes possible through use of Southern hybridization or polymerase chain reaction (PCR) the identification of genomic DNA sequences encoding TANK2 and TANK2 expression control regulatory sequences. DNA/DNA hybridization procedures carried out with DNA sequences of the invention under moderately to highly stringent conditions are also expected to allow the isolation of DNAs encoding allelic variants of TANK2. Similarly, non-human species genes encoding proteins homologous to TANK2 can also be identified by Southern and/or PCR analysis. As an alternative, complementation studies can be useful for identifying other human TANK2 products as well as non-human proteins, and DNAs encoding the proteins, sharing one or more biological properties of TANK-2. Oligonucleotides of the invention are also useful in hybridization assays to detect the capacity of cells to express TANK2. Polynucleotides of the invention may also be the basis for diagnostic methods useful for identifying a genetic alteration in the tank2 locus that underlies a disease state. For example, the differential expression or activity of TANK2-LONG and TANK2-SHORT may be capable of correlation with particular disease state(s), rendering one or both forms of TANK2 suitable as diagnostic markers or as therapeutic targets as described herein. Therefore, selective reagents, e.g., oligonucleotides that selectively hybridize to one form of tank2 or antibodies that selectively immunoreact with one form of TANK2, may be especially useful.

Oligonucleotides of the invention, as described herein, may be used in methods to amplify DNA for various purposes. “Amplification” according to the method of the invention refers to any molecular biology technique for detection of trace levels of a specific nucleic acid sequence by exponentially amplifying a template nucleic acid sequence. In particular, suitable amplification techniques include such techniques as the polymerase chain reaction (PCR), the ligase chain reaction (LCR) and variants thereof. PCR is known to be a highly sensitive technique, and is in wide use [see, e.g., Innis et al., PCR Protocols: A Guide to Methods and Applications, Academic Press, Inc., San Diego (1990); Dieffenbach and Dveksler, PCR Primer: A Laboratory Manual, Cold Spring Harbor Laboratory Press, Plainview N.Y. (1995); and U.S. Pat. Nos. 4,683,195; 4,800,195; and 4,965,188]. The more recently developed LCR technique is known to be highly specific, and is capable of detecting point mutations [see, e.g., Landegren et al., Science 241:1077-80 (1988) and Barany et al., PCR Methods and Applications 1:5-16 (1991)]. An LCR kit is available from Stratagene. In certain circumstances, it is desirable to couple the PCR and LCR techniques to improve precision of detection. Other amplification techniques may be employed in accordance to the invention.

Oligonucleotide amplification primers are often provided as matched pairs of single-stranded oligonucleotides; one with sense orientation (5′→3′) and one with antisense (3′←5′) orientation. Such specific primer pairs can be employed under optimized conditions for identification of a specific gene or condition. Alternatively, the same primer pair, nested sets of oligomers, or even a degenerate pool of oligomers, may be employed under less stringent conditions for detection and/or quantitation of closely related DNA or RNA sequences.

Such oligonucleotides can be used in various methods known in the art to extend the specified nucleotide sequences. These methods permit use of a known sequence to determine unknown adjacent sequence, thereby enabling detection and determination of upstream sequences such as promoters and regulatory elements.

For example, restriction-site polymerase chain reaction is a direct method that uses universal primers to retrieve unknown sequence adjacent to a known locus [see. e.g., Gobinda et al., PCR Methods Applic 2:318-22 (1993)]. In this method, genomic DNA is first amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region [Triglia et al., Nucleic Acids Res 16:8186 (1988)]. The primers may be designed using Oligo 4.0 (National Biosciences, Inc., Plymouth, Minn.), or another appropriate program, to be 22-30 nucleotides in length, to have a GC content of 50% or more, and to anneal to the target sequence at temperatures about 68°-72° C. This method uses several restriction enzymes to generate a suitable fragment in the known region of a gene. The fragment is then circularized by intermolecular ligation and used as a PCR template.

Capture PCR is a method for PCR amplification of DNA fragments adjacent to a known sequence in human and yeast artificial chromosome (YAC) DNA [Lagerstrom et al., PCR Methods Applic 1:111-9 (1991)]. Capture PCR also requires multiple restriction enzyme digestions and ligations to place an engineered double-stranded sequence into an unknown portion of the DNA molecule before PCR. Walking PCR is a method for targeted gene walking that permits retrieval of unknown sequence [Parker et al., Nucleic Acids Res 19:3055-60 (1991)]. The PromoterFinder™ kit (Clontech, Palo Alto, Calif.) uses PCR, nested primers, and special libraries to “walk in” genomic DNA. This process avoids the need to screen libraries and is useful in finding intron/exon junctions.

Such methods can be used to explore genomic libraries to extend 5′ sequence and to obtain endogenous tank2 genomic sequence, including elements such as promoters, introns, operators, enhancers, repressors, and the like. Preferred libraries for screening for full-length cDNAs are ones that have been size-selected to include larger cDNAs. In addition, randomly primed libraries are preferred in that they will contain more sequences that contain the 5′ and upstream regions of genes.

The oligonucleotide probes may also be used for mapping the endogenous genomic sequence. The sequence may be mapped to a particular chromosome or to a specific region of the chromosome using well known techniques. These include in situ hybridization to chromosomal spreads [Venna et al., Human Chromosomes: A Manual of Basic Technique, Pergamon Press, New York N.Y. (1988)], flow-sorted chromosomal preparations, or artificial chromosome constructions such as YACs, bacterial artificial chromosomes (BACs), bacterial P1 constructions, or single chromosome cDNA libraries.

Hybridization of chromosomal preparations and physical mapping techniques such as linkage analysis using established chromosomal markers are invaluable in extending genetic maps. Examples of genetic maps can be found in the art [e.g., Hodgkin et al., Science 270:410-4 (1995) and Murray et al., Science 265:2049-54 (1994)]. Often the placement of a gene on the chromosome of another mammalian species may reveal associated markers even if the number or arm of a particular human chromosome is not known. Such sequences can be assigned to particular structural features of chromosomes by physical mapping. This provides valuable information to investigators searching for disease genes using positional cloning or other gene discovery techniques. Once a disease or syndrome has been crudely localized by genetic linkage to a particular genomic region, any sequences mapping to that area may represent associated or regulatory genes for further investigation. See, e.g., Gatti et al., Nature 336:577-80 (1988). The polynucleotides of the invention may also be used to detect differences in the chromosomal location due to translocation, inversion, etc., between normal, carrier, or affected individuals. Other types of genetic maps can also be developed, e.g., physical maps of the genome based on sequence-tagged sites (STS) [see, e.g., Hudson et al., Science 270:1945-54 (1995)].

The DNA sequence information provided by the present invention also makes possible the development, e.g., through homologous recombination or “knock-out” strategies [Capecchi, Science 244:1288-92 (1989)], of animals that fail to express functional TANK2 or that express a Xariant of TANK2. Such animals are useful as models for studying the in vivo activities of TANK-2 and modulators thereof.

As described herein, the invention provides antisense nucleic acid sequences that recognize and hybridize to polynucleotides encoding TANK2. Modifications of gene expression can be obtained by designing antisense sequences to the control regions of the tank2 gene, such as the promoters, enhancers, and introns. Oligonucleotides derived from the transcription initiation site, e.g., between −10 and +10 regions of the leader sequence, are preferred. Antisense RNA and DNA molecules may also be designed to block translation of mRNA by preventing the transcript from binding to ribosomes. The worker of ordinary skill will appreciate that antisense molecules of the invention include those that specifically recognize and hybridize to tank2 DNA (as determined by sequence comparison of tank2 DNA to DNA encoding other known molecules). The antisense molecules of the invention also include those that recognize and hybridize to DNA encoding other members of the TANK2 family of proteins. Antisense polynucleotides that hybridize to multiple DNAs encoding other members of the TANK2 family of proteins are also identifiable through sequence comparison to identify characteristic or signature sequences for the family of TANK2 proteins. Accordingly, such antisense molecules preferably have at least 95%, more preferably at least 98%, and still more preferably at least 99% identity to the target tank2 sequence.

Antisense polynucleotides are particularly relevant to regulating expression of TANK2 by those cells expressing tank2 mRNA. Antisense polynucleotides (preferably 10 to 20 bp oligonucleotides) capable of specifically binding to tank2 expression control sequences or tank2 RNA are introduced into cells, e.g., by a viral vector or a colloidal dispersion system such as a liposome. The antisense oligonucleotide binds to the tank2 target nucleotide sequence in the cell and prevents transcription or translation of the target sequence. Phosphorothioate and methylphosphonate antisense oligonucleotides are specifically contemplated for therapeutic use under the invention. The antisense oligonucleotides may be further modified by poly-L-lysine, transferrin polylysine, or cholesterol moieties at their 5 ends [for a recent review of antisense technology, see Delihas et al., Nat Biotechilol 15:751-3 (1997)].

The invention further comprises methods to modulate TANK2 expression by means of ribozyme technology [for a review, see Gibson and Shillitoe, Mol Biotechnol 7:125-37 (1997)]. Ribozyme technology can be used to inhibit translation of tank2 mRNA in a sequence specific manner through (i) the hybridization of a complementary RNA to a target mRNA and (ii) cleavage of the hybridized mRNA through endonuclease activity inherent to the complementary RNA. Ribozymes can be identified by empirical methods such as using complementary oligonucleotides in ribonuclease protection assays, but more preferably are specifically designed based on scanning the target molecule for accessible ribozyme cleavage sites [Bramlage et al., Trends Biotechnol 16:434-8 (1998)]. Delivery of ribozymes to target cells can be accomplished using either exogenous or endogenous delivery techniques well known and practiced in the art. Exogenous can include use of targeting liposomes or direct local injection. Endogenous methods include use of viral vectors and non-viral plasmids.

Ribozymes can specifically modulate expression of TANK2 when designed to be complementary to regions unique to a polynucleotide encoding TANK2. “Specifically modulate,” therefore is intended to mean that ribozymes of the invention recognize only a polynucleotide encoding TANK2. Similarly, ribozymes can be designed to modulate expression of all or some of the TANK2 family of proteins. Ribozymes of this type are designed to recognize nucleotide sequences conserved all or some of the polynucleotides encoding the TANK2 family members.

The invention further embraces methods to modulate transcription of tank2 through use of oligonucleotide-directed triple helix formation (also known as Hogeboom base-pairing methodology) [for a review, see Lavrovsky et al., Biochem Mol Med 62:11-22 (1997)]. Triple helix formation is accomplished using sequence-specific oligonucleotides that hybridize to double stranded DNA in the major groove as defined in the Watson-Crick model. This triple helix hybridization compromises the ability of the original double helix to open sufficiently for the binding of polymerases, transcription factors, or regulatory molecules. Preferred target sequences for hybridization include promoter and enhancer regions to permit transcriptional regulation of TANK2 expression. Oligonucleotides that are capable of triple helix formation can alternatively be coupled to DNA damaging agents, which can then be used for site-specific covalent modification of target DNA sequences [see Lavrovsky et al., supra].

Both antisense RNA and DNA molecules and ribozymes of the invention may be prepared by any method known in the art for the synthesis of RNA molecules. These include techniques for chemically synthesizing oligonucleotides such as solid-phase phosphoramidite chemical synthesis. Alternatively, RNA molecules may be generated by in vitro or in vivo transcription of DNA sequences encoding the antisense RNA molecule. Such DNA sequences may be incorporated into a variety of vectors with suitable RNA polymerase promoters such as T7 or SP6. Alternatively, antisense cDNA constructs that synthesize antisense RNA constitutively or inducibly can be introduced into cell lines, cells, or tissues.

Mutations in a gene that result in loss of normal function of the gene product may underlie TANK2-related disease states. The invention comprehends gene therapy to restore TANK2 activity as indicated in treating those disease states characterized by a deficiency or absence of poly(ADP-ribose) polymerase activity associated with the TANK2 enzyme. Delivery of functional tank2 gene to appropriate cells is effected ex vivo, in situ, or in vivo by use of vectors, and more particularly viral vectors (e.g., adenovirus, adeno-associated virus, or retrovirus), or ex vivo by use of physical DNA transfer methods (e.g., liposomes or chemical treatments) [see, e.g., Anderson, Nature 392(6679 Suppl):25-30 (1998)]. Alternatively, it is contemplated that in other disease states, preventing the expression or inhibiting the activity of TANK2 will be useful in treating those disease states. Antisense therapy or gene therapy can be applied to negatively regulate the expression of TANK2.

The DNA and amino acid sequence information provided by the present invention also makes possible the systematic analysis of the structure and function of TANK2 proteins. DNA and amino acid sequence information for TANK2 also permits identification of molecules with which a TANK2 polypeptide will interact. Agents that modulate (i.e.. increase, decrease, or block) TANK2 activity may be identified by incubating a putative modulator with TANK2 and determining the effect of the putative modulator on TANK2 activity. The selectivity of a compound that modulates the activity of the TANK2 polypeptide can be evaluated by comparing its activity on the TANK2 to its activity on other proteins.

Numerous methods are amenable to modification by including TANK2 polypeptides or tank2 polynucleotides of the invention, including cell based methods such as dihybrid and trihybrid screens to detect binding partners and split hybrid screens to detect compounds that disrupt complexing of binding partners. Other methods include in vitro methods, such as assays in which a TANK2 polypeptide, tank2 polynucleotide, or a binding partner thereof is immobilized, as well as solution assays, are contemplated under the invention. These methods are exemplified by a general approach that includes the steps of contacting a TANK2 polypeptide with a putative binding partner compound, detecting or measuring binding of the TANK2 polypeptide with the compound, and optionally isolating and/or identifying the binding partner compound.

Cell-based assays include methods of screening genomic DNA or cDNA libraries to identify binding partners of TANK2 polypeptides. Exemplary methods include the dihybrid or two-hybrid screen [Fields and Song, Nature 340:245-6 (1989); Fields, Methods: A Companion to Methods in Enzymology 5:116-24 (1993)] which can be used identify DNAs encoding binding partners. Modifications and variations of the dihybrid assay are described [Colas and Brent, Trends Biotechnol 16:355-63 (1998)]. Trihybrid screens can also be employed [Fuller et al., Biotechniques 25:85-8, 90-2 (1998)].

Cell-based methods of the invention may be used to identify components in biological pathways that are mediated by TANK2 biological activity. In one aspect, the method is carried out in a host cell containing a soluble TANK2 polypeptide and a soluble form of its binding partner and wherein decreased of increased binding is quantitated through measurement of a binding-dependent phenotypic change in the host cell that is associated with a change in expression of a reporter gene product.

Alternatively, cell-based assays to identify inhibitors of TANK2 polypeptide interaction with a known binding partner may be based on methods such as the split hybrid assay [PCT patent publication WO 98/13502] and variations thereof [PCT patent publication WO 95/20652].

In vitro methods can comprise the steps of (a) contacting an immobilized TANK2 polypeptide with a candidate binding partner compound, and (b) detecting binding of the candidate compound to the TANK2 polypeptide. In an alternative embodiment, the candidate binding partner compound is immobilized and binding of the TANK2 polypeptide is detected. Immobilization may be accomplished using any of the methods well known in the art, including bonding to a support, beads, or a chromatographic resin, as well as high affinity interactions such as antibody binding or use of an avidin:biotin type system. Detection of binding of the ligands can be accomplished, for example, by (i) using a detectable (e.g., radioactive or fluorescent) label on the ligand that is not immobilized, (ii) using an antibody immunospecific for the non-immobilized ligand, (iii) using a label on the non-immobilized ligand that promotes excitation of a fluorescent support to which the immobilized ligand is bound, as well as other techniques routinely practiced in the art.

In solution assays, methods of the invention comprise the steps of (a) contacting a TANK2 polypeptide with one or more candidate binding partner compounds, and (b) identifying the compounds that bind to the TANK2 polypeptide. Identification of the compounds that bind TANK2 can be achieved by isolating the TANK2:binding partner complex, and separating the TANK2 polypeptide from the binding partner compound. An additional step of characterizing the physical, biological, or biochemical properties of the binding partner compound is also comprehended under the invention. In one approach the TANK2:binding partner complex is isolated using a second binding partner compound (e.g., an antibody or other protein) that interacts with either of the principal ligands in the complex.

Selective modulators may include, for example, antibodies and other proteins or peptides that selectively or specifically bind to a TANK2 polypeptide or a TANK2-encoding polynucleotide, oligonucleotides that selectively or specifically bind to TANK2 polypeptides or TANK2-encoding polynucleotides, and other non-peptide compounds (e.g., isolated or synthetic organic molecules) that selectively or specifically react with TANK2 polypeptides or TANK2-encoding polynucleotides. Modulators also include compounds as described above but which interact with a specific binding partner of TANK2 polypeptides. Mutant formns of TANK2, such as those that affect the biological activity or cellular location of the wild-type TANK2, are also contemplated under the invention. Presently preferred targets for the development of selective modulators include, for example:

(1) cytoplasmic or transmembrane regions of TANK2 polypeptides that contact other proteins and/or localize TANK2 within a cell, e.g., to telomeres;

(2) extracellular regions of TANK2 polypeptides that bind specific binding partners;

(3) regions of the TANK2 polypeptides that bind substrate, i.e., ADP-ribose;

(4) allosteric regulatory sites of the TANK2 polypeptides;

(5) regions of the TANK2 polypeptides that mediate multimerization;

(6) regions of TANK2 or other proteins (e.g., TRF1 or TRF2) that act as acceptors ADP-ribosylation.

Still other selective modulators include those that recognize particular regulatory or TANK2-encoding nucleotide sequences. Selective and specific modulators of TANK2 activity may be therapeutically useful in treatment of a wide range of diseases and physiological conditions in which aberrant TANK2 activity is involved.

A TANK2-encoding polynucleotide sequence may be used for the diagnosis of diseases resulting from or associated with TANK2 expression or activity. For example, polynucleotide sequences encoding a TANK2 polypeptide (e.g., TANK2-LONG or TANK2-SHORT) may be used in hybridization or PCR assays of biological samples, e.g., samples or extracts of fluids or tissues from biopsies or autopsies, to detect abnormalities in TANK2 expression. Such qualitative or quantitative methods may include Southern or northern analysis, dot blot, or other membrane-based technologies; PCR technologies; dipstick, pin or chip technologies; and ELISA or other multiple-sample format technologies. These types of techniques are well known in the art and have been employed in commercially available diagnostic kits.

Such assays may be tailored to evaluate the efficacy of a particular therapeutic treatment regimen and may be used in animal studies, in clinical trials, or in monitoring the treatment of an individual patient. To provide a basis for the diagnosis of disease, a normal or standard profile for TANK2 expression must be established. This is accomplished by combining a biological sample taken from a normal subject with a tank2 polynucleotide, under conditions suitable for hybridization or amplification. Standard hybridization may be quantified by comparing the values obtained for normal subjects with a dilution series of positive controls run in the same experiment where a known amount of a purified tank2 polynucleotide is used. Standard values obtained from normal samples may be compared with values obtained from samples from subjects potentially affected by a disorder or disease related to TANK2 expression. Deviation between standard and subject values establishes the presence of the disease state. If disease is established, an existing therapeutic agent is administered, and treatment profile or values may be generated. The assay may be repeated on a regular basis to evaluate whether the values progress toward or return to the normal or standard pattern. Successive treatment profiles may be used to show the efficacy of treatment over a period of several days or several months.

Anti-TANK2 antibodies are useful for the diagnosis of conditions, disorders, or diseases characterized by or associated with abnormal expression of a TANK2 polypeptide. Diagnostic assays for TANK2 polypeptides include methods that employ a labeled antibody to detect a TANK2 polypeptide in a biological sample such as a body fluid, cells, tissues, sections, or extracts of such materials. The polypeptides and antibodies of the present invention may be used with or without modification. Preferably, the polypeptide or the antibody will be labeled by linking them, either covalently or non-covalently, with a detectable label moiety as described herein.

Antibody-based methods for detecting the presence of TANK2 polypeptides in biological samples are enabled by virtue of the present invention, including assays for differential detection of TANK2-LONG versus TANK2-SHORT. Assays for detecting the presence of proteins with antibodies have been previously described, and follow known formats, such as enzyme-linked immunosorbent assay (ELISA), radioimmunoassay (RIA), and fluorescence activated cell sorting (FACS) and flow cytometry, western blots, sandwich assays, and the like. These formats are normally based on incubating an antibody with a sample suspected of containing the TANK2 protein and detecting the presence of a complex between the antibody and the protein. The antibody is labeled either before, during, or after the incubation step. The specific concentrations of antibodies, the temperature and time of incubation, as well as other such assay conditions, can be varied, depending upon various factors including the concentration of antigen in the sample, the nature of the sample, etc. Those skilled in the art will be able to determine operative and optimal assay conditions for each determination by employing routine experimentation [see, e.g., Hampton et al., Serological Methods: A Laboratory Manual, APS Press, St Paul, Minn. (1990)].

To provide a basis for the quantitation of TANK2 protein in a sample or for the diagnosis of disease, normal or standard values of TANK2 polypeptide expression must be established. This is accomplished by combining body fluids or cell extracts taken from a normal sample or from normal subjects, either animal or human, with antibody to a TANK2 polypeptide. The amount of standard complex formation may be quantified by comparing it with a dilution series of positive controls where a known amount of antibody is combined with known concentrations of a purified TANK2 polypeptide. Then, standard values obtained from normal samples may be compared with values obtained from samples from test sample, e.g., subjects potentially affected by a disorder or disease related to a TANK2 expression. Deviation between standard and test values establishes the presence of the disease state.

Methods for Identifying Modulators of Tankyrase2 Activity

The TANK2 protein, as well as fragments thereof possessing biological activity can be used for screening putative modulator compounds in any of a variety of drug screening techniques. The term “modulator” as used herein refers to a compound that acts as an agonist or as an antagonist of TANK2 activity. Modulators according to the invention include allosteric modulators of activity as well as inhibitors of activity. An “agonist” of TANK2 is a compound that enhances or increases the ability of TANK-2 to carry out any of its biological functions. An example of such an agonist is an agent that increases the ability of TANK2 to bind to damaged DNA or to polymerize ADP-ribose. An “antagonist” of TANK2 is a compound that diminishes or abolishes the ability of TANK2 to carry out any of its biological functions. An example of such antagonists is an anti-TANK2 antibody.

Accordingly, the invention provides a method for screening a plurality of test compounds for specific binding affinity with a TANK2 polypeptide, comprising providing a plurality of test compounds; combining a TANK2 polypeptide with each of the plurality of test compounds for a time sufficient to allow binding under suitable conditions; and detecting binding of the TANK2 polypeptide to each of the plurality of test compounds, thereby identifying those test compounds that specifically bind the TANK2 polypeptide.

The present invention also provides a method of identifying a modulator of a biological activity of a TANK2 polypeptide, comprising the steps of a) contacting the compound with a TANK2 polypeptide, b) incubating the mixture of step a) with a substrate under conditions suitable for the biological activity, c) measuring the amount of the biological activity; and d) comparing the amount of biological activity of step c) with the amount of biological activity obtained with the TANK2 polypeptide, incubated without the compound, thereby determnining whether the compound stimulates or inhibits the biological activity. In one embodiment of the method, the TANK2 polypeptide is a fragment from the non-catalytic region of the TANK2 and provides a method to identify allosteric modulators of TANK2. In another embodiment, the TANK2 polypeptide is a fragment from the catalytic region of TANK2 and provides a method to identify inhibitors of the biological activity. TANK2-LONG and TANK2-SHORT polypeptides or specific fragments thereof may be employed.

Accordingly, the polypeptide employed in such methods may be free in solution, affixed to a solid support, displayed on a cell surface, or located intracellularly. The modulation of activity or the formation of binding complexes between the TANK2 polypeptide and the agent being tested may be measured. TANK2 polypeptides are amenable to biochemical or cell-based high throughput screening (HTS) assays according to methods known and practiced in the art, including melanophore assay systems to investigate receptor-ligand interactions, yeast-based assay systems, and mammalian cell expression systems [for a review. see Jayawickreme and Kost, CuWr Opin Biotechnol 8:629-34 (1997)]. Automated and miniaturized HTS assays are also comprehended [e.g., Houston and Banks, Curr Opin Biotechnol 8:734-40 (1997)].

Such HTS assays are used to screen libraries of compounds to identify particular compounds that exhibit a desired property. Any library of compounds may be used, including chemical libraries, natural product libraries, combinatorial libraries comprising random or designed oligopeptides, oligonucleotides, or other organic compounds.

Chemical libraries may contain known compounds, proprietary structural analogs of known compounds, or compounds that are identified from natural product screening.

Natural product libraries are collections of materials isolated from naturals sources, typically, microorganisms, animals, plants, or marine organisms. Natural products are isolated from their sources by fermentation of microorganisms followed by isolation and extraction of the fermentation broths or by direct extraction from the microorganisms or tissues (plants or animal) themselves. Natural product libraries include polyketides, non-ribosomal peptides, and variants (including non-naturally occurring variants) thereof [for a review, see Cane et al., Science 282:63-8 (1998)].

Combinatorial libraries are composed of large numbers of related compounds, such as peptides, oligonucleotides, or other organic compounds as a mixture. Such compounds are relatively straightforward to design and prepare by traditional automated synthesis protocols, PCR, cloning or proprietary synthetic methods. Of particular interest are peptide and oligonucleotide combinatorial libraries.

Still other libraries of interest include peptide, protein, peptidomimetic, multiparallel synthetic collection, recombinatorial, and polypeptide libraries [for a review of combinatorial chemistry and libraries created thereby, see Myers, Curr Opin Biotechnol 8:701-7 (1997)].

Once compounds have been identified that show activity as modulators of TANK2 function, a program of optimization can be undertaken in an effort to improve the potency and or selectivity of the activity. This analysis of structure-activity relationships (SAR) typically involves of iterative series of selective modifications of compound structures and their correlation to biochemical or biological activity. Families of related compounds can be designed that all exhibit the desired activity, with certain members of the family potentially qualifying as therapeutic candidates.

The invention also encompasses the use of competitive drug screening assays in which neutralizing antibodies capable of binding a TANK2 polypeptide specifically compete with a test compound for binding to the TANK2 polypeptide. In this manner, the antibodies can be used to detect the presence of any compound, e.g., another peptide that shares one or more antigenic determinants with the TANK2 polypeptide.

Therapeutic Uses of TANK2-Encoding Polynucleotides and TANK2 Polypeptides

The invention provides a method for inhibiting the expression or activity of TANK2 therapeutically or prophylactically in a human or other animal. The method comprises administering a TANK2 antagonist in an amount effective for inhibiting TANK2 expression or activity. The invention thus provides a method for treating tissue damage resulting from cell damage or death due to necrosis or apoptosis, comprising administering to the animal an effective amount of a compound that inhibits TANK2 activity. This method may be employed in treating animals that are or may be subject to any disorder whose symptoms or pathology is mediated by TANK2 expression or activity. Antagonists having specificity for TANK2-LONG or TANK2-SHORT may have particular utility in diseases whose pathology or symptoms are mediated by a specific form of TANK2.

The method may further involve administering an antagonist of another poly(ADP-ribose) polymerase activity, such as activity associated with the enzymes PARP, tankyrase 1, and the like. Exemplary PARP antagonists suitable for use in this embodiment include, for example, the compounds described by Banasik et al. [J Biol Chem 267:1569-75 (1992)]. Other exemplary compounds include those described in PCT patent publications WO 99/11623 and WO 99/11649. Alternatively, the TANK2 inhibitory method may entail use of a compound that antagonizes both TANK2 and another enzyme having poly(ADP-ribose) polymerase activity.

“Treating” as used herein refers to preventing a disorder from occurring in an animal that may be predisposed to the disorder, but has not yet been diagnosed as having it; inhibiting the disorder, i.e., arresting its development; relieving the disorder, i.e., causing its regression, or ameliorating the disorder, i.e., reducing the severity of symptoms associated with the disorder. “Disorder” is intended to encompass medical disorders, diseases, conditions, syndromes, and the like, without limitation.

The methods of the invention embrace various modes of treating an animal in which TANK2 is expressed, and in which TANK2-mediated disorders may be treated. Animals treatable according to the invention include mammals (including humans) and non-mammalian animals, e.g., birds, fish, reptiles, and amphibians. Among the non-human mammals that may be treated are companion animals (pets) including dogs and cats; farm animals including cattle, horses; sheep, pigs, and goats; laboratory animals including rats, mice, rabbits, guinea pigs, and primates. The method is most preferably employed in the treatment of TANK2-mediated disorders in humans.

In particular, the method of the invention may be employed to treat animals therapeutically or prophylactically who are or may subject to a disorder associated with excessive or undesirable telomerase activity. One aspect of the present invention derives from the ability of TANK2 and its functional derivatives to interact with damaged DNA and to modulate the activity of telomere repeat binding factors (e.g., TRF1 and TRF2).

Excessive telomerase activity in cells has been shown to correlate with induction of apparently unlimited capacity of the cells to replicate. In addition, evidence exists that telomerase activity is higher in tumor tissue than most normal tissues suggesting that increased telomerase activity may be essential for tumor growth. Accordingly, the invention also provides to a method of inhibiting oncogenic transformation or inhibiting neoplastic tissue growth, e.g., cancer, in an animal, comprising administering to the animal an effective amount of a compound that inhibits TANK2 activity. In this embodiment, the method may further comprise adjuvant administration of a chemotherapeutic or anti-cancer drug and/or radiation therapy.

Tumors or neoplasms include new growths of tissue in which the multiplication of cells is uncontrolled and progressive. Some such growths are benign, but others are termed “malignant,” leading to death of the organism. Malignant neoplasms or “cancers” are distinguished from benign growths in that, in addition to exhibiting aggressive cellular proliferation, cancers invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Neoplasms treatable by the present invention include solid tumors, i.e., carcinomas and sarcomas. Carcinomas include those malignant neoplasms derived from epithelial cells which tend to infiltrate (invade) the surrounding tissues and give rise to metastases. Adenocarcinomas are carcinomas derived from glandular tissue or in which the tumor cells form recognizable glandular structures. Another broad category of cancers includes sarcomas, which are tumors whose cells are embedded in a fibrillar or homogeneous substance like embryonic connective tissue. The invention also enables treatment of cancers of the myeloid or lymphoid systems, including leukemias, lymphomas and other cancers that typically do not present as a tumor mass, but are distributed in the vascular or lymphoreticular systems.

The type of cancer or tumor cells amenable to treatment according to the invention include, for example, ACTH-producing tumor, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and non-small cell), malignant peritoneal effusion, malignant pleural effusion, melanoma, mesothelioma, multiple myeloma, neuroblastoma. glioma, non-Hodgkin's lymphomlia, osteosarcoma, ovarian cancer, ovarian (germ cell) cancer, pancreatic cancer, penile cancer, prostate cancer. retinoblastoma, skin cancer, soft tissue sarcoma, squamous cell carcinomas. stomach cancer, testicular cancer, thyroid cancer, trophoblastic neoplasms, uterine cancer, vaginal cancer, cancer of the vulva, and Wilm's tumor.

As noted above, regulation of telomere structure appears to be associated with aging. Drugs that modulate the regulation of telomere structure can be expected to have utility in treatment of age-related syndromes or in cases of genetically determined premature aging and premature senility syndromes e.g., progeria (Hutchinson-Gilford progeria syndrome), Werner's syndrome, and other such disorders. Accordingly, the invention provides a method of enhancing the activity of TANK2 in animals suffering from such syndromes. The method may be expected to decrease TRF binding to the telomeres, which in turn promotes increased telomerase activity.

Shortening of telomeres beyond a critical length results in the induction of senescence in many cell types. As telomerase activity is frequently required for maintenance of telomere length, and since TANK2 inhibition may diminish telomerase function, the invention provides for treatment of non-neoplastic proliferative disorders in which TANK2 antagonists may be useful to induce shortened telomeres and cellular senescence. Proliferative disorders include, but are not limited to, andrestenosis, diabetic retinopathy, mesangial proliferative disorder, proliferative glomerulonephritis, polycythemia, myelofibrosis, post-transplantation lymphoproliferative disorder, endometriosis, craniosynostosis, immunoproliferative small intestinal disease, thymic lymphoproliferative disease, myelodysplastic disorders, myeloproliferative disorders, von Willebrand's disease, and proliferative nephritis.

In addition, TANK2 inhibitors may be useful in any inflammatory disorder, including autoimmune disorders, in which proliferation of lymphocytes plays a role. “Inflammatory disorder” as used herein can refer to any disease, disorder, or syndrome in which an excessive or unregulated inflammatory response leads to excessive inflammatory symptoms, host tissue damage, or loss of tissue function. “Inflammatory disorders” can also refer to pathological states mediated bv influx of leukocytes and or neutrophil chemotaxis.

“Inflammation” as used herein refers to a localized, protective response elicited by injury or destruction of tissues, which serves to destroy, dilute or wall off (sequester) both the injurious agent and the injured tissue. Inflammation is notably associated with influx of leukocytes and or neutrophil chemotaxis. Inflammation may result from infection with pathogenic organisms and viruses and from noninfectious means such as trauma or reperfusion following myocardial infarction or stroke, immune response to foreign antigen, and autoimmune responses. Inflammatory disorders amenable to the invention encompass disorders associated with reactions of the specific defense system as well as with reactions of the non-specific defense system.

Accordingly, the present invention enables methods of treating such inflammatory disorders as arthritic diseases, such as rheumatoid arthritis, osteoarthritis, gouty arthritis, spondylitis; Behcet disease; sepsis, septic shock, endotoxic shock, gram negative sepsis, gram positive sepsis, and toxic shock syndrome; multiple organ injury syndrome secondary to septicemia, trauma, or hemorrhage; ophthalmic disorders such as allergic conjunctivitis, vernal conjunctivitis, uveitis, and thyroid-associated ophthalmopathy; eosinophilic granuloma; pulmonary or respiratory disorders such as asthma, chronic bronchitis, allergic rhinitis, ARDS, chronic pulmonary inflammatory disease (e.g., chronic obstructive pulmonary disease), silicosis, pulmonary sarcoidosis, pleurisy, alveolitis, vasculitis, pneumonia, bronchiectasis, and pulmonary oxygen toxicity; reperfusion injury of the myocardium, brain, or extremities; fibrosis such as cystic fibrosis; keloid formation or scar tissue formation; atherosclerosis; autoimmune diseases such as systemic lupus erythematosus (SLE), autoimmune thyroiditis, multiple sclerosis, some forms of diabetes, and Reynaud's syndrome; and transplant rejection disorders such as GVHD and allograft rejection; chronic glomerulonephritis; inflammatory bowel diseases such as Crohn's disease, ulcerative colitis and necrotizing enterocolitis; inflammatory dermatoses such as contact dermatitis, atopic dermatitis, psoriasis, or urticaria; fever and myalgias due to infection; central or peripheral nervous system inflammatory disorders such as meningitis, encephalitis, and brain or spinal cord injury due to minor trauma; Sjögren's syndrome; diseases involving leukocyte diapedesis; alcoholic hepatitis; bacterial pneumonia; antigen-antibody complex mediated diseases; hypovolemic shock; Type I diabetes mellitus; acute and delayed hypersensitivity; disease states due to leukocyte dyscrasia and metastasis; thermal injury; granulocyte transfusion associated syndromes; and cytokine-induced toxicity.

The tank2 polynucleotides provided by the invention also enable therapeutic applications of these polynucleotides in treating the diseases and disorders described herein whose etiology involves TANK2 expression or activity. For example, a tank2 antisense molecule may provide the basis for treatment of various abnormal conditions related to excessive or undesirable levels of poly(ADP-ribose) polymerase activity. Alternatively, polynucleotide sequences encoding TANK2 may provide the basis for the treatment of various abnormal conditions related to deficiency of poly(ADP-ribose) polymerase activity. Polynucleotides having specificity for one or both of tank2-long and tank2-short may have particular utility in certain diseases.

Expression vectors derived from retroviruses, adenovirus, herpes, or vaccinia viruses, or from various bacterial plasmids, may be used for delivery of recombinant tank2 sense or antisense molecules to the targeted cell population. Methods that are well known to those skilled in the art can be used to construct recombinant vectors containing tank2. See, for example, the techniques described in Sambrook et al., supra, and Ausubel et al., supra. Alternatively, recombinant tank2 can be delivered to target cells in liposomes.

The cDNA sequence, and/or its regulatory elements, enables researchers to use a tank2 polynucleotide as a tool in sense [Youssoufian and Lodish, Mol Cell Biol 13:98-104 (1993)] or antisense [Eguchi et al., Annu Rev Biochem 60:631-52 (1991)] investigations of gene function. Oligonucleotides, designed from the cDNA or control sequences obtained from the genomic DNA, can be used in vitro or in vivo to inhibit expression. Such technology is now well known in the art, and sense or antisense oligonucleotides or larger fragments can be designed from various locations along the coding or control regions. Again, tank2-long- or tank2-short-specific sequences may have distinct utilities depending on which form of tank2 is of interest.

Additionally, TANK-2 expression can be modulated by transfecting a cell or tissue with expression vectors that express high levels of a tank2 poly`nucleotide fragment in conditions where it would be preferable to block a biological activity of TANK2. Such constructs can flood cells with untranslatable sense or antisense sequences. Even in the absence of integration into the DNA, such vectors may continue to transcribe RNA molecules until all copies of the vector are disabled by endogenous nucleases. Such transient expression may be accomplished using a non-replicating vector or a vector incorporating appropriate replication elements.

Methods for introducing vectors into cells or tissue include those methods discussed herein. In addition, several of these transformation or transfection methods are equally suitable for ex vivo therapy. Furthermore, the tank2 polynucleotide sequences disclosed herein may be used in molecular biology techniques that have not yet been developed, provided the new techniques rely on properties of nucleotide sequences that are currently known, including but not limited to such properties as the triplet genetic code and specific base pair interactions.

Pharmaceutical Compositions

The present invention further relates to pharmaceutical compositions that comprise a chemical or biological compound (“agent”) that is active as a modulator of TANK2 expression or activity and a biocompatible pharmaceutical carrier, adjuvant, or vehicle. The active agent in the pharmaceutical compositions may be selected from among all or portions of tank2 polynucleotide sequences, tank2 antisense molecules, TANK2 polypeptides, protein, peptide, or organic modulators of TANK2 bioactivity, such as inhibitors, antagonists (including antibodies) or agonists. Preferably, the agent is active in treating a medical condition that is mediated by or characterized by TANK2 expression or activity. The composition can include the agent as the only active moiety or in combination with other nucleotide sequences, polypeptides, drugs, or hormones mixed with excipient(s) or other pharmnaceutically acceptable carriers.

Techniques for formulation and administration of pharmaceutical compositions may be found in Remington's Pharmaceutical Sciences, 18^{th} Ed., Mack Publishing Co., Easton, Pa. (1990). The pharmaceutical compositions of the present invention may be manufactured using any conventional method, e.g., mixing, dissolving, granulating, dragee-making, levigating, emulsifying, encapsulating. entrapping, melt-spinning, spray-drying, or lyophilizing processes. However, the optimal pharmaceutical formulation will be determined by one of skill in the art depending on the route of administration and the desired dosage. Such formulations may influence the physical state, stability, rate of in vivo release, and rate of in vivo clearance of the administered agent. Depending on the condition being treated, these pharmaceutical compositions may be formulated and administered systemically or locally.

The pharmaceutical compositions may be administered to the subject by any conventional method, including parenteral and enteral techniques. Parenteral administration modalities include those in which the composition is administered by a route other than through the gastrointestinal tract, for example, intravenous, intraarterial, intraperitoneal, intramedullary, intramuscular, intraarticular, intrathecal, and intraventricular injections. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments. Surgical techniques include implantation of depot (reservoir) compositions, osmotic pumps, and the like. A preferred route of administration for treatment of inflammation would be local or topical delivery for localized inflammation such as arthritis, and intravenous delivery for reperfusion injury or for systemic conditions such as septicemia.

The pharmaceutical compositions are formulated to contain suitable pharmaceutically acceptable carriers, and may optionally comprise excipients and auxiliaries that facilitate processing of the active compounds into preparations that can be used pharmaceutically. The administration modality will generally determine the nature of the carrier. For example, formulations for parenteral administration mav comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hank's solution, Ringer's solutions, or physiologically buffered saline. For tissue or cellular administration, penetrants appropriate to the particular barrier to be permeated are used in the formulation. Such penetrants are generally known in the art. For preparations cqmprising proteins, the formulation may include stabilizing materials, such as polyols (e.g., sucrose) and/or surfactants (e.g., nonionic surfactants), and the like.

Alternatively, formulations for parenteral use may comprise suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Emulsions, e.g., oil-in-water and water-in-oil dispersions, can also be used, optionally stabilized by an emulsifying agent or dispersant (surface-active materials; surfactants). Liposomes containing the active agent may also be employed for parenteral administration. Aqueous polymers that provide pH-sensitive solubilization and/or sustained release of the active agent may also be used as coatings or matrix structures, e.g., methacrylic polymers such as the Eudragit® series available from Röhm America Inc. (Piscataway, N.J.).

Alternatively, the pharmaceutical compositions comprising the agent in dosages suitable for oral administration can be formulated using pharmaceutically acceptable carriers well known in the art. The preparations formulated for oral administration may be in the form of tablets, pills, capsules, cachets, dragées, lozenges, liquids, gels, syrups, slurries, suspensions, or powders. To illustrate, pharmnaceutical preparations for oral use can be obtained by combining the active compounds with a solid excipient, optionally grinding the resulting mixture, and processing the mixture of granules, after adding suitable auxiliaries if desired, to obtain tablets or dragee cores. Note that oral formulations may employ liquid carriers similar in type to those described for parenteral use, e.g., buffered aqueous solutions, suspensions, and the like.

Preferred oral formulations include tablets, dragées, and gelatin capsules. These preparations may contain one or excipients, which include, without limitation:

a) diluents such as sugars, including lactose, dextrose, sucrose, mannitol, or sorbitol;

b) binders such as magnesium aluminum silicate, starch from corn, wheat, rice, potato, etc.;

c) cellulose materials such as methyl cellulose, hydroxypropylmethyl cellulose, and sodium carboxymethyl cellulose, polyvinyl pyrrolidone, gums such as gum arabic and gum tragacanth, and proteins such as gelatin and collagen;

d) disintegrating or solubilizing agents such as cross-linked polyvinyl pyrrolidone, starches, agar, alginic acid or a salt thereof such as sodium alginate, or effervescent compositions;

e) lubricants such as silica, talc, stearic acid or its magnesium or calcium salt, and polyethylene glycol;

f) flavorants, and sweeteners;

g) colorants or pigments, e.g., to identify the product or to characterize the quantity (dosage) of active compound; and

h) other ingredients such as preservatives, stabilizers, swelling agents, emulsifying agents, solution promoters, salts for regulating osmotic pressure, and buffers.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds may be dissolved or suspended in suitable fluids, such as fatty oils, liquid paraffin, or liquid polyethylene glycol with or without stabilizers.

Dragée cores can be provided with suitable coatings such as concentrated sugar solutions, which may also contain gum arabic, talc, polyvinyl pyrrolidone, carbopol gel, polyethylene glycol, and/or titanium dioxide, lacquer solutions, and suitable organic solvents or solvent mixtures.

The pharmaceutical composition may be provided as a salt of the active agent, which can be formed with many acids, including but not limited to hydrochloric, sulfuric, acetic, lactic, tartaric, malic, succinic, etc. Salts tend to be more soluble in aqueous or other protonic solvents that are the corresponding free base forms.

To be effective therapeutically in modulating central nervous system targets, the agents used in the methods of the invention should readily penetrate the blood brain barrier when peripherally administered. Compounds that cannot penetrate the blood brain barrier, however, can still be effectively administered by an intravenous route.

As noted above, the characteristics of the agent itself and the formulation of the agent can influence the physical state, stability, rate of in vivo release, and rate of in vivo clearance of the administered agent. Such pharmacokinetic and pharmacodynamic information can be collected through pre-clinical in vitro and in vivo studies, later confirmed in humans during the course of clinical trials. Thus, for any compound used in the method of the invention, a therapeutically effective dose can be estimated initially from biochemical and/or cell-based assays. Then, dosage can be formulated in animal models to achieve a desirable circulating concentration range that modulates TANK2 expression or activity. As human studies are conducted, further information will emerge regarding the appropriate dosage levels and duration of treatment for various diseases and conditions.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index,” which is typically expressed as the ratio LD₅₀/ED₅₀. Compounds that exhibit large therapeutic indices are preferred. The data obtained from such cell culture assays and additional animal studies can be used in formulating a range of dosage for human use. The dosage of such compounds lies preferably within a range of circulating concentrations that include the ED₅₀ with little or no toxicity.

For the method of the invention, any effective administration regimen regulating the timing and sequence of doses may be used. Doses of the agent preferably include pharmaceutical dosage units comprising an effective amount of the agent. As used herein, “effective amount” refers to an amount sufficient to modulate TANK2 expression or activity and/or derive a measurable change in a physiological parameter of the subject through administration of one or more of the pharmaceutical dosage units.

Exemplary dosage levels for a human subject are of the order of from about 0.001 milligram of active agent per kilogram body weight (mg/kg) to about 100 mg/kg. Typically, dosage units of the active agent comprise from about 0.01 mg to about 10,000 mg, preferably from about 0.1 mg to about 1,000 mg, depending upon the indication, route of administration, etc. Depending on the route of administration, a suitable dose may be calculated according to body weight, body surface area, or organ size. The final dosage regimen will be determined by the attending physician in view of good medical practice, considering various factors that modify the action of drugs, e.g., the agent's specific activity, the severity of the disease state, the responsiveness of the patient, the age, condition, body weight, sex, and diet of the patient, the severity of any infection, etc. Additional factors that may be taken into account include time and frequency of administration, drug combination(s), reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in human clinical trials. Appropriate dosages may be ascertained through use of established assays for determining concentration of the agent in a body fluid or other sample together with dose response data.

The frequency of dosing will depend on the pharmacokinetic parameters of the agent and the route of administration. Dosage and administration are adjusted to provide sufficient levels of the active moiety or to maintain the desired effect. Accordingly, the pharmaceutical compositions can be administered in a single dose, multiple discrete doses, continuous infusion, sustained release depots, or combinations thereof, as required to maintain desired minimum level of the agent. Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks. Pumps, such as subcutaneous, intraperitoneal, or subdural pumps, may be preferred for continuous infusion.

Compositions comprising a compound of the invention formulated in a pharmaceutical acceptable carrier may be prepared, placed in an appropriate container, and labeled for treatment of an indicated condition. Conditions indicated on the label may include treatment of inflammatory disorders, cancer, nervous tissue injury, etc. Kits are also contemplated, wherein the kit comprises a dosage form of a pharmaceutical composition and a package insert containing instructions for use of the composition in treatment of a medical condition.

The following Examples are provided to further aid in understanding the invention. The particular materials and conditions employed are intended to exemplify particular aspects of the invention and should not be construed to limit the reasonable scope thereof.

The Examples presuppose an understanding of conventional methods well-known to those persons having ordinary skill in the art to which the examples pertain, e.g., the construction of vectors and plasmids, the insertion of genes encoding polypeptides into such vectors and plasmids, or the introduction of vectors and plasmids into host cells. Such methods are described in detail in numerous publications including, for example, Sambrook et al., Molecular Cloning: A Laboratory Manual, Cold Spring Harbor Laboratory Press (1989), Ausubel et al. (Eds.), Current Protocols in Molecular Biology, John Wiley & Sons, Inc. (1994); and Ausubel et al. (Eds.), Short Protocols in Molecular Biology, 4^{th} ed., John Wiley & Sons, Inc. (1999).

### EXAMPLE 1

### Identification of an EST Related to Human Tankyrase1 and Isolation of a Tankyrase2 Polynucleotide

Using the nucleotide sequence of human tankyrase1 (SEQ ID NO:3) [Smith et al. (1998), supra], a search of the National Center for Biotechnology Information (NCBI) Expressed Sequence Tags (EST) database was performed to identify novel genes that are homologous to tankyrase1. The EST database provides 5′ and/or 3′nucleotide sequences for cDNA clones from a variety of tissue sources. The NCBI BLASTn program [Altschul et al., Nucleic Acids Res 25:3389-402 (1997)] was used to compare the nucleotide query sequence of human tankyrase1 against a nucleotide sequence database and to identify DNA sequences in the EST sequence database that have significant homology to human tankyrase1. This BLASTn search identified two EST sequences of interest: AA307492 (SEQ ID NO:5) cloned from a human colon carcinoma cell line designated HCC, and H17748 (SEQ ID NO:7), cloned from human brain.

A comparison of the AA307492 and tankyrase1 polynucleotides revealed that a region consisting of nucleotides 307 to 432 (nt 307-432) of AA307492 (SEQ ID NO:5) shared significant homology with a region consisting of nt 3313-3438 of tankyrase1 (SEQ ID NO:3); 105 of 126 nucleotides were the same; 83% identity). Nucleotides 307-432 of AA307492 were translated and the predicted protein (SEQ ID NO:6) was compared with tankyrase1 protein (amino acids 1105 to 1146 of SEQ ID NO:4). The proteins were found to be the same at 36 of 42 amino acid positions (86% identity). A comparison of the H 17748 and tankyrase1 polynucleotides revealed that nt 3-356 of H17748 (SEQ ID NO:7) shared significant homology with nt 3544-3897 of tankyrase1 (SEQ ID NO:3; 280 of 354 nucleotides were identical; 79% identity). When nt 3-356 of H17748 was translated and the predicted protein (SEQ ID NO:8) was compared with the corresponding region of tankyrase1 (aa 1182-1299 of SEQ ID NO:4), the proteins were found to be the same at 111 of 118 amino acid positions (94% identity). The putative amino acid sequences of AA307492 and H17748 are homologous to, but distinct from, tankyrase1 protein, indicating that they represented protein products translated from a novel tankyrase gene or genes.

AA307492 and H17748 were used in a search of the GenBank® database using the NCBI UniGene® program in order to identify other EST sequences originating from the same gene(s). The UniGene® program assembles GenBank sequences into a non-redundant set of gene-oriented clusters, with each cluster containing a group of sequences from the same gene. The UniGene® search of the human GenBank® database with AA307492 did not identify any other human EST sequences clustering in the same gene region as AA307492. By contrast, the UniGene® search of the human GenBank database with H17748 identified sixteen human EST sequences belonging in the same gene cluster as H17748, as follows: AA305587 (SEQ ID NO:9), AA371079 (SEQ ID NO:10), AA970617 (SEQ ID NO:11), AI247608 (SEQ ID NO:12), H11505 (SEQ ID NO:13), H11865 (SEQ ID NO:14), H17635 (SEQ ID NO:15), N29528 (SEQ ID NO:16), N57467 (SEQ ID NO:17), R06902 (SEQ ID NO:18), R06946 (SEQ ID NO:19), RI4158 (SEQ ID NO:20), R33944 (SEQ ID NO:21), R63031 (SEQ ID NO:22), R63337 (SEQ ID NO:23), and T17118 (SEQ ID NO:24). EST H17748 and EST H17635 contained sequence from opposite ends of the same clone, designated 50806. EST H11505 and EST H11865 contained sequence from opposite ends of the same clone, designated 47912. EST R06902 and EST R06946 contained sequence from opposite ends of the same clone, designated 126654. E. coli strains harboring cDNA clones 50806, 47912, and 126654 were purchased from the American Type Culture Collection (ATCC, Rockville, Md.), which maintains and makes publicly available deposits of ESTs identified and sequenced by I.M.A.G.E. (Lawrence Livermore National Laboratory, Livermore, Calif.). The three clones were sequenced as follows:

Clone 50806 was sequenced in its entirety on both strands using primers that hybridized to the vector DNA (SEQ ID NOs:25-26), and primers designed to hybridize to the human cDNA (SEQ ID NOs:27-34).

| | | |
|---|---|---|
| M13 Forward | TGTAAAACGACGGCCAGT | (SEQ ID NO:25) |
| M13 Reverse | GGAAACAGCTATGACCATG | (SEQ ID NO:26) |
| NT-7 | TTTGCCGGGTAACCTTGG | (SEQ ID NO:27) |
| NT-8 | CCAAGGTTACCCGGCAAA | (SEQ ID NO:28) |
| NT-9 | GTAGGCCCAGTGTAAATG | (SEQ ID NO:29) |
| NT-10 | CATTTACACTGGGCCTAC | (SEQ ID NO:30) |
| NT-11 | GAGTAAGTTGCAGGGCATGT | (SEQ ID NO:31) |
| NT-12 | ACATGCCCTGCAACTTACTC | (SEQ ID NO:32) |
| NT-13 | GAATCACCGCAGTTACTAAA | (SEQ ID NO:33) |
| NT-14 | TTTAGTAACTGCGGTGATTC | (SEQ ID NO:34) |

Clone 47912 was sequenced in its entirety on both strands using primers that hybridized to the vector DNA (SEQ ID NOs:25-26, supra), and primers designed to hybridize to the human cDNA (SEQ ID NOs:27-34, supra, and SEQ ID NOs:35-37).

| | | |
|---|---|---|
| NT-15 | GGCCTGAAGGTATGGTCGAT | (SEQ ID NO:35) |
| NT-16 | ATCGACCATACCTTCAGGCC | (SEQ ID NO:36) |
| NT-18 | TGAGGGCATTACAGTTTGTT | (SEQ ID NO:37) |

Clone 126654 was sequenced in its entirety on both strands using primers that hybridized to the vector DNA: M13 Forward (SEQ ID NO:25, supra) and T7 Promoter (SEQ ID NO:38), and primers designed to hybridize to the human cDNA (SEQ ID NOs:27-30, supra, and SEQ ID NOs:39-40).

| | | |
|---|---|---|
| T7 Promoter | TAATACGAACTCACTATAGGG | (SEQ ID NO:38) |
| NT-5 | ATACACTCACCGGAGAAA | (SEQ ID NO:39) |
| NT-6 | TTTCTCCGGTGAGTGTAT | (SEQ ID NO:40) |

Upon sequencing, 50806, 47912, and 126654 were found to be consistent with the sequences reported in the EST database. The polynucleotide sequences for 50806, 47912, and 126654 are set out in SEQ ID NOs:41, 43, and 45, respectively. The deduced amino acid sequences for 50806, 47912, and 126654 are set out in SEQ ID NOs:42, 44, and 46, respectively. The sequences of 50806 and 47912 indicated that the clones were identical, and only 50806 was considered further. 50806 and 126654 contain overlapping nucleotide sequence, but 126654 was 63 base pairs longer at the 5′ end, while 50806 was approximately 400 base pairs longer at the 3′ end.

50806 was determined to have an open reading (ORF) beginning at nucleotide position 1, a potential intron sequence at nt 358-1138, a stop codon beginning at nt 1999, and a potential poly A tail 474 base pairs 3′ to the stop codon. When nt 1-357 of 50806 were compared with nt 3538-3897 of tankyrase1, 283 of 357 nucleotides were the same (79% identical). When 50806 was translated from nt 1-357 and the resultant protein was compared with tankyrase1 (aa 1181-1299), the proteins were the same at 116 of 120 amino acid positions (97% identity).

A putative intron was identified in 50806, consisting of nt 358-1138, which may have been an artifact of cDNA cloning. DNA sequences preceding the putative intron (AG) and at the 3end of the putative intron (CAG) showed high resemblance to the consensus sequence for exon/intron/exon junctions [Lewin, GENES IV, Oxford University Press: New York (1997), at p. 88]. The most common sequence at the 3′ end of an exon is AG, and at the 3′ end of an intron is CAG. To determine if an intron is included in the 50806 sequence, PCR analysis of genomic DNA is used to verify this prediction.

A comparison of 50806 with tankyrase1 showed that a small region consisting of nt 1139-1198 of 50806 was significantly homologous with nt 3896-3957 of tankyrase1 (40 of 60 nucleotides were the same; 67% identity). When 50806 was translated from nt 1139-1198 and the resultant protein was compared with tankyrase1 (aa 1300 to 1319), the proteins were the same at 14 of 20 amino acid positions (70% identity). 126654 was determined to have an ORF beginning at nucleotide position 1, a stop codon beginning at position 481, and a potential poly A tail 81 base pairs 3′ of the stop codon. Comparison of 126654 with tankyrase1 showed that a region consisting of nt 1-480 of 126654 shared significant homology with nt 3478-3957 of tankyrase1 (367 of 481 nucleotides identical; 76% identity). When this region of 126654 was translated and the resultant protein compared with the corresponding region of the tankyrase1 protein (i.e., aa 1160-1319), the proteins were the same at 149 of 160 amino acid positions (97% identity). It is possible that either of the putative poly A tails of 50806 and 126654 were artifacts of cDNA cloning or that 50806 and 126654 represented a population of mRNA that use different polyadenylation sites. 50806 had a stretch of 8 A residues 81 base pairs 3′ to the stop codon, indicating that the putative poly A tail of 126654 was most likely a cloning artifact.

Alignment of AA307492 and 126654 with human tankyrase1 using the Sequencher™ program (Gene Codes Corporation, Ann Arbor, Mich.) suggested that AA307492 was upstream of 126654, and that 11 nucleotides separated AA307492 and 126654. To confirm that AA307492 and 126654 represented polynucleotide sequence from the same gene, a primer (SEQ ID NO:47) corresponding to the sense strand of AA307492 and a primer (SEQ ID NO:48) corresponding to the antisense strand of 126654 were synthesized for use in a polymerase chain reaction (PCR) with human Marathon®-Ready spleen and testis cDNA (Clontech) as the template.

| | | |
|---|---|---|
| AA307492 | CTCCGGACAACAAGGTCTTAACC | (SEQ ID NO:47) |
| sense | | |
| 126654 | CCACCTATGTACGCATGCC | (SEQ ID NO:48) |
| antisense | | |

The PCR reaction contained 2.5 μL human spleen Marathon®-Ready cDNA, 2.5 μL human testis Marathon-Ready cDNA, 250 nM each primer, 0.25 mM dNTPs, 1×PCR buffer, 1.8 mM MgCl₂, and 5 Units of Taq polymerase (Perkin Elmer). The reaction was performed in a GeneAmp® PCR System 9700 machine (hereinafter “GeneAmp® PCR System 9700”; PE Applied Biosystems, Norwalk Conn.) and first heated at 94° C. for 2 min, followed by 35 cycles of 94° C. for 30 sec, 55° C. for 30 sec, and 72° C. for 30 sec, and ended with 7 min at 72° C. The PCR fragment was isolated using gel electrophoresis and a QIAquick® Gel Extraction Kit (hereinafter “QIAquick® kit”; Qiagen, Valencia, Calif.), according to the manufacturer's instructions. The PCR fragment was directly cloned into pCR®2.1 -TOPO® vector (Invitrogen, Carlsbad, Calif.), according to the manufacturer's instructions. The PCR fragment was sequenced with primers that hybridized to the vector DNA (SEQ ID NOs:25 and 26, supra), and the sequence of the AA307492/126654 PCR fragment is set out in SEQ ID NO:49. The sequence confirmed that AA307492 was upstream of 126654 and that these two ESTs were separated by 1I nucleotides, and that AA307492 and 126654 were sequences from a novel gene, designated tankyrase2.

To identify the full-length tankyrase2 gene, a probe was generated from 126654 and used to screen a cDNA library using procedures routinely practiced in the art. 126654 was digested with XhoI and BglII, and an approximately 260 nucleotide fragment designated NT-5′ was isolated using gel electrophoresis and the QlAquick(® kit. NT-5′ was labeled with ³²P with a Random Primed DNA Labeling Kit (Boehringer Mannheim/Roche Molecular Biochemicals, Indianapolis, Ind.) according to the manufacturer's instructions and used to screen 10⁶ cDNAs from a human fetal brain library (Stratagene). Hybridization with labeled probe was performed overnight at 65° C. in buffer containing: 3×SSC, 0.1% sarkosyl, 20 mM sodium phosphate, pH 6.8, 10×Denhardt's solution, and 50 μg/mL salmon sperm DNA. The filters were washed at 65° C. in buffer containing 2×SSC and 0.1% SDS prior to autoradiography. Forty-six positives were obtained with the NT-5′ probe, of which fifteen were first characterized with respect to strength of hybridization with NT-5′. Restriction digest mapping and partial sequencing led to the selection of two clones, designated FB2B.1 and FB2D. 1, for further characterization.

FB2B.1 was sequenced in its entirety on both strands with primers that hybridized to the vector DNA, including T7 promoter (SEQ ID NO:38, supra) and T3 promoter (SEQ ID NO:50), and primers designed to anneal to the cDNA sequence (SEQ ID NOs:51-69).

| | | |
|---|---|---|
| T3 promoter | ATTTAACCCTCACTAAAGGG | (SEQ ID NO:50) |
| 2B.1 F1 | AAAGGCTCCCATCGGCAAAT | (SEQ ID NO:51) |
| 2B.1 F2 | GTTGAGGGCATTACAGTTTG | (SEQ ID NO:52) |
| 2B.1 F3 | AAAACGTAGAGGCCACTGCT | (SEQ ID NO:53) |
| 2B.1 F4 | TGGTGTAGACTGACGCCCTT | (SEQ ID NO:54) |
| 2B.1 F5 | TCCGGTGAGTGTATCTTTCC | (SEQ ID NO:55) |
| 2B.1 F6 | CTCCTTTGTCTTGGGCATTC | (SEQ ID NO:56) |
| 2B.1 F9 | ATCTGCTCTGCCCTCTTGTT | (SEQ ID NO:57) |
| 2B.1 F10 | GGGTATCGCGGCAATTTACA | (SEQ ID NO:58) |
| 2B.1 F11 | AACAAGAGGGCAGAGCAGAT | (SEQ ID NO:59) |
| 2B.1 F12 | TGCCCCATCTCAACTAATAC | (SEQ ID NO:60) |
| 2B.1 R2 | GTAATGCCCTCAACAGAACT | (SEQ ID NO:61) |
| 2B.1 R3 | GGCGTCAGTCTACACCACTT | (SEQ ID NO:62) |
| 2B.1 R4 | TAAATTGCCCGCGATACCCA | (SEQ ID NO:63) |
| 2B.1 R5 | CACTCAGTCACTGGTAGGCC | (SEQ ID NO:64) |
| 2B.1 R6 | ATCTGCTCTGCCCTCTTGTT | (SEQ ID NO:65) |
| 2B.1 R7 | TAGTTGAGATGGGGCACAAG | (SEQ ID NO:66) |
| 2B.1 R8 | AAACGTAGAGGCCACTGCTG | (SEQ ID NO:67) |
| 2B.1 R9 | CGGGTAACCTTGGGAAAGTC | (SEQ ID NO:68) |
| 2B.1&2D.1 | GGGCTTTACTGCTTTACAGA | (SEQ ID NO:69) |

FB2D.1 was sequenced in its entirety on both strands with primers that hybridized to the vector DNA (SEQ ID NOs:38 and 50, supra) and primers designed to anneal to the cDNA sequence, including 2B.1&2D.1 (SEQ ID NO:69) and SEQ ID NOs:70-87.

| | | |
|---|---|---|
| 2D.1 F1 | GTAAGGGCTGCTGACAGTGA | (SEQ ID NO:70) |
| 2D.1 F2 | TTACTCCAGCAGAGGGCACT | (SEQ ID NO:71) |
| 2D.1 F3 | CTGACGCCCTTCAATGTCTC | (SEQ ID NO:72) |
| 2D.1 F4 | GGTACTAAGGCCACAATTCA | (SEQ ID NO:73) |
| 2D.1 F5 | GGGTATCGCGGCAATTTACA | (SEQ ID NO:74) |
| 2D.1 F6 | GTTGAGGGCATTACAGTTTG | (SEQ ID NO:75) |
| 2D.1 F7 | TAACAAGAGGGCAGAGCAGA | (SEQ ID NO:76) |
| 2D.1 F8 | AGTTCTGTTGAGGGCATTAC | (SEQ ID NO:77) |
| 2D.1 F9 | GGCCTACCAGTGACTGAGTG | (SEQ ID NO:78) |
| 2D.1 F10 | GGGCTAGAGGACCTGAAGAG | (SEQ ID NO:79) |
| 2D.1 R2 | AGTGCCCTCTGCTGGAGTAA | (SEQ ID NO:80) |
| 2D.1 R3 | GGCGTCAGTCTACACCACTT | (SEQ ID NO:81) |
| 2D.1 R4 | TGAATTGTGGCCTTAGTACC | (SEQ ID NO:82) |
| 2D.1 R5 | ATGCCCAAGACAAAGGAGGA | (SEQ ID NO:83) |
| 2D.1 R6 | GTAATGCCCTCAACAGAACT | (SEQ ID NO:84) |
| 2D.1 R7 | ATCTGCTCTGCCCTCTTCTT | (SEQ ID NO:85) |
| 2D.1 R8 | CGGGTAACCTTGGGAAAGTC | (SEQ ID NO:86) |
| 2D.1 R9 | CCGGACAACAAGGTCTTAAC. | (SEQ ID NO:87) |

The polynucleotide sequences for FB2B.1 and FB2D.1 are set out in SEQ ID NOs:88 and 90, respectively, and the deduced amino acid sequences of FB2B. 1 land FB2D. 1 are set out in SEQ ID NOs:89 and 91, respectively.

The nucleotide and amino acid sequences of FB2B.1 and tankyrase1 were compared to determine the degree of relatedness between the sequences. A region consisting of nt 4-279 of FB2B.1 (SEQ ID NO:88) was found to have significant identity with nt 1624-1899 of tankyrase1 (SEQ ID NO:3), wherein 203 of 276 nucleotides were identical (73% identity). Nucleotides 402-1254 of FB2B.1 showed significant identity with nt 2022-2874 of tankyrase1, wherein 630 of 853 nucleotides were identical (73% identity). Furthermore, nt 1507-2338 of FB2B.1 showed homology to nt 3112-3943 of tankyrase1, wherein 634 of 832 nucleotides were identical (76% identity). FB2B.1 was determined to have an ORF beginning at nucleotide position 1, a stop codon beginning at position 2353, approximately 1 kb of 3′ untranslated sequence, but no apparent poly A tail. A translation of nt 1-2352 of FB2B.1 showed that a region consisting of the predicted amino acid sequence (SEQ ID NO:89) was homologous to a corresponding region of tankyrase1 (aa 540-1327 of SEQ ID NO:4). In this region, the proteins were identical at 623 of 777 amino acid positions (80% identity).

A similar comparison of FB2D.1 was made with tankyrase1. In this case, a region consisting of nt 6-197 of FB2D.1 (SEQ ID NO:90) was significantly related to nt 1708-1899 of tankyrasel, wherein 137 of 192 nucleotides were identical (71% identity). Nucleotides 320-1172 of FB2D.1 were found to share significant homology with corresponding nt 2022-2874 of tankyrase1, wherein 630 of 853 nucleotides were identical (73% identity). Nucleotides 1425-2256 of FB2D.1 showed significant homology with nt 3112-3943 of tankyrase1, wherein 634 of 832 nucleotides were identical (76% identity). FB2D.1 was determined to have an ORF beginning at nucleotide position 3, a stop codon beginning at position 2271, approximately 1.5 kb of 3′ untranslated sequence, but no apparent poly A tail. When FB2D.1 was translated (SEQ ID NO:91), a domain predicted by the nt 3-2270 showed homology to aa-569-1327 of tankyrase1 (SEQ ID NO:4). Here, the proteins were the same at 602 of 749 amino acid positions (80% identity).

FB2B.1 and FB2D.1 were aligned using Sequencher™. FB2B.1 and FB2D.1 contained overlapping polynucleotide sequence, but FB2B.1 was longer at the 5′ end by 82 base pairs, and FB2D.1 was longer at the 3′ end by approximately 0.5 kb. The nucleotide sequences of FB2B.1 and FB2D.1 were identical in the regions nt 83-2971 of FB2B.1 and nt 1-2889 of FB2D.1. However, the remaining 382 nucleotides of FB2B.1 and 910 nucleotides of FB2D.1 did not align. It is possible that FB2B.1 and FB2D.1 were random primed from different positions in the 3′ untranslated region and/or that this misalignment was the result of the presence of a cloning artifact in one or both of the clones. Since FB2B.1 and FB2D. I did not appear to have poly A tails, the poly A tails of ESTs 50806 and 126654 were most likely cloning artifacts, and the real poly A tail of tankyrase2 was most likely greater than 0.5 kb from the stop codon. A consensus polynucleotide sequence, designated 2B.½D.1, was developed from the alignment of FB2B.1 and FB2D.1, and is set out in SEQ ID NO:92. 2B.l1/2D.1 contained nt 1-2971 of FB2B.1 and nt 1-2889 of FB2D.1.

Alignment of FB2B.1 and FB2D.1 with tankyrase1 using Sequencher™ suggested that neither FB2B. I nor FB2D.1 represented a full-length gene, and that nucleotide sequence was missing from the 5′ end of tankyrase2. Thus, FB2B.1 was digested with EcoRI and SphI, and an approximately 466 bp nucleotide fragment located at the immediate 5′ end of FB2B.1 (nt 49-515 of SEQ ID NO:88) was isolated using gel electrophoresis and the QIAquick® kit. This fragment was labeled with ³²P with a Random Primed DNA Labeling Kit and used as a probe (designated NT-37/38) to screen 10⁶ cDNA clones of the fetal brain library (Stratagene) using the conditions and procedures used in the first screening. Fourteen positives were obtained with the NT-37/38 probe, one of which (designated 30B.2A) also hybridized with the NT-5′ probe, but which had not been chosen for further characterization at that time. Restriction mapping and partial sequencing led to the selection of 30B.2A for further characterization.

The region of 30B.2A upstream of clone FB2B.1 was sequenced with primers that hybridized to the vector DNA (SEQ ID NOs:38 and 50, supra) and primers designed to anneal to the cDNA sequence, including 2B.1 F4 (SEQ ID NO:54. supra) and SEQ ID NOs:93-97).

| | | |
|---|---|---|
| 30B.2A #1 | GGGCGGAAAGACGTAGTTGA | (SEQ ID NO:93) |
| 30B.2A #2 | GCGGCTGTTCACCTTCTCAG | (SEQ ID NO:94) |
| 30B.2A #5 | ACGCAAGTGATGGCAGAAAG | (SEQ ID NO:95) |
| 30B.2A #6 | TCACTTGCGTGGCAGTTGAC | (SEQ ID NO:96) |
| 30B.2A #7 | GCGGCAGGTTTGTAGATGAC | (SEQ ID NO:97) |

The partial polynucleotide sequence of 30B.2A is set out in SEQ ID NO:98, and the partial deduced amino acid sequence is set out in SEQ ID NO:99. Comparison of 30B.2A with the nucleotide sequence oftankyrase1 indicated that significant homology occurred in the region consisting of nt 167-1435 of 30B.2A which corresponded with nt 631-1899 of tankyrase1. In this region, 953 of the 1269 nucleotides were the same (75% identity). 30B.2A was determined to have an ORF beginning at nucleotide position 2. Significant amino acid sequence identity was observed between a 385 amino acid sequence predicted for 30B.2A (based on nt 2-1156) and the corresponding region of tankyrase1 (aa 160-539). In this region, the protein sequences were the same at 319 of 385 amino acid positions (83% identity).

2B.1/2D.1 and 30B.2A were aligned using Sequencher™. 30B.2A 2A contained 1.157 kb of novel sequence before it began overlapping with the 5′ end of 2B.1/2D.1, and began overlapping with 2B.½D.1 at position 1158. A consensus polynucleotide sequence, designated 2B.½D.1/30B.2A, was developed from the alignment of 2B.½D.1 and 30B.2A, and is set out in SEQ ID NO:100. 2B.½D.1/30B.2A contained nt 1-1157 of 30B.2 and nt 1-2971 of 2B.½D.1. The predicted amino acid sequence encoded by nt 2-3508 of SEQ ID NO:100 is set forth as SEQ ID NO:101. The nucleotide sequence ofthe TANK2-encoding region is set forth as SEQ ID NO:1, and the corresponding TANK2 polypeptide sequence is set forth as SEQ ID NO:2.

### EXAMPLE 2

### Cloning of 5′ End of Tankyrase2

Alignment of 30B.2A with tankyrase1 using the Sequencher™ program suggested that 5′ sequence was still lacking from the tankyrase2 gene. To clone the 5′ end of human tankyrase2, 5′ RACE analysis was performed using a Marathon(®-Ready human spleen cDNA library (Clontech) as the template. A primer (NT-Marathon; SEQ ID NO:102) corresponding to the antisense strand of 2B.½D.1/30B.2A polynucleotide sequence (nt 337-367 of SEQ ID NO:100) was synthesized for use in a polymerase chain reaction (PCR) with the AP1 primer (Clontech; SEQ ID NO:103) that was designed to anneal to the Marathon(® cDNA Adapters ligated to the ends of the cDNAs in the library.

| | |
|---|---|
| NT-Marathon | |
| GAGCATTGGGGTCTGCACCATGTCGCAAAAGG | (SEQ ID NO:102) |
| AP1 | |
| CCATCCTAATACGACTCACTATAGGGC | (SEQ ID NO:103) |

The PCR reaction contained 5 μL human spleen Marathon®-Ready cDNA, 0.20 μM each primer, 0.20 mM dNTPs, 1×Clontech GC 2 PCR buffer, Clontech GC-Melt buffer (0, 0.5, 1.0, or 1.5 M), and 1 μL of Clontech Advantage®-GC 2 polymerase mix. The reactions were performed in a GeneAmp® PCR System 9700 with the following four steps: 1) 1 cycle at 94° C. for 1 min; 2) 5 cycles of 94° C. for 30 sec and 72° C. for 30 sec; 3) 5 cycles of 94° C. for 30 sec and 70° C. for 30 sec; and 4) 25 cycles of 94° C. for 30 sec and 60° C. for 30 sec. The reactions were then continued in the GeneAmp® PCR System 9700 under the following conditions: 1) 1 cycle at 94° C. for 1 min; 2) 5 cycles of 94° C. for 30 sec, and 72° C. for 3 min; 3) 5 cycles of 94° C. for 30 sec and 70° C. for 3 min; and 4) 25 cycles of 94° C. for 30 sec and 60° C. for 3 min. The PCR fragments were isolated using gel electrophoresis and a QIAquick® kit as directed. The PCR fragments were directly cloned into the pCR®2.1-TOPO® vector, as directed. Because Taq polymerase has an error rate of 8.0×10⁻⁶ mutation/base pair (Cline et al., Nucleic Acids Res 24:3546-51), four clones isolated from four separate PCR reactions were sequenced and compared to eliminate the possibility of Taq polymerase-induced errors in the 5′ RACE sequences. The four 5′ RACE clones were sequenced with the M13 forward and M13 reverse primers (SEQ ID NOs:25 and 26) that hybridize to the vector DNA. The four individual nucleotide sequences were compiled into a consensus nucleotide sequence designated 5′-RACE tank2 that is set out in SEQ ID NO:104, and the deduced amino acid sequence is set out in SEQ ID NO:105. In the consensus nucleotide sequence of 5′-RACE tank2, every base pair was present at the corresponding position in at least three of the four unique clones used to compile the consensus sequence. 5′-RACE tank2 and tankyrase were aligned using the Sequencher™ program. When nt 1-279 of 5′-RACE tank2 (SEQ ID NO:104) were compared with tankyrase no significant similarity was found. 5′-RACE tank2 was determined to have an ORF beginning at nucleotide position 2. When nt 2-277 of 5′-RACE tank2 was translated and the resultant protein was compared with tankyrase, no significant similarity was found.

5′-RACE tank2 and 2B.½D.1/30B.2A were aligned using the Sequencher™ program. 5′-RACE tank2 contained 279 bp of novel sequence before it began overlapping with the 5′ end of FB2B.½D.1/30B.2A, and began overlapping with 2B.½D.1/30B.2A at position 280. A consensus polynucleotide sequence designated 2B.½D.1/30B.2A/5′-RACE, was developed from the alignment of 5′-RACE tank2 and 2B.½D.1/30B.2A and is set out in SEQ ID NO:106. 2B.½D.130B.2A/5′-RACE contained nt 1-279 of 5′-RACE tank2 and nt 1-4140 of 2B.½D.1/30B.2A. The deduced putative amino acid sequence of 2B.½D.1/30B.2A/5′-RACE is set out in SEQ ID NO:107.

The presence of a continuous ORF in the 5′-RACE tank sequence suggested that 5′ sequence was still lacking from the tankyrase2 gene. Further attempts to obtain additional 5′ sequence of tankyrase2 using 5′ RACE analysis were unsuccessful. The NCBI BLASTn program was used to compare the nucleotide query sequence of FB2B.½D.1/30B.2A against a nucleotide sequence tag database (a non-redundant database of GenBank®+EMBL+DDBJ STS Divisions). This BLASTn search identified a STS tag sequence designated stWI-16054 (GenBank® Accession No. G24639; SEQ ID NO:108). When nt 3608-3985 of 2B.½D.1/30B.2A was compared with the antisense complement nt 8-397 of stWI-16054, 361 of 378 nucleotides were the same (96% identical). The Sanger Centre (Cambridge, UK) Human Genome Clone Search program (http:wwww.sanger.ac.uk/vegi-bin/humace/searcher.egi) was used to identify BAC clones containing stWI-16054. BAC clone bA329B8 was identified as containing the STS tag stWI-16054. BAC clone bA329B8 originates from the genomic RPCI-11.2 male white blood cell library (Pieter deJong, Roswell Park Cancer Institute, Buffalo, N.Y.) and was purchased from Research Genetics, Inc. (Huntsville, Ala.). A Large Construct Kit (Qiagen) was used to isolate bA329B8 DNA, which was used as a template in inverse PCR amplification reactions [Ochman et al., “Amplification of Flanking Sequences by Inverse PCR,” pp. 219-27 in PCR Protocols: A Guide to Methods and Applications (Innis et al., eds.), Academic Press, San Diego, Calif. (1990)]. The inverse PCR technique allows for the amplification of unknown DNA sequence flanking a region of known sequence. Briefly, template DNA is digested with a restriction enzyme (preferably, one that recognizes a four or five base pair consensus site), followed by circularization of the restriction fragments. Circularized fragments are used as a template in a PCR reaction with two primers designed to anneal to the known flanking sequence but pointed in opposite directions. One microgram (1 μg) of bA329B8 was digested in a 20 μL reaction containing 1× appropriate reaction buffer and 10 units of one of the following restriction enzymes: RsaI (Promega, Madison, Wis.), BfaI (New England Biolabs, Beverly, Mass.), or Tri9I (Promega). The restriction digests were incubated for one hour at 37° C. (RsaI and BfaI) or 65° C. (Tru9I). The RsaI and BfaI digests were heated at 68° C. for 20 minutes to inactivate the restriction enzymes. A QIAquick® kit was used to inactivate the restriction enzyme in the Tru9I digest. Ligation reactions contained the following: 20 μL of the Tru9I, RsaI, or BfaI reactions, 448 μL distilled water, 50 μL 10× reaction buffer, and 2 μL T4 DNA ligase (5U/μL; Boehringer Mannheim, Indianapolis, Ind.). Ligations were incubated overnight at 15° C. The DNAs in the ligation reactions were then precipitated by adding 129.26 μL 7 M ammonium acetate and 2.3 mL 95% ethanol. The DNAs were pelleted, washed with 75% ethanol, resuspended in 15 μL distilled water, and used as templates in PCR amplification reactions. A primer (5-Inv-1; SEQ ID NO:109) corresponding to the sense strand of 5′-RACE tank2 (nt 423-443 of SEQ ID NO:104) and a primer (3-Inv-1; SEQ ID NO:110) corresponding to the antisense strand of 5′-RACE tank2 (nt 364-383 of SEQ ID NO:104) were synthesized for use in PCR amplification reactions.

| | | |
|---|---|---|
| 5-Inv-1 | CGCCTGAGAAGGTGAACAGCC | (SEQ ID NO:109) |
| 3-Inv-1 | ACGCCTCGAACAGCTCTCGG | (SEQ ID NO:110) |

The PCR reactions (final reaction volume of 20 μL) contained 5 μL of the Tru9I, RsaI, or BfaI DNA template, 0.20 μM each primer, 0.20 mM dNTPs, 1×Clontech GC 2 PCR buffer, 1.0 M Clontech GC-Melt buffer, and 0.4 μL of Clontech Advantage®-GC 2 polymerase. The reactions were performed in a GeneAmp® PCR System 9700 with the following four steps: 1) 1 cycle at 94° C. for 1 minute; 2) 5 cycles of 94° C. for 30 seconds and 65° C. for 3 minutes and 30 seconds; 3) 5 cycles of 94° C. for 30 seconds and 60° C. for 3 minutes and 30 seconds; and 4) 25 cycles of 94° C. for 30 seconds and 58° C. for 3 minutes and 30 seconds. The PCR fragments were isolated using gel electrophoresis and a QIAquick® kit as directed. The PCR fragments were directly cloned into the pCR®2.1-TOPO® vector, as directed. The Tru9I, RsaI, and BfaI clones were sequenced with the M13 primers that hybridize to the vector DNA (SEQ ID NOs:25 and 26) and primers designed to anneal to the cDNA sequence (SEQ ID NOs:109-112).

| | | |
|---|---|---|
| 5-Inv-2 | GCGTGGGCGCGGCCATGGGACTG | (SEQ ID NO:111) |
| 3-Inv-2 | CAGCGCGAATCCGCCGTCCG | (SEQ ID NO:112) |

The Tru9I, RsaI, and BfaI polynucleotide sequences are set out in SEQ ID NOs:113, 115, and 117, respectively. The deduced amino acid sequences of Tru9I, RsaI, and BfaI are set out in SEQ ID NOs:114, 116, and 118, respectively.

Clones Tru9I and 5′-RACE tank2 were aligned using the Sequencher™ program. Clone Tru9I (SEQ ID NO:113) contained 235 bp of novel sequence before it began overlapping with the 5′ end of 5′-RACE tank2 (SEQ ID NO:104), and began overlapping with 5′-RACE tank2 at position 236. When nt 1-235 of clone Tru9I were compared with tankyrase no significant similarity was found. Clone Tru9I was determined to have an ORF beginning at nucleotide position 3. When clone Tru9I was translated from nt 3-236 and the resultant protein was compared with tankyrase no significant similarity was found.

Clone RsaI and 5′-RACE tank2 were aligned using the Sequencher™ program. Clone RsaI (SEQ ID NO:115) contained 654 bp of novel sequence before it began overlapping with the 5′ end of 5′-RACE tank2 (SEQ ID NO:104). and began overlapping with 5′-RACE tank2 at position 655. When nt 1-654 of clone Rsal were compared with tankyrase no significant similarity was found. Clone RsaI was determined to have an ORF beginning at nucleotide position 160, with a putative ATG start codon beginning at nucleotide 287. When clone RsaI was translated from nt 287-655 and the resultant protein was compared with tankyrase no significant similarity was found.

Clone BfaI (SEQ ID NO:117) and 5′-RACE tank2 were aligned using the Sequencher™ program. Clone BfaI contained 88 bp of novel sequence before it began overlapping with the 5′ end of 5′-RACE tank2 (SEQ ID NO:104), and began overlapping with 5′-RACE tank2 at position 89. When nt 1-88 of clone BfaI were compared with tankyrase no significant similarity was found. Clone BfaI was determined to have an ORF beginning at nucleotide position 3. When clone BfaI was translated from nt 3-89 and the resultant protein compared with tankyrase no significant similarity was found.

To confirm the new polynucleotide sequence obtained from the Tru9I, RsaI, and BfaI clones and to determine if introns are present in the new sequence, PCR amplification of cDNA was performed. A primer (5-RSA-1; SEQ ID NO:119) corresponding to the sense strand of clone RsaI (nt 59-84 of SEQ ID NO:115) and a primer (3-Inv-1; SEQ ID NO:110) corresponding to the antisense strand of clone RsaI (nt 708-727 of SEQ ID NO:115) were synthesized for use in PCR amplification reactions.

5-RSA-1 GTTCCTCTAATCAATCCTGAGC (SEQ ID NO:119) Six separate PCR reactions were performed (designated 18, 19, 20, 24, 25, and 26) to aid in the identification of Taq polymerase-induced errors as described above. Each 20 μL reaction contained 5 μL of human spleen, placenta, or testis Clontech Marathon®-Ready cDNA DNA template, 0.20 μM each primer, 0.20 mM dNTPs, 1×Clontech GC 2 PCR buffer, 1.0 M Clontech GC-Melt buffer, and 0.4 μL of Clontech Advantage®-GC 2 polymerase. The reactions were performed in a GeneAmp® PCR System 9700 with the following four steps: 1) 1 cycle at 94° C. for 1 min; 2) 5 cycles of 94° C. for 30 sec and 65° C. for 2.5 min; 3) 5 cycles of 94° C. for 30 sec and 60° C. for 2.5 min; and 4) 25 cycles of 94° C. for 30 sec and 58° C. for 2.5 min. The PCR fragments were isolated using gel electrophoresis and a QIAquick® kit as directed. The PCR fragments were directly cloned into the pCR®2.1 -TOPO® vector, as directed. Clones 18, 19, 20, 24, 25, and 26 were sequenced with the M13 primers that hybridized to the vector DNA (SEQ ID NOs:25 and 26) and primers designed to anneal to the cDNA sequence (SEQ ID NOs:112, 120, 121, and 122).

| | |
|---|---|
| 5-RSA-2 | |
| GGAAAGAGTAATTGATCAGAGCCATC | (SEQ ID NO:120) |
| 5-RSA-4 | |
| CGCCGAAGCCTCTCGCCTCACATTTCC | (SEQ ID NO:121) |
| 3-RSA-4 | |
| GGAAATGTGAGGCGAGAGGCTTCGGCG | (SEQ ID NO:122) |

The polynucleotide sequences of clones 18, 19, 20, 24, 25, and 26 are set out in SEQ ID NOs:123-128, respectively.

Clones 18, 19, 20, 24, 25, 26 and clone RsaI were aligned using the Sequencher™ program. The polynucleotide sequence of the cDNA clones confirmned that there were no introns present in the RsaI clone sequence. Base pairs 1-596 of clones 18, 19, 20, 24, 25, and 26 were compiled into a consensus nucleotide sequence with bp 59-596 of clone RsaI that is designated 5′-RSA/cDNA and is set out in SEQ ID NO:129. The polynucleotide sequence of 5′-RSA/cDNA does not include nucleotide sequence 3′ to base pair 597 of clones 18, 19, 20, 24, 25, 26, which is discussed below. The polynucleotide sequence of 5′-RSA/cDNA also does not include bp 1-58 of clone RsaI, as this nucleotide sequence was not confirmed in the cDNA clone sequence. In the consensus nucleotide sequence of 5′-RSA/cDNA, every base pair was present at the corresponding position in 6 of the 7 clones, except nucleotide position 47 in which the consensus base pair was present at the corresponding position in 4 of the 7 clones.

The alignment of clones 18, 19, 20, 24, 25, and 26 identified a difference in the nucleotide sequence 3′ to base pair 597 (reference position in SEQ ID NOs:123-128). All of the aligned clones contain one copy of a 10 base pair sequence (GAGCTGGCAG; SEQ ID NO:130) located at nt 588-597 (SEQ ID NOs:123-128). Clones 19 and 26 have a second copy of the sequence GAGCTGGCAG repeated directly adjacent to the first copy (nt 598-607) (SEQ ID NOs: 124 and 128). Clone RsaI, clone Tru9I, and clone BfaI also have two copies of the sequence GAGCTGGCAG directly adjacent to each other (nt 646-665 in clone RsaI (SEQ ID NO:115); nt 227-246 in clone Tru9I (SEQ ID NO:113); and nt 80-99 in clone BfaI (SEQ ID NO:117)). Clones 18, 20, 24, and 25 do not have the second copy of the sequence GAGCTGGCAG. The presence or absence of the second copy of the sequence GAGCTGGCAG could result from an error in PCR amplification caused by Taq polymerase. Direct sequencing of genomic DNA can be used to verify this prediction. The presence or absence of the second copy of the sequence GAGCTGGCAG could also be caused by replication and/or repair proteins present in the bacteria used to propagate the cloned DNA. Direct sequencing of PCR products can be used to verify this prediction. The presence or absence of the second copy of the sequence GAGCTGGCAG could also result from alternative 3′-splice acceptor usage. This possibility seems unlikely since the sequences surrounding the GAGCTGGCAG sequence do not show high resemblance to the consensus sequence for exon/intron/exon borders [Lewin, supra]. In addition, clones generated from PCR amplification of genomic DNA have been isolated that contain only one copy of the GAGCTGGCAG sequence (Genomic 1 X; SEQ ID NO:131) as well as clones containing two copies of the GAGCTGGCAG sequence (clones RsaI (SEQ ID NO:115) Tru9I (SEQ ID NO:113) and BfaI (SEQ ID NO:117)). The presence or absence of the second copy of the sequence GAGCTGGCAG may also be a polymorphism present in the human population. In this case, expression of a long and short form of the TANK2 protein would be possible, as discussed below.

The presence of two copies of the sequence GAGCTGGCAG produces a long form of the TANK2 protein. Clones 19, 26, RsaI, Tru9I, and Bfal were aligned with 5′-RSA/cDNA and 2B.½D.1/30B.2A/5′-RACE using the Sequencher™ program. A consensus polynucleotide sequence designated tankyrase2-long was developed from the alignment and is set out in SEQ ID NO:132. The sequence of tankyrase2-long was determined to have an ORF from nt 103-4386, with the first methionine beginning at nt 229. An in-frame stop codon (beginning at nt 100) was present upstream of the putative initiating methionine. Assuming that this residue is the initiating methionine, the ORF of tankyrase2-long encodes a protein of 1385 amino acids (designated TANK2-LONG; SEQ ID NO:133) with a predicted molecular weight of 149,892 Da.

The presence of one copy of the sequence GAGCTGGCAG produces a short form of the TANK2 protein. Clones 18, 20, 24, and 25 were aligned with 5′-RSA/cDNA and 2B.½D.1/30B.2A/5′-RACE using the Sequencher™ program. A consensus polynucleotide sequence designated tankyrase2-short was developed from the alignment and is set out in SEQ ID NO:134. The sequence of tankyrase2-short was determined to have an ORF from nt 513-4376, with the first methionine beginning at nt 876. An in frame stop codon (beginning at nt 510) was present upstream of the putative initiating methionine. Assuming this residue to be the initiating methionine, the ORF of tankyrase2-short encoded a 1166 amino acid protein (designated TANK2-SHORT; SEQ ID NO:135) with a predicted molecular weight of 126,908 Da. TANK2-SHORT is 219 amino acids shorter at the amino terminal end than TANK2-LONG. The putative initiating methionine of TANK2-SHORT corresponds to a methionine at position 120 of TANK2-LONG. Excluding the first 219 amino acids of TANK2-LONG, TANK2-LONG and TANK2-SHORT are identical.

The tankyrase1 gene (SEQ ID NO:3) encodes a protein TANK1 (SEQ ID NO:4) containing a carboxyl-terminal catalytic domain that has homology to the catalytic domain of human PARP1. The polynucleotide sequence of parp 1 is set out in SEQ ID NO:136, and the amino acid sequence of PARP 1 is set out in SEQ ID NO:137. The catalytic domain of TANK1 (aa 1176-1314 of SEQ ID NO:4) is homologous to the catalytic domain of PARP1 (aa 854-1014 of SEQ ID NO:137) and contains PARP catalytic activity (Smith et al., supra). Similarly, the putative catalytic domain of TANK2-LONG (aa 1242-1382 of SEQ ID NO:133) and TANK2-SHORT (aa 1023-1161 of SEQ ID NO:135) is highly homologous to the catalytic domain of TANK1 (130 of 139 amino acids are the same; 94% identity).

The central domain of TANK1 contains 24 ankyrin repeats, indicating that TANK1 might belong to the ankyrin family of proteins that bridge integral membrane proteins to the cytoskeleton [Bennett, J Biol Chem 267: 8703-6 (1992)]. The ankyrin repeat domain of TANK1 (aa 181-1110 of SEQ ID NO:4) is significantly homologous to a central domain of TANK2-LONG (aa 242-1078 of SEQ ID NO:133) and TANK2-SHORT (aa 23-859 of SEQ ID NO:135) (692 of 837 amino acids are the same; 83% identity).

Within the ankyrin repeat domain of TANK1 is a binding site for the telomeric repeat binding factor-1 (TRF1) (Smith et al., supra) that functions to regulate the length of telomeres [van Steensel and de Lange, Nature 385:740-3 (1997)]. The TRF1 binding domain of TANK1 (aa 436-797 of SEQ ID NO:4) is significantly homologous to a region of TANK2-LONG (aa 497-858 of SEQ ID NO:133) and TANK2-SHORT (aa 278-639 of SEQ ID NO:135) (297 of 364 amino acids are the same; 82% identity).

TANK1 also contains a sterile alpha module (SAM) domain [Smith et al., supra] that is thought to be involved in protein-protein interactions [Ponting, Protein Sci 4: 1928-30 (1995); Schultz et al., Protein Sci 6: 249-53 (1997)]. A region of TANK2-LONG (aa 1089-1154 of SEQ ID NO:133) and TANK2-SHORT (aa 870-935 of SEQ ID NO:135) is homologous to the SAM domain of TANK1 (aa 1023-1088 of SEQ ID NO:4) (50 of 66 amino acids are the same; 76% identity).

A comparison of several putative functional domains of TANK2 (catalytic domain, ankyrin repeats, TRF-1 binding domain, and SAM domain) with TANK1 is discussed above. The additional amino terminal sequence contained in TANK2-LONG (all residues amino terminal to the ankyrin repeats, i.e., aa 1-241 of SEQ ID NO:133) allows for a comparison with the amino terminus of TANK1. The amino terminus of TANK1 contains homopolymeric runs of histidines, prolines, and serines (HPS domain, i.e., aa 1- 180 of SEQ ID NO:4) [Smith et al., supra]. The amino terminus of TANK2-LONG does not contain a HPS domain nor is it significantly homologous with the amino terminus of TANK1. The amino terminus of TANK2-LONG is also 61 amino acid residues longer than TANK1 and is composed of 48.1% non-polar residues, 32.4% polar residues, and 19.5% charged residues.

TANK2-SHORT is 219 amino acid residues shorter than TANK2-LONG and only contains 22 amino acid residues amino terminal to the ankyrin repeats. Interestingly, the Drosophila melanogaster tankyrase gene (GenBank® Accession No. AF132196; SEQ ID NO:138) encodes a putative protein designated dTANK (SEQ ID NO:139) that only contains 21 amino acid residues amino terminal to its ankyrin repeats. The amino terminal ends of TANK--SHORT and dTANK are not significantly homologous, although the two proteins do share homology in the other putative functional domains discussed above. The catalytic domain of TANK2-SHORT (aa 1023-1161 of SEQ ID NO:135) is homologous to a region of dTANK (aa 1033-1171 of SEQ ID NO:139) (113 of 139 amino acids are the same; 81% identity). The putative ankyrin repeat domain of TANK2-SHORT (aa 23-859 of SEQ ID NO:135) is significantly homologous to a central domain of dTANK (aa 22-875 SEQ ID NO:139) (545 of 858 amino acids are the same; 64% identity). The putative TRF1 binding domain of TANK2-SHORT (aa 278-639 of SEQ ID NO:135) is significantly homologous to a region of dTANK (aa 277-633 SEQ ID NO:139) (241 of 364 amino acids are the same; 66% identity). The putative SAM domain of TANK2-SHORT (aa 870-935 of SEQ ID NO:135) is significantly homologous to a region of dTANK (aa 886-951 of SEQ ID NO:139) (31 of 66 amino acids are the same; 66% identity).

### EXAMPLE 3

### Preparation of Antibodies Immunoreactive with TANK2 Polypeptides

The present invention provides for antibodies with specificity for TANK2 polypeptides. Antibodies to TANK2 may be produced by any method known in the art typically including, for example, the immunization of laboratory animals with preparations of purified native TANK2, purified recombinant TANK2, purified recombinant fragments of TANK2, or synthetic peptides derived from the TANK2 predicted amino acid sequence. To maximize the probability of obtaining antibodies with appropriate specificity for TANK2, regions of the polypeptide may be selected for use as an immunogen based upon differences in those regions between TANK1 and TANK2. For example, alignment of TANK1 and TANK2 demonstrates that a region consisting of aa 969-974 of TANK1 (SEQ ID NO:4) is substantially different from the corresponding region (aa 1030-1042) of TANK2-LONG (SEQ ID NO:133). In addition, the amino terminal domains of TANK1 (aa 1-180 of SEQ ID NO:4) and TANK2-LONG (aa 1-241 of SEQ ID NO:133) are substantially different, as discussed above. These regions can be expressed as truncated polypeptides in an appropriate expression system for use as immunogen or to test polyclonal or monoclonal antibody preparations. Similar approaches can be applied to other regions of the TANK2 polypeptide. Likewise, synthetic peptides can be made to correspond to various regions of differences and such peptides can be utilized to generate specific polyclonal or monoclonal antibodies by methods known in the art. For examples, see discussions in Harlow et al. (1988), supra.

Alignment of TANK1 and TANK2 indicated that a region of TANK2-LONG consisting of aa 1030-1042 (SEQ ID NO:133) was substantially different than the corresponding region of TANK1 (aa 969-974 of SEQ ID NO:4). A peptide, designated ICEC #2, having this TANK2 sequence, was synthesized by AnaSpec Inc. (San Jose, Calif.) for use as an immunogen in antibody development. Peptide ICEC #2 was conjugated to KLH using Imjecte Maleimide Activated Carrier Proteins (Pierce, #77106) following the manufacturer's protocol.

Each of four 6 to 12 week old Balb/c mice were pre-bled on day 0 and immunized by subcutaneous injection of 50 μg per mouse of KLH-ICEC-2 peptide in Freund's complete adjuvant. Subsequent boosts were made on day 21 and 42 in Freund's incomplete adjuvant. Mice were test bled on day 52 and the bleeds were screened by ELISA, using standard methods, on plates coated with KLH-ICEC-2 peptide. Specific antibody was detected using goat anti-mouse IgG(fc) horseradish peroxidase (HRP) conjugate. Mouse #3616 was given pre-fusion boosts on day 118 and 119 with 50 μg KLH-ICEC-2 peptide in PBS. The spleen was removed and fused on day 122.

Splenocytes were fused to NS-1 cells in a ratio of 5:1 by standard methods using polyethylene glycol 1500 (Boehringer Mannheim/Roche Molecular Biochemicals) [Harlow et al. (1988), supra]. The fused cells were resuspended in 250 mL RPMI containing 15% FBS, 100 mM sodium hypoxanthine, 0.4 mM aminopterin, 16 mM thymidine (HAT) (Gibco BRL, Rockville, Md.), 10 units/mL IL-6 (Boehringer Mannheim/Roche Molecular Biochemicals) and 1.5×10⁶ murine thymocytes/mL. The suspension was dispensed into twelve and a half 96-well flat bottom tissue culture plates (Corning, United Kingdom) at 200 μL/well. Cells in plates were fed on days 4, 5, and 6 post fusion by aspirating approximately 100 μL from each xxell and adding 100 μL/well plating medium described above except lacking thymocytes.

Supernatants from the fused cells were screened on day 7-12, initially by ELISA on the immunogen, as described above. To ensure clonality, positive wells chosen from the fusion were subcdoned 3 times by limiting dilution, using media lacking aminopterin. Cloning was completed for one fusion, 345C, which remained reactive to the immunizing protein. Isotyping of the antibody was performed by standard ELISA methods, using goat anti-mouse IgG 1, IgG2a, IgG2b, and IgG3 HRP conjugates as detecting antibodies. The clone 345C was IgG1.

Western analysis was also used to test immunoreactivity of 345C to TANK2. 1×10⁷ non-proliferating human PBL cells were pelleted by centrifugation and lysed by addition of 0.5 mL Buffer D [0.1% NP 40, 0.1% TX-100, 100 mM KCl, 20 mM HEPES, pH 7.9, 0.2 mM EDTA, 0.2 mM EGTA, 1.0 mM dithiothreitol (DTT), and protease inhibitor cocktail tablets, (Boehringer Mannheim/Roche Molecular Biochemicals)]. Lysates were sonicated (Sonifier® 250, Branson Ultrasonics Corp., Danbury, Conn.) at 20% output for 30 seconds and clarified in a 4° C. microfuge for 5 min and the pellets discarded. Mouse IgG (2.5 μg) or 0.5 mL 345C mAb culture supernatant was added to the lysates and they were incubated for 90 min at 4° C. Immune complexes were collected by precipitation with 30 μL protein G-Agarose slurry (Pierce) with gentle rocking for 30 minutes at 4° C. Pellets were washed 4X in Buffer D, resuspended in 25 μL 1×SDS Sample buffer [50 mM Tris-HCl, pH 6.8, 2% SDS, 0.1% bromophenol blue, 10% glycerol, and 100 mM DDT], and heated for 5 min at 100° C.

Samples were electrophoresed on 8% Tris-Glycine polyacrylamide gels (Novex, San Diego, Calif.) at 60 mA for 30 min, as described by the manufacturer. Gels were transferred to Immobilon-P transfer membrane (Millipore, Bedford, Mass.) using a Bio-Rad (Hercules, Calif.) semi-dry blotting apparatus at 150 mA for 90 min as described by the manufacturer. Blots were then blocked in TBST buffer (Tris buffered saline, pH 7.5 and 0.5% Tween®) containing 5.0% nonfat dry milk for 20-30 min at room temperature. Primary mAb 345C culture supernatant was then added at a 1:2 dilution to TBST containing 1.0% nonfat dry milk and blots were incubated at room temperature for 90 min. Following 4 washes with TBST, secondary antibody (goat anti-mouse IgG HRP conjugate, Bio-Rad) was added at a 1/3,000 dilution in TBST containing 1.0% nonfat dry milk and blots were incubated for 30 min at room temperature. Blots were again washed 4× in TBST followed by incubation in ECL detection reagents (Amersham Life Sciences, Uppsala, Sweden) as described by the manufacturer, followed by exposure to X-ray film. Positive signals of approximately the expected size for TANK2-LONG and TANK2-SHORT were obtained. The entire procedure is repeated to obtain more strongly immunoreactive monoclonal antibodies.

### EXAMPLE4

### Analysis of Tank2 Expression bv Northern Blot Hybridization

In order to identify cell and tissue types that express tankyrase2 mRNA, Northern blot analysis was performed using commercially prepared multi-tissue Northern blots (Clontech). The DNA probe template was amplified by PCR using a primer (5-Tank2-15; SEQ ID NO:140) corresponding to the sense strand of FB2B.1 polynucleotide sequence (nt 2330-2349 of SEQ ID NO:88) and a primer (3-Tank2-18; SEQ ID NO:141) corresponding to the antisense strand of FB2B.1 polynucleotide sequence (nt 2656-2675 of SEQ ID NO:88).

| | | |
|---|---|---|
| 5-Tank2-15 | GGCCTGAAGGTATGGTCGAT | (SEQ ID NO:140) |
| 3-Tank2-18 | TGAGGGCATTACAGTTTGTT | (SEQ ID NO:141) |

The PCR reaction contained 100 ng FB2B.1 cDNA, 0.25 μM each primer, 0.20 mM dNTPs, 1×PCR buffer, and 1 μL of Clontech Advantage® polymerase mix. The reactions were performed in a GeneAmp® PCR System 9700 with the following steps: 1) 1 cycle at 94° C. for 1 min; 2) 30 cycles of 94° C. for 30 sec, 60° C. for 30 sec, and 72° C. for 30 sec; and 3) 1 cycle at 72° C. for 7 min. The PCR fragment (designated Tank2-Nprobe; SEQ ID NO:142) was isolated using gel electrophoresis and a QIAquick® kit as directed. Tank2-Nprobe was labeled with ³²P with a Random Primed DNA Labeling Kit (Boehringer Mannheim/Roche Molecular Biochemicals) as directed and used to probe Clontech multi-tissue Northern blots. Prehybridization with Clontech's ExpressHyb™ DNA Hybridization solution was performed at 68° C. for 30 min. Hybridization with labeled probe was performed for 1 hr at 68° C. in ExpressHyb™. The blots were washed three times at room temperature in buffer containing 2×SSC and 0.05% SDS and then washed two times at 50° C. in buffer containing 0.1×SSC and 0.1% SDS prior to autoradiography.

The tissue Northern blot contained an approximately 6.3 kb band whose signal was strongest in placenta, PBL, ovary, and spleen and was present in pancreas, kidney, skeletal muscle, liver, lung, brain, heart, colon, small intestine, testis, prostate, and thymus.

### EXAMPLE 5

### Analysis of Tank2 Expression by in situ Hybridization

Expression of tankyrase2 was examined in tissue sections by in situ hybridization as described below.

Preparation of probes

A probe for tankyrase2 in situ hybridization was generated using procedures routinely practiced in the art. A primer (5-Tank2-1 5p; SEQ ID NO:143) corresponding to the sense strand of FB2B. 1 polynucleotide sequence (nt 2330-2349 of SEQ ID NO:88) and a primer (3-Tank2-18p; SEQ ID NO:144) corresponding to the antisense strand of FB2B.1 polynucleotide sequence (nt 2656-2675 of SEQ ID NO:88) were synthesized for use in a PCR reaction using FB2B.1 as the template.

| | |
|---|---|
| 5-Tank2-15p | |
| GCCGAATTCGGCCTGAAGGTATGGTCGAT | (SEQ ID NO:143) |
| 3-Tank2-18p | |
| GCCGAATTCTAGATGAGGGCATTACAGTTTGTT | (SEQ ID NO:144) |

The PCR reaction contained 100 ng FB2B.1 cDNA, 0.5 μM each primer, 0.25 mM dNTPs, 1×PCR buffer, and 2.5 U of PfuTurbo® polymerase mix (Stratagene). The reactions were performed in a GeneAmp® PCR System 9700 with the following steps: 1) 1 cycle at 94° C. for 1 min; 2) 25 cycles of 94° C. for 30 sec, 55° C. for 1 min, and 72° C. for 1 min; and 3) 1 cycle at 72° C. for 7 min. The PCR fragment was digested with EcoRI, isolated using gel electrophoresis and a QlAquick® kit, and subcloned into a Bluescript® vector (Stratagene). The clone, designated Tank2-ISprobe, was sequenced with the M13 primers designed to anneal to the vector (SEQ ID NOs:25 and 26) and the sequence is set out in SEQ ID NO:145. Tank2-ISprobe was digested with XhoI and transcribed (see below) with T3 polymerase to generate an antisense probe. A sense probe was generated by digesting Tank2-ISprobe with BamHI and transcribing with T7 polymerase.

To compare the tissue expression of tankyrase2 with tankyrase1, a tankyrase1 probe was generated. The tankyrase1 probe corresponds to a region in the 3′ untranslated sequence of the tankyrase1 gene. The 3′ untranslated sequence of tankyrase1, designated 3-Tank1UT, is set out in SEQ ID NO:146. A primer (5-Tank1-7p; SEQ ID NO:147) corresponding to the sense strand of 3-Tank1UT polynucleotide sequence (nt 407-426 of SEQ ID NO:146) and a primer (3-Tank1-13p; SEQ ID NO:148) corresponding to the antisense strand of 3-Tank1 UT polynucleotide sequence (nt 742-767 of SEQ ID NO:146) were synthesized for use in a PCR reaction using 3-Tank1UT as the template.

| | |
|---|---|
| 5-Tank1-7p | |
| GCCGAATTCCTTGTTTTTGATTTGCCAGA | (SEQ ID NO:147) |
| 3-Tank1-13p | |
| GCCGAATTCCGGCTTTGACTTCTCTGAATTTAGG | (SEQ ID NO:148) |

The PCR reaction contained 100 ng 3-Tank1UT cDNA, 0.5 μM each primer, 0.25 mM dNTPs, 1×PCR buffer, and 2.5 U of PfuTurbo® polymerase mix (Stratagene). The reactions were performed in a GeneAmp® PCR System 9700 with the following steps: 1) 1 cycle at 94° C. for 1 min; 2) 30 cycles of 94° C. for 30 sec, 55° C. for 1 min, and 72° C. for 1 min; and 3) 1 cycle at 72° C. for 7 min. The PCR fragment was digested with EcoRl, isolated using gel electrophoresis and a QIAquick® kit, and subcloned into a Bluescript® vector (Stratagene). The clone, designated Tank1-ISprobe, was sequenced with the M13 primers (SEQ ID NOs:25 and 26) and the sequence is set out in SEQ ID NO:149. Tank1-ISprobe was digested with BamHI and transcribed with T7 polymerase to generate an antisense probe. A sense probe was generated by digesting Tank1-ISprobe with Ahol and transcribing with T3 polymerase.

The Tank1-IS probe and Tank2-ISprobe were transcribed using a RNA Transcription kit (Stratagene) in a reaction containing 5 μL of 5×transcription buffer, 30 mM DTT, 0.8 mM each ATP CTP, GTP, 40 U RNase Block II, 12.5 U T3 or T7 polymerase, 300 ng linearized plasmid template, and 50 μCi ³⁵S-UTP (greater than 1000 Ci/mmol, Amersham, Arlington Heights, Ill.). The mixture was incubated at 37° C. for 1 hr, after which the template DNA was removed by addition of 1 μL of RNase-free DNase I (Stratagene) and incubated for 15 min at 37° C. A Quick Spin G50 RNA column (5′→3′ Inc., Boulder, Colo.) was prepared according to the manufacturer's suggested protocol. Twenty-five microliters (25 μL) of dH₂O was added to the probe and it was placed in the center of the column and the column centrifuged for 4 min at 1100 rpm in a desk top centrifuge. The column flow-through was mixed with 50 μL dH₂O, 2 μL of a 10 mg/mL tRNA solution, 10 μL 3 M sodium acetate, and 200 μL 100% ethanol (VWR, So. Plainfield, N.J.) and the resulting mixture was incubated at −20° C. overnight. The probe solution was centrifuged for 15 min at 4° C., the supernatant was removed, and the pellet was resuspended in 40 μL 1×TBE [90 mM Tris-Borate and 2 mM EDTA (pH 8.0)] containing 1 μL of 0.1 M DTT. The probe was stored at −70° C. until the in situ hybridization was performed.

Preparation of tissue samples and in situ hybridization

Tissues (National Disease Research Interchange, Philadelphia, Pa. and Cooperative Human Tissue Network, Philadelphia, Pa.) were sectioned at 6 μm and placed on Superfrost® Plus slides (VWR). Sections were fixed for 20 min at 4° C. in 4% paraformaldehyde (Sigma, St. Louis, Mo.). The slides were rinsed in three changes of 1×CMF-PBS, dehydrated with three successive washes with 70% ethanol, 95% ethanol, and 100% ethanol, and dried for 30 min at room temperature. The slides were placed in 70% formamide (J. T. Baker, Phillpsburg, N.J.) in 2×SSC for 2 min at 70° C., rinsed in 2×SSC at 4° C., dehydrated through 70%, 95%, and 100% ethanol washes, and dried for 30 min at room temperature. Slides were placed in an airtight box containing a piece of filter paper saturated with box buffer containing 50% formamide in 4×SSC. The probes, as described above, were individually prepared by mixing 4×10⁵ cpm/ tissue section with 5 μL of a 10 mg/mL tRNA solution per section and heating the mixture at 95° C. for 3 min. Ice-cold rHB2 buffer [10% dextran sulfate (Sigma), 50% formamide, 100 mM DTT (Boehringer Mannheim/Roche Molecular Biochemicals), 0.3 M NaCl (Sigma), 20 mM Tris, pH 7.5, 5 mM EDTA (Sigma), and 1×Denhardt's solution (Sigma)] was added to the probe mixture to bring the final volume to 60 μL/section. The probe solution was then added to the tissue sections. The slides were incubated at 50° C. for 12-16 hr. Following hybridization, the slides were washed once in 4×SSC containing 10 mM DTT for 1 hr at room temperature, once in 50% deionized formamide, 1×SSC, and 1 mM DTT for 40 min at 60° C., once in 2×SSC for 30 min at room temperature, and once in 0.1×SSC for 30 min at room temperature. The sections were dehydrated through 70%, 95%, and 100% ethanol washes and air dried for 30 min. The slides were dipped in Kodak (Rochester, N.Y.) NTB2 nuclear emulsion at 45° C. for 3 hr at room temperature in the dark and stored in the dark at 4° C. with desiccant until time of development.

The slides were rinsed in dH₂O and stained with hematoxylin and eosin by transfer of the slides through a series of the following steps: 5 min in forrnaldehyde/alcohol (100 mL formaldehyde, 900 mL 80% ethanol); three rinses in water for a total of 2 min; 5 min in 0.75% Harris hematoxylin (Sigma); three rinses in water for a total of 2 min; one dip in 1% HCl/50% ethanol; one rinse in water; four dips in 1% lithium carbonate; 10 min in tap water; 2 min in 0.5% eosin (Sigma); three rinses in water for a total of 2 min; 2 min in 70% ethanol; three 1 min rinses in 95% ethanol; two 1 min rinses in 100% ethanol; and two 2 min rinses in xylene. Slides were mounted with cytoseal 60 (Stephens Scientific, Riverdale, N.J.).

The signals obtained with the antisense tankyrase1 or antisense tankyrase2 probes were compared to the control signals obtained by the respective sense probes and any signal specific to the antisense tankyrase1 or antisense tankyrase2 probe was assumed to represent tankyrase1 or tankyrase2 expression, respectively. Both tankyrase1 and tanyrase2 signal was detected in most areas of the human testis, including the spermatogonia and spermatocytes. Tankyrase1 signal was detected in the red pulp of the human spleen while tankyrase2 signal was detected in the white pulp of the human spleen. The probes for tankyrase1 and tankyrase2 are used to detect expression in other tissues in a similar manner. Tankyrase1 signal was detected uniformly in mouse embryo, with the highest signal present in the skin. Tankyrase2 signal was also detected uniformly in mouse embryo, with the highest signal present in the mesenchymal areas and in the brain.

### EXAMPLE 6

### Identification of a Tankyrase2 Binding Partner

As described above, TANK1 interacts with the telomere-specific DNA binding protein TRF1 [Smith et al., (1998), supra]. The polynucleotide sequence of TRF1 is set out in SEQ ID NO:150, and the amino acid sequence of TRF1 is set out in SEQ ID NO:151. The yeast two-hybrid system [Hollenburg et al., Mol Cell Biol 15:3813-22 (1995)] was used to determine if TANK2 also interacts with TRF1. In this yeast two-hybrid system, the yeast strain L40 has been engineered to contain multiple LexA binding sites upstream of the HIS3 and beta-galactosidase genes. Interaction of one protein fused to LexA (created in the BTM116 vector) with a second protein fused to the VP 16 activation domain (created in the VP16 vector) results in the expression of HIS3, allowing yeast growth in media lacking histidine. Interaction of the two proteins also results in the expression of the beta-galactosidase gene, which can be measured in a colorometric assay [Breeden and Nasmyth, Cold Spring Harbor Symp Quant Biol 643-650 (1985)]

The TANK1 binding domain of TRF1, here designated TRF1-TankBD, has been mapped to an amino terminal region of TRF1. TRF1-TankBD was amplified by PCR using a primer (5-TRF1; SEQ ID NO:152) corresponding to the sense strand of TRF1 polynucleotide sequence (nt 1-24 of SEQ ID NO:150) and a primer (3-TRF1; SEQ ID NO:153) corresponding to the antisense strand of TRF1 polynucleotide sequence (nt 184-201 of SEQ ID NO:150).

(SEQ ID NO:152)

| | |
|---|---|
| 5-TRF1 | GCCCCGGGGATCCTCATGGCGGAGGATGTTTCCTCAGCG |

(SEQ ID NO:153)

| | |
|---|---|
| 3-TRF1 | TCCCGGGGATCCTCACACCAGGCCCGCGTCCTC |

The PCR reaction contained 5 μL Clontech human testis Marathon®-Ready cDNA, 0.20 μM each primer, 0.20 mM dNTPs, 1×PCR buffer, and 1 μL of Clontech Advantage® polymerase mix. The reactions were performed in a GeneAmp® PCR System 9700 with the following steps: 1) 1 cycle at 94° C. for 1 min; 2) 30 cvcles of 94° C. for 30 sec, 60° C. for 30 sec. and 72° C. for 30 sec; and 3) 1 cycle at 72° C. for 7 min. The PCR fragment was digested with BamHI, isolated using gel electrophoresis and a QIAquick® kit as directed, and subcloned into the BTM116 vector. TRF1-TankBD was sequenced with the M13 reverse primer designed to anneal to the vector (SEQ ID NO:26) and a primer designed to anneal to the cDNA sequence (SEQ ID NO:153). The polynucleotide sequence of TRF1-TankBD is set out in SEQ ID NO:154 and the amino acid sequence is set out in SEQ ID NO:155.

As described above, the TRF1 binding domain of TANK1 is very homologous to a region of TANK2 comprised of aa 497-858 of SEQ ID NO:133. The polynucleotide region corresponding to this domain of TANK2, designated Tank2-TRF1BD, was amplified in a PCR reaction with a primer (5-T2/TRF1BD; SEQ ID NO:156) corresponding to the sense strand of the tank2 polynucleotide sequence (nt 1717-1742 of SEQ ID NO:132) and a primer (3-T2/TRF1BD; SEQ ID NO:157) corresponding to the antisense strand of the tank2 polynucleotide sequence (nt 2765-2805 of SEQ ID NO:132).

| | | |
|---|---|---|
| 5-T2/TRF1BD | CGCAGGATCCCCTTCACTCCTCTTCATGAGGCAGCTTC | (SEQ ID NO:156) |
| 3-T2/TRF1BD | GGATCCGCTAAATATCTGTATCTCCATCTTTAACAAGATCCAAAGGAG | (SEQ ID NO:157) |

The PCR reaction contained 5 μL Clontech human testis Marathon®-Ready cDNA, 0.5 μM each primer, 0.25 mM dNTPs, 1×PCR buffer, and 2.5 U of PfuTurbo® polymerase mix (Stratagene). The reactions were performed in a GeneAmp® PCR System 9700 with the following steps: 1) 1 cycle at 94° C. for 1 min; 2) 30 cycles of 94° C. for 30 sec, 55° C. for 2 min, and 72° C. for 2 min; and 3) 1 cycle at 72° C. for 7 min. The PCR fragment was isolated using gel electrophoresis and a QIAquick® kit as directed, and subcloned into the pCR-BluntII™-TOPO® vector (Invitrogen). Tank2-TRFIBD was digested from the pCR-BluntII™-TOPO® with BamHI, and subcloned into the VP16 vector. The Tank2-TRF1BD clone was sequenced with primers designed to adhere to the vector sequence: M13 forward (SEQ ID NO:25) and 009 (SEQ ID NO:158).

009 GCCGACTTCGAGTTTGAGCAG (SEQ ID NO:158)

The polynucleotide sequence is set out in SEQ ID NO:159 and the amino acid sequence is set out in SEQ ID NO:160.

Co-transformation of L40 with the TRF1 -TankBD and Tank2-TRF1BD plasmids indicated that like TANK1, TANK2 binds to TRF1.

### EXAMPLE 7

### Measurement of TANK2 Biological Activity Construction of Expression Plasmids

The primary structure of the tankyrase2 polypeptide suggests that TANK2, like TANK1, will have poly(ADP-ribose) polymerase activity. The PARP activity of TANK2, or some substructure thereof, can be measured by the ability of that component to incorporate the ADP-ribose unit from AND into polymers of ADP-ribose coupled to a protein substrate. For example, TANK1 adds polymers of ADP-ribose to the TRF-1 protein in molecular assays [Smith et al., supra]. TANK2 is expected to also perform this function and/or to ADP-ribosylate another substrate or substrates. The demonstration of such activity on a given substrate is readily accomplished by the skilled artisan [see, for example, Smith et al., supra].

Structural differences in TANK1 and TANK2 suggest the possibility that TANK2 may have different protein substrate specificity than does TANK1. As demonstrated by the observation that TANK1 binds to TRF-1 and poly ADP-ribosylates TRF-1, it is anticipated that protein substrates of TANK2 can be identified by their ability to bind to TANK2. Additional substrates that bind TANK2 can be identified by a number of methods as described elsewhere in this application.

A fusion protein, designated PARP1A/TANK2B, containing aa 1-662 of PARP1 (SEQ ID NO:137) fused upstream of aa 996-1385 of TANK2 (SEQ ID NO:133) was used in the measurement of TANK2 poly(ADP-ribose) polymerase activity. PARP1A/TANK2B contained the DNA binding domain (aa 1-373 of SEQ ID NO:137) and automodification domain (aa 373-525 of SEQ ID NO:137) of PARP1 and the putative catalytic domain of TANK2 (aa 1242-1382 of SEQ ID NO:133).

The PARP1A piece of the fusion protein was amplified by PCR using a primer (Sal-PARP1; SEQ ID NO:161) corresponding to the sense strand of parp1 polynucleotide sequence (nt 1-30 of SEQ ID NO:136) and a primer (revMlu-PARP1; SEQ ID NO:162) corresponding to the antisense strand of parp1 polynucleotide sequence (nt 1957-1985 of SEQ ID NO:136).

| | | |
|---|---|---|
| Sal-PARP1 | CGTCGACCCATGGCGGAGTCTTCGGATAAGCTCTATCGA | (SEQ ID NO:161) |
| revMlu-PARP1 | GGAAACGCGTTTGGTGCCAGGATTTACTGTCAGCTTCTT | (SEQ ID NO:162) |

The PCR reaction contained 0.5 μL of human thymus and testis QUICK-Clone™ cDNA (Clontech), 0.25 μM each primer, 0.20 mM dNTPs, 1×PCR buffer, and 1 μL of Clontech Advantage® polymerase mix. The reactions were performed in a GeneAmp® (PE Applied Biosystems) with the following steps: 1) 1 cycle at 94° C. for 1 min; 2) 30 cycles of 94° C. for 30 sec, 60° C. for 2 min, and 72° C. for 2 min; and 3) 1 cycle at 72° C. for 7 min. The PCR fragment (designated parp1A) was isolated using gel electrophoresis and a QLAquick® kit as directed. Parp1A was subcloned into the pTrcHis2™-TOPO® vector (Invitrogen) as directed. Parp1A was digested from pTrcHis2™-TOPO® with SalI and MluI, the fragment isolated using gel electrophoresis and a QIAquick® kit, and saved for further subcloning described below.

The TANK2B piece of the fusion protein was amplified by PCR using a primer (forMlu-TANK2; SEQ ID NO:163) corresponding to the sense strand of tank2 polynucleotide sequence (nt 3214-3240 of SEQ ID NO:132) and a primer (TANK2-Strep-Not; SEQ ID NO:164) corresponding to the antisense strand of tank2 polynucleotide sequence (nt 4350-4383 of SEQ ID NO:132). ForMlu-TANK2

| | | |
|---|---|---|
| ForMlu-TANK2 | CTTAAACGCGTTGAAGGACAAACACCTTTAGATTTAGTT | (SEQ ID NO:163) |
| TANK2-Strep-Not | GTCGAAAGCGGCCGCTTAGCCTCCGAACTGTGGATGCC | (SEQ ID NO:164) |
| | TCCACGCTCCATCGACCATACCTTCAGGCCTCATAATCTGG | |

The PCR reaction contained 100 ng 2B.1 cDNA, 0.25 μM each primer, 0.20 mM dNTPs, 1×PCR buffer, and 1 μL of Clontech Advantage® polymerase mix. The reactions were performed in a GeneAmp® PCR System 9700 with the following steps: 1) 1 cycle at 94° C. for 1 min; 2) 30 cycles of 94° C. for 30 sec, 60° C. for 2 min, and 72° C. for 2 min; and 3) 1 cycle at 72° C. for 7 min. The PCR fragment (designated tank2B) was isolated using gel electrophoresis and a Q1Aquick® kit as directed. Tank2B was subcloned into the pCDNA3.1/NT-GFP-TOPO® vector (Invitrogen) as directed. Tank2B was digested from pCDNA3.1/NT-GFP-TOPO® with MluI and NotI and subcloned with SalI/MluI digested parp1A (see above) into a pFASTBAC vector (Gibco BRL), which had previously been digested with SalI and NotI. The resultant plasmid was designated pFB-PARP1A/TANK2B.

pFB-PARP1A/TANK2B was sequenced with primers designed to anneal to the vector sequence (SEQ ID NOs:165-166) and primers designed to anneal to the cDNA sequence (SEQ ID NOs:55, 60, and 66, supra, and SEQ ID NOs:167-176).

Vector Primers

Vector Primers

| | | |
|---|---|---|
| FastBac for | TTTGTTCGCCCAGACTC | (SEQ ID NO:165) |
| FastBac rev | TATGTTTCAGGTTCAGGGGGAG | (SEQ ID NO:166) |

cDNA Primers

| | | |
|---|---|---|
| P1 | GCGGAAGCTGGAGGAGTGAC | (SEQ ID NO:167) |
| P2 | GTCACTCCTCCAGCTTCCGC | (SEQ ID NO:168) |
| P3 | AAGCCCTGAAGAAGCAGCTC | (SEQ ID NO:169) |
| P4 | GAGCTGCTTCTTCAGGGCTT | (SEQ ID NO:170) |
| P5 | CAGACACCCAACCGGAAGGA | (SEQ ID NO:171) |
| P6 | TCCTTCCGGTTGGGTGTCTG | (SEQ ID NO:172) |
| P7 | TCCGCCTCCACCAAGAGCCT | (SEQ ID NO:173) |
| P8 | AGGCTCTTGGTGGAGGCGGA | (SEQ ID NO:174) |
| P9 | TGGCCTGGTGGACATCGTTA | (SEQ ID NO:175) |
| P10 | TAACGATGTCCACCAGGCCA | (SEQ ID NO:176) |

The nucleotide sequence of PARP1A/TANK2B is set out in SEQ ID NO:177 and the amino acid sequence of PARP1A/TANK2B is set out in SEQ ID NO:178. PARP1A/TANK2B consists of the following regions: a HIS tag leader region at aa 1-36; a PARP1 region at aa 37-698; a spacer region at aa 699-700; a TANK2 region at aa 701-1090; and a Strep-tag region at aa 1091-1099.

Production of Recombinant Viral Stocks and Protein Purification

PARP1A/TANK2B recombinant viral stock was produced using the FastBac system (Gibco BRL) according to the manufacturer's suggested protocol and protein expression was carried out as follows. Sf9 cells were grown at 27° C. in CCM3 medium (Hyclone, Logan, Utah) containing 50 U/mL penicillin and 50 μg/mL streptomycin sulfate (Gibco BRL). Exponentially growing cells were infected at a multiplicity of infection of approximately 0.5 virus per cell and incubated for 48 hr. Cells were collected by centrifugation at 1000×g for 15 min, and the pellets were frozen and stored at −80° C. until use.

For protein purification, reagents were obtained from Sigma unless otherwise indicated. Cells were lysed in Lysis buffer [25 mM Tris-HCl, pH 9.0, 50 mM glucose, 10 mM EDTA, 1 mM 2-mercaptoethanol, 1 mM PMSF, 100 μM antipain, and 2 μg/mL aprotinin] by sonication. Igepal CA-630 (final concentration of 0.2%), Tween®-20 (final concentration of 0.2%), and NaCl (final concentration of 0.5 M) were added to the Lysis buffer and the samples were agitated for 30 min at 4° C. The supernatants were collected after centrifugation at 20,000×g for 20 min at 4° C., at which time they were treated with 1 mg/mL protamine sulfate and allowed to stir for 1 hr at 4° C. The supernatants were collected after centrifugation at 4,000×g for 20 min at 4° C. at which time the protein was precipitated with 70% ammonium sulfate. Protein pellets were collected by centrifugation at 20,000×g for 15 min at 4° C. and resuspended in Re-suspension buffer [100 mM Tris-HCl, pH 7.4, 0.5 mM EDTA, 10% glycerol, 1 mM PMSF, and 12 mM 2-mercaptoethanol].

Proteins were first purified via the HIS tag using Talon® Superflow metal affinity resin (Clontech) and eluted with 200 mM imidazole (Clontech) as directed. The protein elutions were next purified using a 3-aminobenzamide Affi-Gel® matrix (Bio-Rad Laboratories) prepared as described elsewhere [D'Amours et al., Anal Biochem 249:106-8 (1997)]. Proteins were eluted with 10 mM 3-methoxybenzamide in Elution buffer [50 mM Tris-HCl, pH 7.5, 0.3 M NaCl, 10 mM 2-mercaptoethanol, 1 mM PMSF, 100 μM antipain, and 2 μg/mL aprotinin]. The proteins were dialyzed 4×in 1 L Dialysis buffer [50 mM Tris-HCl, pH 8.0, 1 mM DTT, 4 mM MgCl₂, 10 mM EDTA, 1 mM PMSF, and 2 μg/mL aprotinin). Glycerol was added to a final concentration of 10% and the proteins were stored at −80° C.

Poly(ADP-ribose) polymerase activity

For poly(ADP-ribose) polymerase activity assays, reagents were obtained from Sigma unless otherwise indicated. PARP1A/TANK2B (250 ng) protein was incubated for 10 min at room temperature in Assay buffer (total volume of 20 μL) [100 mM Tris-HCl, pH 8.0, 10 mM MgCl₂, 10% glycerol, 1.5 mM DTT (Boehringer Mannheim/Roche Molecular Biochemicals), 2.5 μM unlabeled NAD⁺, 16.7 μg/mL E. coli Strain B DNA, and 0.33 μCi γ-[³²P]-NAD⁺ (NEN, Boston, Mass.). Reactions were stopped by boiling in SDS running buffer and separated by SDS-polyacrylamide gel electrophoresis (SDS-PAGE). Autoradiography was used to visualize labeled protein. Addition of poly(ADP-ribose) polymers to protein substrate results in an increase in molecular weight of the protein, and consequently causes the protein to run higher on SDS PAGE. Also, the level of poly(ADP-ribose) polymers added to the protein substrate can vary with each single protein molecule, resulting in labeled proteins with different molecular weights, which appears on the autoradiography film as a ladder or smear [for example, see Smith et al. Science 282:2484-7 (1998)]. PARP1A/TANK2B possessed intrinsic poly(ADP-ribose) polymerase activity as shown by its ability produce poly(ADP-ribose) polymers. The PARP1A/TANK2B poly(ADP-ribose) polymerase reaction produced a ladder of labeled protein from approximately 136 kDa to 250 kDa.

All publications and patent documents cited in this specification are incorporated herein by reference for all that they disclose.

While the present invention has been described with specific reference to certain preferred embodiments for purposes of clarity and understanding, it will be apparent to the skilled artisan that further changes and modifications may be practiced within the scope of the invention as it is defined in the claims set forth below. Accordingly, no limitations should be placed on the invention other than those specifically recited in the claims.

What is claimed is:

1. A purified and isolated tankyrase2 polypeptide.

2. The polypeptide according to claim 1, comprising the amino acid sequence defined in SEQ ID NO:133.

3. The polypeptide according to claim 1, comprising the amino acid sequence defined in SEQ ID NO:135.

4. A polynucleotide encoding the polypeptide according to claim 1.

5. The polynucleotide according to claim 4, comprising the coding region of the nucleotide sequence defined in SEQ ID NO:132.

6. The polynucleotide according to claim 4, comprising the coding region of the nucleotide sequence defined in SEQ ID NO:134.

7. A polynucleotide selected from the group consisting of:
(a) the polynucleotide according to claim 4,
(b) a polynucleotide complementary to the polynucleotide of (a), and
(c) a polynucleotide that hybridizes under moderately stringent hybridization conditions to the polynucleotide of (a) or (b).

8. The polynucleotide according to claim 7, wherein the polynucleotide is a DNA molecule or an RNA molecule.

9. The polynucleotide according to claim 8, further comprising a detectable label moiety.

10. An expression construct, comprising the polynucleotide according to claim 4.

11. A host cell transformed or transfected with the expression construct according to claim 10.

12. The polynucleotide according to claim 4, wherein the polynucleotide is operatively linked to a heterologous promoter.

13. A host cell, comprising the polynucleotide according to claim 12.

14. A method for producing a tankyrase2 polypeptide, comprising the steps of:
a) growing the host cell according to claim 11 or 13 under conditions appropriate for expression of the polypeptide; and
b) isolating the polypeptide from the host cell or the medium in which the host cell is grown.

15. An antibody that is specifically immunoreactive with the polypeptide according to claim 1.

16. The antibody according to claim 15, wherein the antibody is selected from the group consisting of monoclonal antibodies, polyclonal antibodies, single chain antibodies (scFv antibodies), chimeric antibodies, bifunctional/bispecific antibodies, humanized antibodies, human antibodies, CDR-grafted antibodies, Fab fragments, Fab′ fragments, F(ab′)₂ fragments, and Fv fragments.

17. A cell line that produces an antibody according to claim 15.

18. An anti-idiotype antibody that is specifically immunoreactive with an antibody according to claim 15.

19. A method for identifying a binding partner of a tankyrase2 polypeptide, comprising:
a) contacting the tankyrase2 polypeptide with a test compound under conditions that permit binding of the tankyrase2 polypeptide and the test compound;
b) detecting binding of the test compound and the tankyrase2 polypeptide; and
c) identifying the test compound as a binding partner of the tankyrase2 polypeptide.

20. The method according to claim 19, wherein said specific binding partner selectively or specifically modulates a biological activity of the tankyrase2 polypeptide.

21. A method for identifying a specific binding partner of a tankyrase2 polynucleotide, comprising:
a) contacting the tankyrase2 polynucleotide with a test compound under conditions that permit binding of the tankyrase2 polynucleotide and the test compound;
b) detecting binding of the test compound and the tankyrase2 polynucleotide; and
c) identifying the test compound as a specific binding partner of the tankyrase2 polynucleotide.

22. The method according to claim 21, wherein said binding partner selectively or specifically modulates activity of the tankyrase2 polynucleotide.

23. A method of treating an animal having a medical condition mediated by poly(ADP-ribose) polymerase activity, comprising administering to said animal a tankyrase2 inhibitory compound in an amount effective for inhibiting tankyrase2 activity in said animal.

24. The method according to claim 23, wherein said medical condition is associated with growth of neoplastic tissue.

25. The method according to claim 24, wherein said neoplastic tissue is a cancer selected from the group consisting of carcinomas, sarcomas, leukemias, and lymphomas.

26. The method according to claim 25, wherein said cancer is selected from the group consisting of ACTH-producing tumor, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and non-small cell), malignant peritoneal effusion, malignant pleural effusion, melanoma, mesothelioma, multiple myeloma, neuroblastoma, glioma, non-Hodgkin's lymphoma, osteosarcoma, ovarian cancer, ovarian (germ cell) cancer, pancreatic cancer, penile cancer, prostate cancer, retinoblastoma, skin cancer, soft tissue sarcoma, squamous cell carcinomas, stomach cancer, testicular cancer, thyroid cancer, trophoblastic neoplasms, uterine cancer, vaginal cancer, cancer of the vulva, and Wilm's tumor.


SEQUENCE LISTING (178 sequences)

SEQ ID NO: 1 - DNA, 3508 bp, Homo sapiens
  CDS (2)..(3508)
ggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggagctgatgtccatgctaaagataaaggtgatctggtaccattacacaatgcctgttcttatggtcattatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatgga

SEQ ID NO: 2 - protein, 1169 aa, Homo sapiens
ARIMSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGADVHAKDKGDLVPLHNACSYGHYEVTELLVKHGACVNAMDLWQFTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 3 - DNA, 3984 bp, Homo sapiens
  CDS (1)..(3981)
atggcggcgtcgcgtcgctctcagcatcatcaccaccatcatcaacaacagctccagcccgccccaggggcttcagcgccgccgccgccacctcctcccccactcagccctggcctggccccggggaccaccccagcctctcccacggccagcggcctggcccccttcgcctccccgcggcacggcctagcgctgccggagggggatggcagtcgggatccgcccgacaggccccgatccccggacccggttgacggtaccagctgttgcagtaccaccagcacaatctgtaccgtcgccgccgctcccgtggtcccagcggtttctacttcatctgccgctggggtcgctcccaacccagccggcagtggcagtaacaattcaccgtcgtcctcttcttccccgacttcttcctcatcttcctctccatcctcccctggatcgagcttggcggagagccccgaggcggccggagttagcagcacagcaccactggggcctggggcagcaggacctgggacaggggtcccagcagtgagcggggccctacgggaactgctggaggcctgtcgcaatggggacgtgtcccgggtaaagaggctggtggacgcggcaaacgtaaatgcaaaggacatggccggccggaagtcttctcccctgcacttcgctgcaggttttggaaggaaggatgttgtagaacacttactacagatgggtgctaatgtccacgctcgtgatgatggaggtctcatcccgcttcataatgcctgttcttttggccatgctgaggttgtgagtctgttattgtgccaaggagctgatccaaatgccagggataactggaactatacacctctgcatgaagctgctattaaagggaagatcgatgtgtgcattgtgctgctgcagcacggagctgacccaaacattcggaacactgatgggaaatcagccctggacctggcagatccttcagcaaaagctgtccttacaggtgaatacaagaaagacgaactcctagaagctgctaggagtggtaatgaagaaaaactaatggctttactgactcctctaaatgtgaattgccatgcaagtgatgggcgaaagtcgactcctttacatctagcagcgggctacaacagagttcgaatagttcagcttcttcttcagcatggtgctgatgttcatgcaaaagacaaaggtggacttgtgcctcttcataatgcatgttcatatggacattatgaagtcacagaactgctactaaagcatggagcttgtgttaatgccatggatctctggcagtttactccactgcacgaggctgcttccaagaaccgtgtagaagtctgctctttgttacttagccatggcgctgatcctacgttagtcaactgccatggcaaaagtgctgtggatatggctccaactccggagcttagggagagattgacttatgaatttaaaggtcattctttactacaagcagccagagaagcagacttagctaaagttaaaaaaacactcgctctggaaatcattaatttcaaacaaccgcagtctcatgaaacagcactgcactgtgctgtggcctctctgcatcccaaacgtaaacaagtgacagaattgttacttagaaaaggagcaaatgttaatgaaaaaaataaagatttcatgactcccctgcatgttgcagccgaaagagcccataatgatgtcatggaagttctgcataagcatggcgccaagatgaatgcactggacacccttggtcagactgctttgcatagagccgccctagcaggccacctgcagacctgccgcctcctgctgagttacggctctgacccctccatcatctccttacaaggcttcacagcagcacagatgggcaatgaagcagtgcagcagattctgagtgagagtacacctatacgtacttctgatgttgattatcgactcttagaggcatctaaagctggagacttggaaactgtgaagcaactttgcagctctcaaaatgtgaattgtagagacttagagggccggcattccacgcccttacacttcgcagcaggctacaaccgcgtgtctgttgtagagtacctgctacaccacggtgccgatgtccatgccaaagacaagggtggcttggtgccccttcataatgcctgttcatatggacactatgaggtggctgagcttttagtaaggcatggggcttctgtcaatgtggcggacttatggaaatttacccctctccatgaagcagcagctaaaggaaagtatgaaatctgcaagctccttttaaaacatggagcagatccaactaaaaagaacagagatggaaatacacctttggatttggtaaaggaaggagacacagatattcaggacttactgaaaggggatgctgctttgttggatgctgccaagaagggctgcctggcaagagtgcagaagctctgtaccccagagaatatcaactgcagagacacccagggcagaaattcaacccctctgcacctggcagcaggctataataacctggaagtagctgaatatcttctagagcatggagctgatgttaatgcccaggacaagggtggtttaattcctcttcataatgcggcatcttatgggcatgttgacatagcggctttattgataaaatacaacacgtgtgtaaatgcaacagataagtgggcgtttactcccctccatgaagcagcccagaaaggaaggacgcagctgtgcgccctcctcctagcgcatggtgcagaccccaccatgaagaaccaggaaggccagacgcctctggatctggcaacagctgacgatatcagagctttgctgatagatgccatgcccccagaggccttacctacctgttttaaacctcaggctactgtagtgagtgcctctctgatctcaccagcatccaccccctcctgcctctcggctgccagcagcatagacaacctcactggccctttagcagagttggccgtaggaggagcctccaatgcaggggatggcgccgcgggaacagaaaggaaggaaggagaagttgctggtcttgacatgaatatcagccaatttctaaaaagccttggccttgaacaccttcgggatatctttgaaacagaacagattacactagatgtgttggctgatatgggtcatgaagagttgaaagaaataggcatcaatgcatatgggcaccgccacaaattaatcaaaggagtagaaagactcttaggtggacaacaaggcaccaatccttatttgacttttcactgtgttaatcagggaacgattttgctggatcttgctccagaagataaagaatatcagtcagtggaagaagagatgcaaagtactattcgagaacacagagatggtggtaatgctggcggcatcttcaacagatacaatgtcattcgaattcaaaaagttgtcaacaagaagttgagggagcggttctgccaccgacagaaggaagtgtctgaggagaatcacaaccatcacaatgagcgcatgttgtttcatggttctcctttcattaatgccattattcataaagggtttgatgagcgacatgcatacataggaggaatgtttggggccgggatttattttgctgaaaactcctcaaaaagcaaccaatatgtttatggaattggaggaggaacaggctgccctacacacaaggacaggtcatgctatatatgtcacagacaaatgctcttctgtagagtgacccttgggaaatcctttctgcagtttagcaccatgaaaatggcccacgcgcctccagggcaccactcagtcattggtagaccgagcgtcaatgggctggcatatgctgaatatgtcatctacagaggagaacaggcatacccagagtatcttatcacttaccagatcatgaagccagaagccccttcccagaccgcaacagccgcagagcagaagacctag

SEQ ID NO: 4 - protein, 1327 aa, Homo sapiens
MAASRRSQHHHHHHQQQLQPAPGASAPPPPPPPPLSPGLAPGTTPASPTASGLAPFASPRHGLALPEGDGSRDPPDRPRSPDPVDGTSCCSTTSTICTVAAAPVVPAVSTSSAAGVAPNPAGSGSNNSPSSSSSPTSSSSSSPSSPGSSLAESPEAAGVSSTAPLGPGAAGPGTGVPAVSGALRELLEACRNGDVSRVKRLVDAANVNAKDMAGRKSSPLHFAAGFGRKDVVEHLLQMGANVHARDDGGLIPLHNACSFGHAEVVSLLLCQGADPNARDNWNYTPLHEAAIKGKIDVCIVLLQHGADPNIRNTDGKSALDLADPSAKAVLTGEYKKDELLEAARSGNEEKLMALLTPLNVNCHASDGRKSTPLHLAAGYNRVRIVQLLLQHGADVHAKDKGGLVPLHNACSYGHYEVTELLLKHGACVNAMDLWQFTPLHEAASKNRVEVCSLLLSHGADPTLVNCHGKSAVDMAPTPELRERLTYEFKGHSLLQAAREADLAKVKKTLALEIINFKQPQSHETALHCAVASLHPKRKQVTELLLRKGANVNEKNKDFMTPLHVAAERAHNDVMEVLHKHGAKMNALDTLGQTALHRAALAGHLQTCRLLLSYGSDPSIISLQGFTAAQMGNEAVQQILSESTPIRTSDVDYRLLEASKAGDLETVKQLCSSQNVNCRDLEGRHSTPLHFAAGYNRVSVVEYLLHHGADVHAKDKGGLVPLHNACSYGHYEVAELLVRHGASVNVADLWKFTPLHEAAAKGKYEICKLLLKHGADPTKKNRDGNTPLDLVKEGDTDIQDLLKGDAALLDAAKKGCLARVQKLCTPENINCRDTQGRNSTPLHLAAGYNNLEVAEYLLEHGADVNAQDKGGLIPLHNAASYGHVDIAALLIKYNTCVNATDKWAFTPLHEAAQKGRTQLCALLLAHGADPTMKNQEGQTPLDLATADDIRALLIDAMPPEALPTCFKPQATVVSASLISPASTPSCLSAASSIDNLTGPLAELAVGGASNAGDGAAGTERKEGEVAGLDMNISQFLKSLGLEHLRDIFETEQITLDVLADMGHEELKEIGINAYGHRHKLIKGVERLLGGQQGTNPYLTFHCVNQGTILLDLAPEDKEYQSVEEEMQSTIREHRDGGNAGGIFNRYNVIRIQKVVNKKLRERFCHRQKEVSEENHNHHNERMLFHGSPFINAIIHKGFDERHAYIGGMFGAGIYFAENSSKSNQYVYGIGGGTGCPTHKDRSCYICHRQMLFCRVTLGKSFLQFSTMKMAHAPPGHHSVIGRPSVNGLAYAEYVIYRGEQAYPEYLITYQIMKPEAPSQTATAAEQKT

SEQ ID NO: 5 - DNA, 460 bp, Homo sapiens
  misc_feature (136): n= a, c, g, or t
gaactgtcttcagtagttagttcaagtggaacagagggtgcttccagtttggagaaaaaggaggttccaggagtagattttagcataactcaattcgtaaggaatcttggacttgagcacctaatggatatatttnagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaaca

SEQ ID NO: 6 - protein, 42 aa, Homo sapiens
GTILIDLSPDDKEFQSVEEEMQSTVREHRDGGHAGGIFNRYN

SEQ ID NO: 7 - DNA, 564 bp, Homo sapiens
  misc_feature (203): n= a, c, g, or t
tgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggagnctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggtaatgtagttttatttgttcatcttcaaaantgctaggggaggcatactttaactttttattaatctcttgaattgacaagacntttgccttaacgggnttttttaaaattttatttgggggtattttcagtttgggaagttacaaatagtaaagagattttcttattacccttacccggnttccnaatgttatattttgttccctt

SEQ ID NO: 8 - protein, 118 aa, Homo sapiens
  SITE (67): Xaa= unknown
LFHGSPFVNAIIHKGFDERHAYIGGMFGAGIYFAENSSKSNQYVYGIGGGTGCPVHKDRSCYICHRXLLFCRVTLGKSFLQFSAMKMAHSPPGHHSVTGRPSVNGLALAEYVIYRGEQ

SEQ ID NO: 9 - DNA, 397 bp, Homo sapiens
aacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttggggaaagattgagttatcagatttaatttgccgatgggagcctttatctgtcataggaaatctttctca

SEQ ID NO: 10 - DNA, 343 bp, Homo sapiens
  misc_feature (255): n= a, c, g, or t
cttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagttttaacattctgacttgataaagctttaataatgtacagtgttttctaaatatttnctgttttttcagcactttaacagatgccattccagggtaaactggggttgtctgtactaaattataaacagggttaactggacccttt

SEQ ID NO: 11 - DNA, 334 bp, Homo sapiens
gcagttctaaaacagatttatattacaaagcaatgtaaataaatactgggctagtacaaaagctcttatttacagttttacaaatgaaattgtattcagtgtaaatgctgtgttttaaagaacacagtattgtattagtaaaattagttctgttgagggcattacagtttgttagaatcaatgcataacatataaaaggttcaagttaactctgtttataatttagtacagacaacccagtttaacctggaatggcatctgttaaagtgctgaaaaaacaggaaatatttagaaaacactgtacattattaaagctttatcaagtcagaatgtt

SEQ ID NO: 12 - DNA, 353 bp, Homo sapiens
cagcaaaggagtaaaacgtagaggccactgctgctttgatgattttaggttcagtggaattagtttcttaaaataactatttatccatcgaccataccttcaggcctcataatctggtaagtaattaaatactcaggataagcctgttctcctctgtaaataacatattcagctaatgctaggccatttacactgggcctaccagtgactgagtgatgacctggaggagaatgtgccattttcattgcactgaactgcaggaaagactttcccaaggttacccggcaaaagagcagctgcctgtggcaaatgaacaagatctgtctttgtgaactggacacccagtaccttct

SEQ ID NO: 13 - DNA, 436 bp, Homo sapiens
  misc_feature (334): n= a, c, g, or t
ttttttttgcagttctaaaacagatttatattacaaagcaatgtaaataaatactgggctagtacaaaagctcttatttacagttttacaaatgaaattgtattcagtgtaaatgctgtgttttaaagaacacagtattgtattagtaaaattagttctgttgagggcattacagtttgttagaatcaatgcataacatataaaaggttcaagttaactctgtttataatttagtacagacaacccagtttaacctgggatgggcatctgttaaagtgctggaaaaaacagggaaatatttaggaaaacactggtacattatttaaaggctttntccaaggtcaggantgtttaaacttcgtttcacatttttatccntttggccacggcctgtggggcnaggatggattttttttccggccaagggtgttaaacg

SEQ ID NO: 14 - DNA, 392 bp, Homo sapiens
  misc_feature (331): n= a, c, g, or t
tgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgnaatatgttatttacagaggagaacaggtaatgtagttttaattttgtttcatcttccaaaa

SEQ ID NO: 15 - DNA, 317 bp, Homo sapiens
  misc_feature (120): n=a, c, g, or t
ttttttttgcagttctaaaacagatttatattacaaagcaatgtaaataaatactgggctagtacaaaagctcttatttacagttttacaaatgaaattgtattcagtgtaaatgctgtnttttaaagaacacagtattgtattagtaaaattagttctgttgagggcattacagtttgttaggaatcaatgcataacatataaaaggttcaagttaactctgtttataatttaggtacagacaacccagtttaaccggggaatgggcatctgttaaagtgctgaaaaaacnggganatatttaggaaaacnctgta

SEQ ID NO: 16 - DNA, 485 bp, Homo sapiens
  misc_feature (478): n=a, c, g, or t
tgcagttctaaaacagatttatattacaaagcaatgtaaataaatactgggctagtacaaaagctcttatttacagttttacaaatgaaattgtattcagtgtaaatgctgtgttttaaagaacacagtattgtattagtaaaattagttctgttgagggcattacagtttgttagaatcaatgcataacatataaaaggttcaagttaactctgtttataatttagtacagacaacccagtttaacctggaatggcatctgttaaagtgctgaaaaaacaggaaatatttacgaaaacactgtacattattaaagctttatcaagtcagaatgttaaacttcgttcacatttttatccttttgccacaggcctgtggggcaagatgattttttttcagcaaaggagtaaaacgtagagggccactggctgctttgatgattttagggttcagtgggaattagtttcctaaaataacnatttatc

SEQ ID NO: 17 - DNA, 291 bp, Homo sapiens
  misc_feature (3): n=a, c, g, or t
ttncctgcagttcagtgcaatgaanatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcct

SEQ ID NO: 18 - DNA, 371 bp, Homo sapiens
  misc_feature (27): n=a, c, g, or t
cgtagaggccactgctgctttgatganttttanggttcangtggaattngtttcttaaaataactatttatccatcgaccataccttcaggcctcataatctggtaagtaattaaatactcaggataagcctgttctcctctgtaaataacatattcagctaatgctaggccatttacactgggcctaccagtgactgaagtgatgcctggggggagaatgtgccattttcattgcactgaactgcaggnaagactttcccaagggttacccgggcaaaagagcagctgcctgtgggnaatgttacaaggtcttgtcttttgtngacctngggcaccccgtaccctcctccaattccatatacatatttga

SEQ ID NO: 19 - DNA, 341 bp, Homo sapiens
  misc_feature (300): n= a, c, g, or t
gaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctggaaaactcttccaaaaggcaatcaatatgtatatgggaattgggagggagggtactggggtgtccagtttcacaaaggacagatcttgtttacatttggccacaggcaggctggctctttttgcccgggtnaccttgggggaagtcttttcctggcagtttcagttgccatg

SEQ ID NO: 20 - DNA, 385 bp, Homo sapiens
  misc_feature (103): n=a, c, g, or t
tactaaattataaacagagttaacttgaaccttttatatgttatgcattgattctaacaaactgtaatgccctcaacagaactaattttactaatacaataangtgttctttaaaacacagcatttacactgaatacaatttcatttgtaaaactgtaaataagagcttttgtactagcccagtatttatttacattgctttgtaatataaatctgttttaggaactgcaggcggtttacaaaattttttcatatgtattgttcatttatacttcatcttacatcgtcatggattgaggtgatctttacatttggattccngggggctatggttcaggttgttaggttgggggaaagggttggggtttatccgggntttantttg

SEQ ID NO: 21 - DNA, 335 bp, Homo sapiens
  misc_feature (286): n= a, c, g, or t
gaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccgggttaaactggggttgtctgtactaaattattaaacagngttaacttggaaccnttttatatgttatggccttggttcttaaccaana

SEQ ID NO: 22 - DNA, 388 bp, Homo sapiens
  misc_feature (51): n= a, c, g, or t
gttttactcctttgctgaaaaaaaatcatcttgcccacaggcctgtggaanaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacaggcatttacactggaatacaatttcatttgttaaaactggtaantaggagcttttgtactagcccagtatttatttacatgctttg

SEQ ID NO: 23 - DNA, 401 bp, Homo sapiens
  misc_feature (51): n= a, c, g, or t
gttttactcctttgctgaaaaaaaatcatcttgcccacaggcctgtggaanaaggataaaaatgtgaacgaagttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactanttttacttaatacaatactgtgttctttnaaaacacaggcatttacactggaatacaattttcattttgttaaaactggttaaattaaggnggctttttgtactnggccccgtnttttattttacattgctttgg

SEQ ID NO: 24 - DNA, 354 bp, Homo sapiens
  misc_feature (325): n=a, c, g, or t
taattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgccgatgggagcctttatctgtcattagaaatctttctnatttaagaacttatgaatatgctgaagat

SEQ ID NO: 25 - DNA, 18 bp, Artificial Sequence
  Description of Artificial Sequence primer
tgtaaaacgacggccagt

SEQ ID NO: 26 - DNA, 19 bp, Artificial Sequence
  Description of Artificial Sequence primer
ggaaacagctatgaccatg

SEQ ID NO: 27 - DNA, 18 bp, Artificial Sequence
  Description of Artificial Sequence primer
tttgccgggtaaccttgg

SEQ ID NO: 28 - DNA, 18 bp, Artificial Sequence
  Description of Artificial Sequence primer
ccaaggttacccggcaaa

SEQ ID NO: 29 - DNA, 18 bp, Artificial Sequence
  Description of Artificial Sequence primer
gtaggcccagtgtaaatg

SEQ ID NO: 30 - DNA, 18 bp, Artificial Sequence
  Description of Artificial Sequence primer
catttacactgggcctac

SEQ ID NO: 31 - DNA, 20 bp, Artificial Sequence
  Description of Artificial Sequence primer
gagtaagttgcagggcatgt

SEQ ID NO: 32 - DNA, 20 bp, Artificial Sequence
  Description of Artificial Sequence primer
acatgccctgcaacttactc

SEQ ID NO: 33 - DNA, 20 bp, Artificial Sequence
  Description of Artificial Sequence primer
gaatcaccgcagttactaaa

SEQ ID NO: 34 - DNA, 20 bp, Artificial Sequence
  Description of Artificial Sequence primer
tttagtaactgcggtgattc

SEQ ID NO: 35 - DNA, 20 bp, Artificial Sequence
  Description of Artificial Sequence primer
ggcctgaaggtatggtcgat

SEQ ID NO: 36 - DNA, 20 bp, Artificial Sequence
  Description of Artificial Sequence primer
atcgaccataccttcaggcc

SEQ ID NO: 37 - DNA, 20 bp, Artificial Sequence
  Description of Artificial Sequence primer
tgagggcattacagtttgtt

SEQ ID NO: 38 - DNA, 21 bp, Artificial Sequence
  Description of Artificial Sequence primer
taatacgaactcactataggg

SEQ ID NO: 39 - DNA, 18 bp, Artificial Sequence
  Description of Artificial Sequence primer
atacactcaccggagaaa

SEQ ID NO: 40 - DNA, 18 bp, Artificial Sequence
  Description of Artificial Sequence primer
tttctccggtgagtgtat

SEQ ID NO: 41 - DNA, 1691 bp, Artificial Sequence
  Description of Artificial Sequence Sequence not specified as protein-coding is vector sequence
atgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggtaatgtagttttatttgttcatcttcaaaaatgctagggaggcatactttaactttttattaatctcttgaattgacaagacatattgccttaactggattttttaaaaattttatttggagataatttcagatttggaaagttacaaaaatagtaaagagaattttcttataacctttacctagatttcctaaatgttaatattttgttctcttttttactcttaccattctctccttctttccttgtgtgtgtacctatttttttgtgaactgtttgagagtaagttgcagggcatgtccctttaccattaactatttcaattgtaaatttcctaaaaacaagaagattttattcaaatttcgccagtcgttccggatttttcttagctcttataaataattgaaatcttgtatttaacagcctgtccatagcaaagaagtatataactgtgttttgctctcagtgagagccaaaagtagttctagagcagtgttgtgaactgggagtaggtatcggaatcaccgcagttactaaaatcagacatgattttagtcttatctgatacttatgaacttagtattcatcttagacttgctgattgaaaatctgaagaactgtactcagggtaaagatgttttgagaaaatgtccctagatgattctgatctacaacagtaatttagaacctcctccctaagattaggaatacttccggaaagtctgtttatctttcaagaaaatttttgtaccattatttgaatttatctttctcttccaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttagtaatacaatactgtgttctttaaaacacagcatttacactgaatacaatttcatttgtaaaactgtaaataagagcttttgtactagcccagtatttatttacattgctttgtaatataatcctgttttagaagtgcaaaaaaaaaaaaaaaaa

SEQ ID NO: 42 - protein, 119 aa, Artificial Sequence
  Description of Artificial Sequence Sequence not specified as protein-coding is vector sequence
MLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQ

SEQ ID NO: 43 - DNA, 1692 bp, Artificial Sequence
  Description of Artificial Sequence Sequence not specified as protein-coding is vector sequence
atgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggtaatgtagttttatttgttcatcttcaaaaatgctagggaggcatactttaactttttattaatctcttgaattgacaagacatattgccttaactggattttttaaaaattttatttggagataatttcagatttggaaagttacaaaaatagtaaagagaattttcttataacctttacctagatttcctaaatgttaatattttgttctcttttttactcttaccattctctccttctttccttgtgtgtgtacctatttttttgtgaactgtttgagagtaagttgcagggcatgtccctttaccattaactatttcaattgtaaatttcctaaaaacaagaagattttattcaaatttcgccagtcgttccggatttttcttagctcttataaataattgaaatcttgtatttaacagcctgtccatagcaaagaagtatataactgtgttttgctctcagtgagagccaaaagtagttctagagcagtgttgtgaactgggagtaggtatcggaatcaccgcagttactaaaatcagacatgattttagtcttatctgatacttatgaacttagtattcatcttagacttgctgattgaaaatctgaagaactgtactcagggtaaagatgttttgagaaaatgtccctagatgattctgatctacaacagtaatttagaacctcctccctaagattaggaatacttccggaaagtctgtttatctttcaagaaaatttttgtaccattatttgaatttatctttctcttccaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcaaaaaaaaaaaaaaaaaa

SEQ ID NO: 44 - protein, 119 aa, Artificial Sequence; identical to SEQ ID NO: 42
  Description of Artificial Sequence Sequence not specified as protein-coding is vector sequence

SEQ ID NO: 45 - DNA, 582 bp, Artificial Sequence
  Description of Artificial Sequence Sequence not specified as protein-coding is vector sequence
gaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaaaaaaaaaaaaa

SEQ ID NO: 46 - protein, 160 aa, Artificial Sequence
  Description of Artificial Sequence Sequence not specified as protein-coding is vector sequence
ERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 47 - DNA, 23 bp, Artificial Sequence
  Description of Artificial Sequence primer
ctccggacaacaaggtcttaacc

SEQ ID NO: 48 - DNA, 19 bp, Artificial Sequence
  Description of Artificial Sequence primer
ccacctatgtacgcatgcc

SEQ ID NO: 49 - DNA, 356 bp, Homo sapiens
tccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtgg

SEQ ID NO: 50 - DNA, 21 bp, Artificial Sequence
  Description of Artificial Sequence primer
atttaaccctcactaaaaggg

SEQ ID NO: 51 - DNA, 20 bp, Artificial Sequence
  Description of Artificial Sequence primer
aaaggctcccatcggcaaat

SEQ ID NO: 52 - DNA, 20 bp, Artificial Sequence
  Description of Artificial Sequence primer
gttgagggcattacagtttg

SEQ ID NO: 53 - DNA, 20 bp, Artificial Sequence
  Description of Artificial Sequence primer
aaaacgtagaggccactgct

SEQ ID NO: 54 - DNA, 20 bp, Artificial Sequence
  Description of Artificial Sequence primer
tggtgtagactgacgccctt

SEQ ID NO: 55 - DNA, 20 bp, Artificial Sequence
  Description of Artificial Sequence primer
tccggtgagtgtatctttcc

SEQ ID NO: 56 - DNA, 20 bp, Artificial Sequence
  Description of Artificial Sequence primer
ctcctttgtcttgggcattc

SEQ ID NO: 57 - DNA, 20 bp, Artificial Sequence
  Description of Artificial Sequence primer
atctgctctgccctcttgtt

SEQ ID NO: 58 - DNA, 20 bp, Artificial Sequence
  Description of Artificial Sequence primer
gggtatcgcggcaatttaca

SEQ ID NO: 59 - DNA, 20 bp, Artificial Sequence
  Description of Artificial Sequence primer
aacaagagggcagagcagat

SEQ ID NO: 60 - DNA, 20 bp, Artificial Sequence
  Description of Artificial Sequence primer
tgccccatctcaactaatac

SEQ ID NO: 61 - DNA, 20 bp, Artificial Sequence
  Description of Artificial Sequence primer
gtaatgccctcaacagaact

SEQ ID NO: 62 - DNA, 20 bp, Artificial Sequence
  Description of Artificial Sequence primer
ggcgtcagtctacaccactt

SEQ ID NO: 63 - DNA, 20 bp, Artificial Sequence
  Description of Artificial Sequence primer
taaattgcccgcgataccca

SEQ ID NO: 64 - DNA, 20 bp, Artificial Sequence
  Description of Artificial Sequence primer
cactcagtcactggtaggcc

SEQ ID NO: 65 - DNA, 20 bp, Artificial Sequence
  Description of Artificial Sequence primer
atctgctctgccctcttgtt

SEQ ID NO: 66 - DNA, 20 bp, Artificial Sequence
  Description of Artificial Sequence primer
tagttgagatggggcacaag

SEQ ID NO: 67 - DNA, 20 bp, Artificial Sequence
  Description of Artificial Sequence primer
aaacgtagaggccactgctg

SEQ ID NO: 68 - DNA, 20 bp, Artificial Sequence
  Description of Artificial Sequence primer
cgggtaaccttgggaaagtc

SEQ ID NO: 69 - DNA, 20 bp, Artificial Sequence
  Description of Artificial Sequence primer
gggctttactgctttacaga

SEQ ID NO: 70 - DNA, 20 bp, Artificial Sequence
  Description of Artificial Sequence primer
gtaagggctgctgacagtga

SEQ ID NO: 71 - DNA, 20 bp, Artificial Sequence
  Description of Artificial Sequence primer
ttactccagcagagggcact

SEQ ID NO: 72 - DNA, 20 bp, Artificial Sequence
  Description of Artificial Sequence primer
ctgacgcccttcaatgtctc

SEQ ID NO: 73 - DNA, 20 bp, Artificial Sequence
  Description of Artificial Sequence primer
ggtactaaggccacaattca

SEQ ID NO: 74 - DNA, 20 bp, Artificial Sequence
  Description of Artificial Sequence primer
gggtatcgcggcaatttaca

SEQ ID NO: 75 - DNA, 20 bp, Artificial Sequence
  Description of Artificial Sequence primer
gttgagggcattacagtttg

SEQ ID NO: 76 - DNA, 20 bp, Artificial Sequence
  Description of Artificial Sequence primer
taacaagagggcagagcaga

SEQ ID NO: 77 - DNA, 20 bp, Artificial Sequence
  Description of Artificial Sequence primer
agttctgttgagggcattac

SEQ ID NO: 78 - DNA, 20 bp, Artificial Sequence
  Description of Artificial Sequence primer
ggcctaccagtgactgagtg

SEQ ID NO: 79 - DNA, 20 bp, Artificial Sequence
  Description of Artificial Sequence primer
gggctagaggacctgaagag

SEQ ID NO: 80 - DNA, 20 bp, Artificial Sequence
  Description of Artificial Sequence primer
agtgccctctgctggagtaa

SEQ ID NO: 81 - DNA, 20 bp, Artificial Sequence
  Description of Artificial Sequence primer
ggcgtcagtctacaccactt

SEQ ID NO: 82 - DNA, 20 bp, Artificial Sequence
  Description of Artificial Sequence primer
tgaattgtggccttagtacc

SEQ ID NO: 83 - DNA, 20 bp, Artificial Sequence
  Description of Artificial Sequence primer
atgcccaagacaaaggagga

SEQ ID NO: 84 - DNA, 20 bp, Artificial Sequence
  Description of Artificial Sequence primer
gtaatgccctcaacagaact

SEQ ID NO: 85 - DNA, 20 bp, Artificial Sequence
  Description of Artificial Sequence primer
atctgctctgccctcttgtt

SEQ ID NO: 86 - DNA, 20 bp, Artificial Sequence
  Description of Artificial Sequence primer
cgggtaaccttgggaaagtc

SEQ ID NO: 87 - DNA, 20 bp, Artificial Sequence
  Description of Artificial Sequence primer
ccggacaacaaggtcttaac

SEQ ID NO: 88 - DNA, 3353 bp, Homo sapiens
  CDS (1)..(2352)
tgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgccgatgggagcctttatctgtcattagaaatctttctcatttaagaacttatgaatatgctgaagatttaatttgtgatacctttgtatgtatgagacacattccaaagagctctaactatgataggtcctgattactaaagaagcttctttactggcctcaatttctagctttcatgttggaaaattttctgcagtccttctgtgaaaattagagcaaagtgctcctgttttttagagaaactaaatcttgctgttgaacaattattgtgttcttttcatggaacataagtaggatgttacatttccagggtgggaagggtaatcctaaatcatttcccaatctattctaattaccttaaatctaaaggggaaaaaaaaaatc

SEQ ID NO: 89 - protein, 784 aa, Homo sapiens
CELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 90 - DNA, 3799 bp, Homo sapiens
  CDS (3)..(2270)
aagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgccattaaaccttatggggttttctgttgcagactgttgattgaccttactaaatcccgaaatctaaaaaatgaattgtggccttagtaccacaccatctttaaagtctagtgtttagtccccttttccttcaaaactttccaacaaatctagcgctttactgaactcagaacattgttctctttgagaatgtgaagattttaaatagccaaagaattttcatgtataagagctagctaaatatagtatatcctgctctttcgaagaagatacaaaactgttgcctgtactaatgggtatagtagagcagttgaagaactaacacatacatggacttttcggtctgaatttgtgttggcatccatggtacttactgttcagtaggatgttattgcaaggagcagagtgccctctgctggagtaatcgcaattattcttgcagcagattaatttgacttgggtcatgaattcaacaaccagttacttgcctttcatcatacaatttcttcggtagttgagaatttggtctacatttatcaaatgaggaaagagtgtcacaaactctaaaaagctgaaggagaccccacacatcttctcactgtcagcagcccttacttctgcaaaatgttgaaggataatgtttctctgtttgcaaagaagatgcctctggctagaatgtttgtgcagttataagcaagggactgcttgtttttgtaagttatctcaactttattcttgtgaaattgcaaaggaagatcaataaaaagacttcatttgaatgtaaatggtgtgaaatactgatgtgttttgtacatgtacataatatatttacttcctgctttcacattagtaatctgagatggttctaccattttataattagaaggagatgtaggggtgggagtggggaggg

SEQ ID NO: 91 - protein, 756 aa, Homo sapiens
AHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 92 - DNA, 2971 bp, Homo sapiens
tgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgcc

SEQ ID NO: 93 - DNA, 20 bp, Artificial Sequence
  Description of Artificial Sequence primer
gggcggaaagacgtagttga

SEQ ID NO: 94 - DNA, 20 bp, Artificial Sequence
  Description of Artificial Sequence primer
gcggctgttcaccttctcag

SEQ ID NO: 95 - DNA, 20 bp, Artificial Sequence
  Description of Artificial Sequence primer
acgcaagtgatggcagaaag

SEQ ID NO: 96 - DNA, 20 bp, Artificial Sequence
  Description of Artificial Sequence primer
tcacttgcgtggcagttgac

SEQ ID NO: 97 - DNA, 20 bp, Artificial Sequence
  Description of Artificial Sequence primer
gcggcaggtttgtagatgac

SEQ ID NO: 98 - DNA, 1568 bp, Homo sapiens
  CDS (2)..(1567)
ggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggagctgatgtccatgctaaagataaaggtgatctggtaccattacacaatgcctgttcttatggtcattatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgca

SEQ ID NO: 99 - protein, 522 aa, Homo sapiens
ARIMSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGADVHAKDKGDLVPLHNACSYGHYEVTELLVKHGACVNAMDLWQFTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNC

SEQ ID NO: 100 - DNA, 4127 bp, Homo sapiens
  CDS (2)..(3508)
  3′UTR (3509)..(4127)
ggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggagctgatgtccatgctaaagataaaggtgatctggtaccattacacaatgcctgttcttatggtcattatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgcc

SEQ ID NO: 101 - protein, 1169 aa, Homo sapiens; identical to SEQ ID NO: 2

SEQ ID NO: 102 - DNA, 32 bp, Artificial Sequence
  Description of Artificial Sequence primer
gagcattggggtctgcaccatgtcgcaaaagg

SEQ ID NO: 103 - DNA, 27 bp, Artificial Sequence
  Description of Artificial Sequence primer
ccatcctaatacgactcactatagggc

SEQ ID NO: 104 - DNA, 647 bp, Homo sapiens
  CDS (2)..(646)
ggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctc

SEQ ID NO: 105 - protein, 215 aa, Homo sapiens
ELAGGALPASAAASFQDPDGGFALPPPPRGSRGAGSPARGARGRGHGTAPDPVTAGSQAARALSASSPGGLALLLAGPGLLLRLLALLLAVAAARIMSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNA

SEQ ID NO: 106 - DNA, 4406 bp, Homo sapiens
  CDS (2)..(3787)
ggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggagctgatgtccatgctaaagataaaggtgatctggtaccattacacaatgcctgttcttatggtcattatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgcc

SEQ ID NO: 107 - protein, 1262 aa, Homo sapiens
ELAGGALPASAAASFQDPDGGFALPPPPRGSRGAGSPARGARGRGHGTAPDPVTAGSQAARALSASSPGGLALLLAGPGLLLRLLALLLAVAAARIMSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGADVHAKDKGDLVPLHNACSYGHYEVTELLVKHGACVNAMDLWQFTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 108 - DNA, 436 bp, Homo sapiens; identical to SEQ ID NO: 13
  misc_feature (334): n= a, c, g, or t

SEQ ID NO: 109 - DNA, 21 bp, Artificial Sequence
  Description of Artificial Sequence primer
cgcctgagaaggtgaacagcc

SEQ ID NO: 110 - DNA, 20 bp, Artificial Sequence
  Description of Artificial Sequence primer
acgcctcgaacagctctcgg

SEQ ID NO: 111 - DNA, 23 bp, Artificial Sequence
  Description of Artificial Sequence primer
gcgtgggcgcggccatgggactg

SEQ ID NO: 112 - DNA, 20 bp, Artificial Sequence
  Description of Artificial Sequence primer
cagcgcgaatccgccgtccg

SEQ ID NO: 113 - DNA, 620 bp, Homo sapiens
  CDS (3)..(620)
ttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcaggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgc

SEQ ID NO: 114 - protein, 206 aa, Homo sapiens
KTTTTKNTICRIVRLQQNPPQRWRWDEAPSPAAEASRLTFPTNPSRRLASRNLPSRCPATAHARDDVTCAPGAGRSWQELAGGALPASAAASFQDPDGGFALPPPPRGSRGAGSPARGARGRGHGTAPDPVTAGSQAARALSASSPGGLALLLAGPGLLLRLLALLLAVAAARIMSGRRCAGGGAACASAAAEAVEPAARELFEAC

SEQ ID NO: 115 - DNA, 1039 bp, Homo sapiens
  CDS (287)..(1039)
gtacaatattgatttacaaaaagttcctctaatcaatcctgagctaataacttactgtggaaagagtaattgatcagagccatccctccaattggagtcaactttcatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcaggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgc

SEQ ID NO: 116 - protein, 251 aa, Homo sapiens
MWKLKTRTISTVRLPKECARSEQNFQGLFFSVMVVRKFSLESPGLKTTTTKNTICRIVRLQQNPPQRWRWDEAPSPAAEASRLTFPTNPSRRLASRNLPSRCPATAHARDDVTCAPGAGRSWQELAGGALPASAAASFQDPDGGFALPPPPRGSRGAGSPARGARGRGHGTAPDPVTAGSQAARALSASSPGGLALLLAGPGLLLRLLALLLAVAAARIMSGRRCAGGGAACASAAAEAVEPAARELFEAC

SEQ ID NO: 117 - DNA, 473 bp, Homo sapiens
  CDS (3)..(473)
ctagccgaaacctgcccagccggtgcccggccactgcgcacgcgcgggacgacgtcacgtgcgctcccggggctggacggagctggcaggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgc

SEQ ID NO: 118 - protein, 157 aa, Homo sapiens
SRNLPSRCPATAHARDDVTCAPGAGRSWQELAGGALPASAAASFQDPDGGFALPPPPRGSRGAGSPARGARGRGHGTAPDPVTAGSQAARALSASSPGGLALLLAGPGLLLRLLALLLAVAAARIMSGRRCAGGGAACASAAAEAVEPAARELFEAC

SEQ ID NO: 119 - DNA, 22 bp, Artificial Sequence
  Description of Artificial Sequence primer
gttcctctaatcaatcctgagc

SEQ ID NO: 120 - DNA, 26 bp, Artificial Sequence
  Description of Artificial Sequence primer
ggaaagagtaattgatcagagccatc

SEQ ID NO: 121 - DNA, 27 bp, Artificial Sequence
  Description of Artificial Sequence primer
cgccgaagcctctcgcctcacatttcc

SEQ ID NO: 122 - DNA, 27 bp, Artificial Sequence
  Description of Artificial Sequence primer
ggaaatgtgaggcgagaggcttcggcg

SEQ ID NO: 123 - DNA, 659 bp, Homo sapiens
ggaaagagtaattgatcagagccatccctccaattggagtcaacttccatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcaggaggggccttgccagcttccgccgccgcgtcgtttcaggacccggacggcggattcgcgctg

SEQ ID NO: 124 - DNA, 669 bp, Homo sapiens
ggaaagagtaattgatcagagccatccctccaattggagtcaactttcatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcaggaggggccaggaggggccttgccagcttccgccgccgcgtcgtttcaggacccggacggcggattcgcgctg

SEQ ID NO: 125 - DNA, 659 bp, Homo sapiens; identical to SEQ ID NO: 123

SEQ ID NO: 126 - DNA, 659 bp, Homo sapiens; identical to SEQ ID NO: 123

SEQ ID NO: 127 - DNA, 659 bp, Homo sapiens; identical to SEQ ID NO: 123

SEQ ID NO: 128 - DNA, 669 bp, Homo sapiens
ggaaagagtaattgatcagagccatccctccaattggagtcaactttcatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcatgcgcgggacgacgtcacgtgcgctcccggggctggacggagctggcaggagctggcaggaggggccttgccagcttccgccgccgcgtcgtttcaggacccggacggcggattcgcgctg

SEQ ID NO: 129 - DNA, 597 bp, Homo sapiens
ggaaagagtaattgatcagagccatccctccaattggagtcaactttcatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcag

SEQ ID NO: 130 - DNA, 10 bp, Homo sapiens
gagctggcag

SEQ ID NO: 131 - DNA, 30 bp, Homo sapiens
gggctggacggagctggcaggaggggcctt

SEQ ID NO: 132 - DNA, 5002 bp, Homo sapiens
  CDS (229)..(4383)
ggaaagagtaattgatcagagccatccctccaattggagtcaactttcatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcaggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggagctgatgtccatgctaaagataaaggtgatctggtaccattacacaatgcctgttcttatggtcattatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgcc

SEQ ID NO: 133 - protein, 1385 aa, Homo sapiens
MWKLKTRTISTVRLPKECARSEQNFQGLFFSVMVVRKFSLESPGLKTTTTKNTICRIVRLQQNPPQRWRWDEAPSPAAEASRLTFPTNPSRRLASRNLPSRCPATAHARDDVTCAPGAGRSWQELAGGALPASAAASFQDPDGGFALPPPPRGSRGAGSPARGARGRGHGTAPDPVTAGSQAARALSASSPGGLALLLAGPGLLLRLLALLLAVAAARIMSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGADVHAKDKGDLVPLHNACSYGHYEVTELLVKHGACVNAMDLWQFTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 134 - DNA, 4992 bp, Homo sapiens
  CDS (876)..(4373)
ggaaagagtaattgatcagagccatccctccaattggagtcaacttccatgactgttcggatttcctttattttgggggcagttcatccaaacttctattaaacggcaactagttcacttttgagaagtggtttacaagaaacaacaacaacaacaacaaagcagttgcggaggaaagaaaagagacaaagtaaaaaaaacggaaaagaaatctcccaggagaaagggatgtggaagctgaaaacacggacaatttccacagtaagacttccaaaagaatgtgcaagatccgagcaaaactttcaagggctctttttcagtgtaatggtagtgagaaagttcagcctggaaagcccagggcttaaaacaacaacaacaaaaaacacaatatgcaggatcgttcggcttcagcagaacccaccgcaaagatggcggtgggacgaagccccttctcccgccgccgaagcctctcgcctcacatttcccacaaacccttcgcgccgcctcgctagccgaaacctgcccagccggtgcccggccactgcgcacgcgcgggacgacgtcacgtgcgctcccggggctggacggagctggcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggcccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggcctgcgcgagcgccgcggccgaggccgtggagccggccgcccgagagctgttcgaggcgtgccgcaacggggacgtggaacgagtcaagaggctggtgacgcctgagaaggtgaacagccgcgacacggcgggcaggaaatccaccccgctgcacttcgccgcaggttttgggcggaaagacgtagttgaatatttgcttcagaatggtgcaaatgtccaagcacgtgatgatgggggccttattcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggagctgatgtccatgctaaagataaaggtgatctggtaccattacacaatgcctgttcttatggtcattatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatgtgtcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcaacagaactaattttactaatacaatactgtgttctttaaaacacagcatttacactgaatacaatttcatttgtaaaactgtaaataagagcttttgtactagcccagtatttatttacattgctttgtaatataaatctgttttagaactgcagcggtttacaaaattttttcatatgtattgttcatctatacttcatcttacatcgtcatgattgagtgatctttacatttgattccagaggctatgttcagttgttagttgggaaagattgagttatcagatttaatttgcc

SEQ ID NO: 135 - protein, 1166 aa, Homo sapiens
MSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGADVHAKDKGDLVPLHNACSYGHYEVTELLVKHGACVNAMDLWQFTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 136 - DNA, 3045 bp, Homo sapiens
  CDS (1)..(3042)
atggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcagttcatgaaattatatgaagaaaaaaccgggaacgcttggcactccaaaaatttcacgaagtatcccaaaaagttttaccccctggagattgactatggccaggatgaagaggcagtgaagaagctcacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacagtgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggtgtgatagacacctctctactatataacgagtacattgtctatgatattgctcaggtaaatctgaagtatctgctgaaactgaaattcaattttaagacctccctgtggtaa

SEQ ID NO: 137 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEQFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHSAYDLEVIDIFKIEREGECQRYKPFKQLHNRRLLWHGSRTTNFAGILSQGLRIAPPEAPVTGYMFGKGIYFADMVSKSANYYHTSQGDPIGLILLGEVALGNMYELKHASHISRLPKGKHSVKGLGKTTPDPSANISLDGVDVPLGTGISSGVIDTSLLYNEYIVYDIAQVNLKYLLKLKFNFKTSLW

SEQ ID NO: 138 - DNA, 5482 bp, Drosophila melanogaster
  CDS (474)..(4016)
aaacaatgcaatatttcgcggagcagtgaattaatccggaaataatcgtccgtgcccagagctttggaggccaagtaccagggagctagtcccagagttggtcgcagtctcagtaaaaacgaatcgtagccaaccgcgatccttgcaaccgtgctgtgtcgaaccaaagaaatcctattgattttgggtctgcaattgtgcattaaatattaagcaaaaacgagggctggtcgcgtggcagccagtggcaaattgttgctcctgcggcataggcaggacacctggatacaggatgcgggcaagccagcgacggacaacggcgaggcttgtgtaggacgggcagagcaactgctggaggagagaactggactgggagtggaaaacccgaaagcccactgaatattgcgcttgttttttgttgcctatttttttcggggcgtgtgtgtgccaaagcgtagcaaacaagcacaacaatggccaacagcagccgaagtcgggccattttgagcgttaatctcgatgcggtcatggccaacgatccgctgagggagctctccgaggcctgcaaaacgggcgagatcgccaaggtgaagaagctaataacgcctcagaccgtgaacgccagggatacggcgggacgcaaatccacaccattgcatttcgcagcgggttatggacgccgggaagtggttgaattcctgctgaacagcggcgcctccatacaggcgtgtgacgagggtgggctgcacccgctgcacaactgttgctcctttggccacgccgaggtagttcgattgttgctgaaggcaggtgccagtccaaacaccaccgacaactggaactacacgccattgcacgaggcggccagcaagggcaaggtggatgtgtgcctggctctgttgcagcatggcgcaaaccatacgatccgcaactcggagcagaagacaccactggagctggcggacgaggcgacgcgtcccgtattgaccggcgaatatcgaaaggatgagctgcttgaagccgcacgctcgggggccgaggatcgcctgctggccctactcacgccactcaatgtcaactgtcatgccagcgatggacgacgctcaacgccgctccatctggcagcgggctacaatcggatcggcatcgtggaaattctgctggccaacggagcggatgtacatgctaaggacaagggcggtctggtgccgctgcacaatgcctgctcctacggacacttcgatgtgaccaagctgcttatccaggcgggcgccaatgtcaacgccaacgatctgtgggcctttacgccgctccacgaggccgcctccaaaagtcgcgtcgaggtctgcagcctgctgctcagtcgtggagcggatcccaccctcctaaactgccacagcaagtcggccatcgatgcggcgcccaccagggagctgagagagcggattgcctttgaatacaagggtcactgcctgctggacgcctgtcgaaagtgtgatgtgtcccgtgccaagaagctggtatgcgcagagattgttaacttcgtgcatccatatacaggagacactccgctccacctggccgttgtcagtccggatgggaagcgcaagcagctgatggaactgctgaccagaaagggatccttgctgaacgagaaaaacaaggctttcctcacgcccctgcatttggctgccgagctgcttcactacgatgccatggaggtgctgctaaagcagggcgccaaggttaatgcattggacagtcttggacaaacgccactgcatcggtgcgcccgtgatgagcaagcggtgcgactgctgctctcgtacgcagcggacacgaatatcgtttcccttgagggacttacggccgctcaattggcctcggacagcgtgctgaagctgctcaagaatcctccggacagtgagacacatttactggaggcagccaaggcgggagatctggacactgtgcgccgtatagtgctcaacaatccgatttcggtcaattgccgggatttggacggacgacattccacacctttgcactttgctgctgggtttaatagagtgccagtggttcagtttcttttggaacacggcgccgaggtttatgcggctgacaagggcggactggtgcccctgcacaatgcctgctcttatgggcactatgaggtaaccgaactgctggtcaagcacggagccaatgtaaatgtatcggatttgtggaagtttactcctcttcatgaagctgccgccaagggaaagtatgatatttgcaagctgctcttgaaacatggcgctgatccaatgaagaagaatcgggatggcgcgacaccagcggatttggttaaggaatctgatcacgatgttgcagagctgctgagaggaccgtccgctctgctagacgcagcaaagaaaggaaacttggcacgggtacagcgattggttacaccggaatccattaattgccgggacgcgcagggcaggaattccacaccacttcacctggccgccggatataacaactttgagtgtgccgagtaccttctggagaatggagccgatgttaatgcacaggacaaggggggactaatacctctgcacaatgccagcagctatgggcatttggatattgcggcactgctaattaagcacaagacggttgtcaatgcgacagataaatggggattcacaccgctccacgaggctgcacagaaggggcgcactcaattgtgctcgctcttgttggcccacggtgccgatgcctatatgaaaaaccaggaggggcagacgcccattgagttggccacggcagatgatgttaagtgcttgctccaggacgcgatggccacctcgttgagtcaacaggcgttgagtgcttccacgcaatcgctgacaagcagttccccggcaccagatgcaactgctgctgcggctccgggcacatcttcatcgtcctcatccgcaatcctatcgcccaccacggaaacggtgttgctgcccaccggtgcctccatgattctgagtgttcctgttccacttccactgtccagtagcacgcgcatcagtcccgcccaaggagcagaggccaatggggctgagggctcctcttcggatgatctactgccggatgcggataccataacaaatgtgtccggattcctaagcagccagcagctgcatcatctaatcgaactgttcgagcgcgaacaaatcaccttggacattctagccgagatgggccacgacgatctcaagcaggtgggcgtctccgcctacggcttccgccacaagatactcaagggaatcgcccagctgaggtccaccacaggcattggtaacaacgtgaatctatgcacattgttggtggacttgctgccggacgataaggagtttgtggccgtcgaggaggagatgcaggccacgattcgtgaacatcgtgataatggacaggctggaggttatttcactcgatataacatcattcgggtgcaaaaggtacaaaatcgaaagctgtgggagcgttatgctcatcgacggcaagagatcgccgaggagaatttcctgcagtccaacgagcgtatgctcttccacggtagtcccttcatcaacgcaattgtgcaacgcggattcgacgagcgccacgcctacattggcggcatgtttggggctggcatttatttcgccgagcatagctcgaaaagcaaccagtatgtgtacggaattggcggcggcattggctgtccctcgcacaaggataagtcctgctacgtgtgtcctagacaattgctgctgtgccgagtggcgttaggcaaatccttcttgcaatacagtgcaatgaagatggcccatgcaccgccgggacaccactcggtggtgggcagaccctcggcgggtggcttgcatttcgccgaatacgttgtctatcggggcgaacagtcttatccggagtacttgataacctaccaaatcgtcaagcccgatgacagcagtagtggaacggaggatacaagatgatggatgccctctgtcgggtccacgcccacaaccacgtcgcccgcgctgcaccagccgcaaacgcaacaacaaccgcagcagcaacagcagcagcagccgcaaccacaacaacagcagaaggcaccactgccgttgccaccgccacaacagcagacctcagctccagttgccaagaggcggccgaaacatgccaaaccatcgctgcagttgcagtatcagccctatcagccccagcaccacccggttgttgcaaccgccgctgctgtgaccaccacccaaccttcgcccgctggcgtttttgcgcacagcaataacaacaataatacgagcagcggaaatgtgaataataacaacaatgacatgtcgccggtgtcgaacagcaatagctactcctcggtggacaccaaccagacgctgctcaactcgctggccaaccagcagcgcaaccatcgacagccacagaatcatcatcatcagcagcagcagcaggcgaatcgcagccaaaagtatagtcaatttatgatcatcacacccgccgtttccatagatcgcgacttcgagtacgagtcgcatttggactttgaggatttcgccaatgcggcccacaacaatggcaatctgtttcgacttggattgcggcggagtgatagcagcagcgacgacagcggccacagcagcgatagcagcagtttccgctcgaattacaatccctacttgcatcacagtcgccagcatttgctatcgaaaggtggtaatggtggtggcggcggtgctagtcgtcacttctacgccttcacctcgtcgtggcgctggtgcagtcttctgtgcgccgccatgcgctgctttggggccgggggagccgggcacgggaatgctccgtacagcagctcgctgcaacatcatcgactaagacgctgttcgtcgtacaatgcggagaatgcctacgagcactttgcggcccccttcaaggcgcgcaagacgcgcgaccacatgaataacatcacctacgagttgtgacggtggtacgacatgctggtggttattgatctctatgtctccgttggcctcccgtctattttattacaattactagctatagatgtcgtgtcctgtgtgtctctctctctctcgttgtttattatattactctataatatatcacgtaagggcgagctagcgagatggattcgttcggtttggatcgaattggattggattcgattgggttaatcaaaagtgaagcacagtttttgagtgattttaattcgaaatacggaaaatgcgattcgattatacgaggttacaagttctttgccgatgaatgcattacattacattacattacgctcgcgcgtttatttaagtgtttaagcttagttaatttaaacaataattaaaactccaattaaatttaaatatacaaatacatatacatcaatcgaaaaaaaaaaaaaaaaaaaaaa

SEQ ID NO: 139 - protein, 1181 aa, Drosophila melanogaster
MANSSRSRAILSVNLDAVMANDPLRELSEACKTGEIAKVKKLITPQTVNARDTAGRKSTPLHFAAGYGRREVVEFLLNSGASIQACDEGGLHPLHNCCSFGHAEVVRLLLKAGASPNTTDNWNYTPLHEAASKGKVDVCLALLQHGANHTIRNSEQKTPLELADEATRPVLTGEYRKDELLEAARSGAEDRLLALLTPLNVNCHASDGRRSTPLHLAAGYNRIGIVEILLANGADVHAKDKGGLVPLHNACSYGHFDVTKLLIQAGANVNANDLWAFTPLHEAASKSRVEVCSLLLSRGADPTLLNCHSKSAIDAAPTRELRERIAFEYKGHCLLDACRKCDVSRAKKLVCAEIVNFVHPYTGDTPLHLAVVSPDGKRKQLMELLTRKGSLLNEKNKAFLTPLHLAAELLHYDAMEVLLKQGAKVNALDSLGQTPLHRCARDEQAVRLLLSYAADTNIVSLEGLTAAQLASDSVLKLLKNPPDSETHLLEAAKAGDLDTVRRIVLNNPISVNCRDLDGRHSTPLHFAAGFNRVPVVQFLLEHGAEVYAADKGGLVPLHNACSYGHYEVTELLVKHGANVNVSDLWKFTPLHEAAAKGKYDICKLLLKHGADPMKKNRDGATPADLVKESDHDVAELLRGPSALLDAAKKGNLARVQRLVTPESINCRDAQGRNSTPLHLAAGYNNFECAEYLLENGADVNAQDKGGLIPLHNASSYGHLDIAALLIKHKTVVNATDKWGFTPLHEAAQKGRTQLCSLLLAHGADAYMKNQEGQTPIELATADDVKCLLQDAMATSLSQQALSASTQSLTSSSPAPDATAAAAPGTSSSSSSAILSPTTETVLLPTGASMILSVPVPLPLSSSTRISPAQGAEANGAEGSSSDDLLPDADTITNVSGFLSSQQLHHLIELFEREQITLDILAEMGHDDLKQVGVSAYGFRHKILKGIAQLRSTTGIGNNVNLCTLLVDLLPDDKEFVAVEEEMQATIREHRDNGQAGGYFTRYNIIRVQKVQNRKLWERYAHRRQEIAEENFLQSNERMLFHGSPFINAIVQRGFDERHAYIGGMFGAGIYFAEHSSKSNQYVYGIGGGIGCPSHKDKSCYVCPRQLLLCRVALGKSFLQYSAMKMAHAPPGHHSVVGRPSAGGLHFAEYVVYRGEQSYPEYLITYQIVKPDDSSSGTEDTR

SEQ ID NO: 140 - DNA, 20 bp, Artificial Sequence
  Description of Artificial Sequence primer
ggcctgaaggtatggtcgat

SEQ ID NO: 141 - DNA, 20 bp, Artificial Sequence
  Description of Artificial Sequence primer
tgagggcattacagtttgtt

SEQ ID NO: 142 - DNA, 346 bp, Homo sapiens
ggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctca

SEQ ID NO: 143 - DNA, 29 bp, Artificial Sequence
  Description of Artificial Sequence primer
gccgaattcggcctgaaggtatggtcgat

SEQ ID NO: 144 - DNA, 33 bp, Artificial Sequence
  Description of Artificial Sequence primer
gccgaattctagatgagggcattacagtttgtt

SEQ ID NO: 145 - DNA, 362 bp, Homo sapiens
gaattcggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacagtgttttctaaatatttcctgttttttcagcactttaacagatgccattccaggttaaactgggttgtctgtactaaattataaacagagttaacttgaaccttttatatgttatgcattgattctaacaaactgtaatgccctcatctagaattc

SEQ ID NO: 146 - DNA, 5616 bp, Homo sapiens
tgaatgcctgctggtgaaggccagatcagatttcaacctgggactggattacagaggattgtttctaataacaacatcaatattctagaagtccctgacagcctagaaataagctgtttgtcttctataaagcattgctatagtgatgaatagtatgagtaactgatacatactcaactgctactgttccctttgaggaaatgtttacaggggcggccttttaacatatctcaggctcattttcattgcaattatccatttctaaaacaagattgcttcgatctagacttggaaatggaaaataagaaaaccaatgctttttcaaatgttcacaattcacacactacatttgttttgttatgcatgacgtgtctataacaaatatacacatacgacaggcaacaagcttgtttttgatttgccagacatgcatcattggctattgtttgtttgttttttgtttttttgtgttttttgggttactttgaaaatgagccagagccttcttgaggatattttgcacaaagtcacgctgacaaaatcattagcagtgcaacccaagcttctggctgagcaagattcagtttccactttttaaaatttttttattttgctctgtagctgcacttctcgttatcataaattgagatgaaaaggaaaaaacatcaagttttagtacctttttatgaattggcctatcttacaagagaagggcacaaacaccaacctgacttaggaacgcctaaattcagagaagtcaaagccggtgaaggccacttgctctttccaacacaagcctgccacagaggtcttcgggacagtactggagatgcaggttgacacgggcttgagttccaaggtgaaaaaactggggaggctgtgaaggaagagctgcattaaggagggtgaggagcgtgtggttctgtatcatggcagccccaatggatccaggggatgcctccaaaaaatacatgcttcccttcccttaatctgtactgttgggattgttacccctccaaattagctgccttatttcaaaagtcagtgaaattactgcacttgatgagggtcacaaaaataccacttgattgtttctttagttgagaatgctgggattcagactcgaatagtggatagatacacacaaatgcaaggacttttttgtttactccagatttggggtttattttgagtggcatgcttcaaatagttcataaagatccttgcattaaatttctgaaccatttcttcaaacttcttagtgtgtttagacaaggagaacaaaaattgaaaccaaagccctttctgttattttttcaatgaaggtgagaaagaaataccatacaattttctttgtgaaattactgtttattttcatcaacatttaccaagtgccattgacatttataaaaaaaatgatcctttatagttcttacacttgcccttttcaccttaactgaatatgaattgagtgcactaacttatttacttgatatactgtgcatctactctgctttgaagcgaaagaaatataaacacgaggaggaataggaaagacagtgtgacacaaacttgccattgcaattcaaagccctgaaaacgatgggtttaatgcaaggtgattaagctgtgacctcctttaatctcctgaagcaaaataaaatggttacatgcaaaacttctagaaatacactcttaaaatatatacattttgctttgattttggcttcaacccagtgctggaactaggcatccagactagtttgaatgtttgtagctgaatttttatgggtcctcaaaattaaatcgagaattagcctcagttgttgcttcttttgaagtttcagtgacccaagctgggtgtttgtgtcttggctacttgtttaatagcactagaattccaggtgaagctttgagagttgatattcattaagagggctttttttccccttctttccttctcttttgctgtaacaaagggttgaagaaattgccatctgtgtagttttcagtagctgtcaagtgtgtcttacttaccttcccccagacgtagtttaaaatggtaaacacagctgtgatttttagttaagtaaaagagttaatatgatatagatatggaaagctttatggcttcattaaaaagataaaccactacctaactgtggttgtatgttgtttccatcatactaactagatgaatggatgcgccagttttcatcttggtccttacacttgagaagttaaactgtggttcagtatttaaactgccagtgttatacgtctcatgctctgtgtgccaggtgaaggtactgtgtaaggaagacatttgcggtgcttcttgtcctataatgattcaagtatatagtagttcttgaaagagtgtgcatatattactcatctgcttaagagagtgggttaatggatatatcagaggagccaaatacatttttttcagaacttgaaaaccaaaggtcatcatgagtgcactcaaaagttaggacaagtttattacatttgggattttcatctgtagccgtatgaagaaccctttccaatataaaagcatggcattaaattaggctgaagtcttttattttttgtatatgtactatatagaaatactagcaagttaggatcatccaatatggcctaccccgaaatggcccctctgtttccctaaccacatggaagaaagaatctgaacgtctccaccggctctacccgagttccaaaactaaagggcttctccagacctgatggttccagtttacctgctgttggcctgctggatacttgactcaggcataaattaagtgccctggtcccgaactttctcccagtatttgacctccttccctctttcctaaattactagtctggaattaaaattagctccagcaatgacctttgactccattcattttctcctcatcttgggtcttaaaaaaggagaccagatacctcctagcttttgtatcacaaccaggaatgggtattaggcctcatgcgctttgctcagaacactgccgctttgttaacaaatgacagcatggaacccagagttttgattcgatgcaaaataacagcagtgcaaccaggattcttgttttccttttccttcttggagtttggaatttctagcttttcaagcagcataagtagaatcaacattaggatgttttcatgaaatagcatccttatacttctttgagcttgatgttagtggctagactgatttccctttgctctcaaaatacaaagtgcattgaagtatacagagaaatgcctgaatatggcaagcaaataatgtagattaacattctattattgtatccgttttacaaaaaataaaattttgatatatgccggagaacggcattagaatgcaataagttgtctaggtttttctgtttcagtgtctctcccaatggcacgaagggttattgggcattgtccccacccccgcctttttaacatgtgcactatctggattcctgtaaatggccttgcaaacagaagtggtgtgtattttcaagcacctttcccccattgtatccgaatccctcttgtgtgatatctgtgacaaataccattcttcttgtgttttctgttgggactaattgtctcacgtaaagctatagaccttactaatttggcaggtattcaaaactgccattaagataggatttcatgtcagatacgtatttaaagagtaaagtcaaatttgtttaatgtcagatcagtgacagaagtgaaaagaaagtaattgtgaaagtgatgtttgagctattgtacacatctagcatatggaaagcaaatgcactcgaaaactactattctagaacatgaggcttcttcagcaacttgtgcactctgccattaataaattaaatttttcccctctagaaagccttaactatggcggaaactttttaaccttttatattttaataaataaaacattgtagtcccatttcttagtgtttgaaaggtgtgtcagtgagtcggccatgtctccatgtgtttcagacctgttcatcttattttatgatggtatatttcataagtaatattcccttacatgcaatggagctgattaaaattaatccatttcaatttctccatattggaacttcctcagctaccagatttctggtttggagaagtgctggaaagatttcaaagcctattcagttgtgtatgtggggatacgacagcaactgtgataccttgtagaatatgagtgatatgcaagctgtgttttttaattgttttaaaatgtaaattatggttatgctaaagtgaaaacctagaggaagctaatgattttatatactttgcacgaccaaatatggtcgtagtatgacgagttttatacattgccagagagttctgcctcctctgaaataacattcgcactgtagattgcatttcggcttttcctcctttcacattcttttttgctttacacttcacgtcttcgcacctgccctacctcccatcctttcaaagaggtttctttcacgttccagaattcagattgttctgtgatttcttttacatcagtctacccatttctgcaggcagccctgaaagcccttgtgttgattcagagtgtttgcagagaaatgcagttgaaccctggtagtggggtgtccctcacacacccgcgcacccctcccaaagttcaggatgaaaggctagaaaacccattcaaagttaggaaagaacacagatctttgaggccgatagcctagacctagaagatgaccttgagtatgtaaacattgtctccgtgacacaaaacactgaaactcttcatgtgcatataacacctgcttctgctcccattgtttcaagctcatcttatctttgtagtagtaatgtttgtctttgatacctacaaactaaaaaggtacttttatcaaggtttctcaaaacatttacaaaaccagctttgagaaaatgttatgttgcctggcaacagcactcggagtagtaattgtgttttctcattgtgatgttggtctgtgtgagcaaccagtgtagtgactctttggttcattattcgtgttgtttttatttttagtctctgtgtgacccaacagtggcaggggttacaaccccctctcctttcttttttgtatttatctatttgtaggattgtcagatcaagtacaagatgcccagttaagtttgaatttcagagaaacaatttcacgttaagaatgtttcatgcaatatttggcatatatttacagtaaaagcattcattatttgtctgaaattcaaatttaactgagcatgctggtttttctcattgtttggtttttctaaatctggcaatcctacagctgtggtcatgggaaatcacctacagcatgttaaagtcctctagtcatcatctcgtcacctgaaatggaagtcctttttccctcaccctccacttctttccaaaggagggcatcaaggaacttaacctgcctgcctggtgggtttctatttaagacatctttgtgattatatttaacctgcaattgtgctttggcttaatgtctagctcactgtacttgtaaatgattaatattcaataaaaccatttttaaagtaaaaaaa

SEQ ID NO: 147 - DNA, 29 bp, Artificial Sequence
  Description of Artificial Sequence primer
gccgaattccttgtttttgatttgccaga

SEQ ID NO: 148 - DNA, 34 bp, Artificial Sequence
  Description of Artificial Sequence primer
gccgaattccggctttgacttctctgaatttagg

SEQ ID NO: 149 - DNA, 372 bp, Homo sapiens
gaattccttgtttttgatttgccagacatgcatcattggctattgtttgtttgttttttgtttttttgtgttttttgggttactttgaaaatgagccagagccttcttgaggatattttgcacaaagtcacgctgacaaaatcattagcagtgcaacccaagcttctggctgagcaagattcagtttccactttttaaaatttttttattttgctctgtagctgcacttctcgttatcataaattgagatgaaaaggaaaaaacatcaagttttagtacctttttatgaattggcctatcttacaagagaagggcacaaacaccaacctgacttaggaacgcctaaattcagagaagtcaaagccggaattc

SEQ ID NO: 150 - DNA, 1320 bp, Homo sapiens
  CDS (1)..(1317)
atggcggaggatgtttcctcagcggccccgagcccgcggcggtgtgcggatggtagggatgccgaccctactgaggagcagatggcagaaacagagagaaacgacgaggagcagttcgaatgccaggaactgctcgagtgccaggtgcaggtgggggcccccgaggaggaggaggaggaggaggaggacgcgggcctggtggccgaggccgaggccgtggctgccggctggatgctcgatttcctctgcctctctctttgccgagctttccgcgacggccgctccgaggacttccgcaggacccgcaacagcgcagaggctattattcatggactatccagtctaacagcttgccagttgagaacgatatacatatgtcagtttttgacaagaattgcagcaggaaaaacccttgatgcacagtttgaaaatgatgaacgaattacacccttggaatcagccctgatgatttggggttcaattgaaaaggaacatgacaaacttcatgaagaaatacagaatttaattaaaattcaggctatagctgtttgtatggaaaatggcaactttaaagaagcagaagaagtctttgaaagaatatttggtgatccaaattctcatatgcctttcaaaagcaaattgcttatgataatctctcagaaagatacatttcattccttttttcaacacttcagctacaaccacatgatggagaaaattaagagttatgtgaattatgtgctaagtgaaaaatcatcaacctttctaatgaaggcagcggcaaaagtagtagaaagcaaaaggacaagaacaataacttctcaagataaacctagtggtaatgatgttgaaatggaaactgaagctaatttggatacaagaaaaagtgttagtgacaaacagtctgcggtaactgaatcctcagagggtacagtatccttattgaggtctcacaagaatcttttcttatctaagttgcaacatggaacccagcaacaagaccttaataagaaagaaagaagagtaggaactcctcaaagtacaaaaaagaaaaaagaaagcagaagagccactgaaagcagaatacctgtttcaaagagtcagccggtaactcctgaaaaacatcgagctagaaaaagacaggcatggctttgggaagaagacaagaatttgagatctggcgtgaggaaatatggagagggaaactggtctaaaatactgttgcattataaattcaacaaccggacaagtgtcatgttaaaagacagatggaggaccatgaagaaactaaaactgatttcctcagacagcgaagactga

SEQ ID NO: 151 - protein, 439 aa, Homo sapiens
MAEDVSSAAPSPRRCADGRDADPTEEQMAETERNDEEQFECQELLECQVQVGAPEEEEEEEEDAGLVAEAEAVAAGWMLDFLCLSLCRAFRDGRSEDFRRTRNSAEAIIHGLSSLTACQLRTIYICQFLTRIAAGKTLDAQFENDERITPLESALMIWGSIEKEHDKLHEEIQNLIKIQAIAVCMENGNFKEAEEVFERIFGDPNSHMPFKSKLLMIISQKDTFHSFFQHFSYNHMMEKIKSYVNYVLSEKSSTFLMKAAAKVVESKRTRTITSQDKPSGNDVEMETEANLDTRKSVSDKQSAVTESSEGTVSLLRSHKNLFLSKLQHGTQQQDLNKKERRVGTPQSTKKKKESRRATESRIPVSKSQPVTPEKHRARKRQAWLWEEDKNLRSGVRKYGEGNWSKILLHYKFNNRTSVMLKDRWRTMKKLKLISSDSED

SEQ ID NO: 152 - DNA, 39 bp, Artificial Sequence
  Description of Artificial Sequence primer
gccccggggatcctcatggcggaggatgtttcctcagcg

SEQ ID NO: 153 - DNA, 33 bp, Artificial Sequence
  Description of Artificial Sequence primer
tcccggggatcctcacaccaggcccgcgtcctc

SEQ ID NO: 154 - DNA, 201 bp, Homo sapiens
  CDS (1)..(201)
atggcggaggatgtttcctcagcggccccgagcccgcggggctgtgcggatggtagggatgccgaccctactgaggagcagatggcagaaacagagagaaacgacgaggagcagttcgaatgccaggaactgctcgagtgccaggtgcaggtgggggcccccgaggaggaggaggaggaggaggaggacgcgggcctggtg

SEQ ID NO: 155 - protein, 67 aa, Homo sapiens
MAEDVSSAAPSPRGCADGRDADPTEEQMAETERNDEEQFECQELLECQVQVGAPEEEEEEEEDAGLV

SEQ ID NO: 156 - DNA, 38 bp, Artificial Sequence
  Description of Artificial Sequence primer
cgcaggatccccttcactcctcttcatgaggcagcttc

SEQ ID NO: 157 - DNA, 48 bp, Artificial Sequence
  Description of Artificial Sequence primer
ggatccgctaaatatctgtatctccatctttaacaagatccaaaggag

SEQ ID NO: 158 - DNA, 21 bp, Artificial Sequence
  Description of Artificial Sequence primer
gccgacttcgagtttgagcag

SEQ ID NO: 159 - DNA, 1103 bp, Homo sapiens
  CDS (9)..(1094)
ggatccccttcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttatggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatatttagcggatc

SEQ ID NO: 160 - protein, 362 aa, Homo sapiens
FTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDI

SEQ ID NO: 161 - DNA, 39 bp, Artificial Sequence
  Description of Artificial Sequence primer
cgtcgacccatggcggagtcttcggataagctctatcga

SEQ ID NO: 162 - DNA, 39 bp, Artificial Sequence
  Description of Artificial Sequence primer
ggaaacgcgtttggtgccaggatttactgtcagcttctt

SEQ ID NO: 163 - DNA, 39 bp, Artificial Sequence
  Description of Artificial Sequence primer
cttaaacgcgttgaaggacaaacacctttagatttagtt

SEQ ID NO: 164 - DNA, 79 bp, Artificial Sequence
  Description of Artificial Sequence primer
gtcgaaagcggccgcttagcctccgaactgtggatgcctccacgctccatcgaccataccttcaggcctcataatctgg

SEQ ID NO: 165 - DNA, 17 bp, Artificial Sequence
  Description of Artificial Sequence primer
tttgttcgcccagactc

SEQ ID NO: 166 - DNA, 22 bp, Artificial Sequence
  Description of Artificial Sequence primer
tatgtttcaggttcagggggag

SEQ ID NO: 167 - DNA, 20 bp, Artificial Sequence
  Description of Artificial Sequence primer
gcggaagctggaggagtgac

SEQ ID NO: 168 - DNA, 20 bp, Artificial Sequence
  Description of Artificial Sequence primer
gtcactcctccagcttccgc

SEQ ID NO: 169 - DNA, 20 bp, Artificial Sequence
  Description of Artificial Sequence primer
aagccctgaagaagcagctc

SEQ ID NO: 170 - DNA, 20 bp, Artificial Sequence
  Description of Artificial Sequence primer
gagctgcttcttcagggctt

SEQ ID NO: 171 - DNA, 20 bp, Artificial Sequence
  Description of Artificial Sequence primer
cagacacccaaccggaagga

SEQ ID NO: 172 - DNA, 20 bp, Artificial Sequence
  Description of Artificial Sequence primer
tccttccggttgggtgtctg

SEQ ID NO: 173 - DNA, 20 bp, Artificial Sequence
  Description of Artificial Sequence primer
tccgcctccaccaagagcct

SEQ ID NO: 174 - DNA, 20 bp, Artificial Sequence
  Description of Artificial Sequence primer
aggctcttggtggaggcgga

SEQ ID NO: 175 - DNA, 20 bp, Artificial Sequence
  Description of Artificial Sequence primer
tggcctggtggacatcgtta

SEQ ID NO: 176 - DNA, 20 bp, Artificial Sequence
  Description of Artificial Sequence primer
taacgatgtccaccaggcca

SEQ ID NO: 177 - DNA, 3308 bp, Artificial Sequence
  Description of Artificial Sequence Parpla-Tank 2b Fusion
atgagaggctcccatcaccatcaccatcacgattacgatatcccaacgaccgaaaacctgtattttcagggcgccatggatccggaattcaaaggcctacgtcgacccatggcggagtcttcggataagctctatcgagtcgagtacgccaagagcgggcgcgcctcttgcaagaaatgtagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgtcaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaaacgcgttgaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggagcgtggaggcatccacagttcggaggctaagcggccgc

SEQ ID NO: 178 - protein, 1099 aa, Artificial Sequence
  Description of Artificial Sequence Parpla-Tank 2b Fusion
MRGSHHHHHHDYDIPTTENLYFQGAMDPEFKGLRRPMAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYVKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKRVEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDGAWRHPQFGG